SEQUENCE LISTING (236 sequences)

SEQ ID NO: 1 - protein, 123 aa, Homo sapiens
EVQLVESGPEVKKPGTSVKVPCKASGFTFTSSAVQWVRQARGQRLEWIGWIVVGSGNTNYAQKFQERVTITRDMSTSTAYMELSSLRSEDTAVYYCAAPSCSGGRCYDGFDIWGQGTMVTVSS

SEQ ID NO: 2 - protein, 108 aa, Homo sapiens
DIVMTQSPGTLSLSPGERATLSCRASQSVSSSYLAWYQQKPGQAPRLLIYGASSRATGIPDRFSGSGSGTDFTLTISRLEPEDFAVYYCQQYGTSPWTFGQGTKVEIK

SEQ ID NO: 3 - protein, 121 aa, Homo sapiens
EVQLVESGGGLVKPGGSLRLSCAASGFTFSDYYMSWIRQAPGKGLEWVSYIGSSGSSIHYADSVKGRFTISRDNAKNSLYLQMNSLRAEDTAVYYCARDGSYGDYVRGYWGQGALVIVSSV

SEQ ID NO: 4 - protein, 107 aa, Homo sapiens
EIVMTQSPSTLSASVGDRVTITCRASQTISSWLAWYQQKPGKAPNLLIYKASTLESGVPSRFSGSGSGTEFTLTISSLQPDDFATYYCQQYNSYPWTFGQGTKVEIK

SEQ ID NO: 5 - protein, 124 aa, Homo sapiens
EVQLVESGGGLVQPGGSLRLSCEASGFTFSSYALSWVRQAPGKGLEWVSAISGSGGSIYYADSVKGRFTISRDNSKSTLYLQMNSLKAEDTAVYYCAKDDSTSAYYYYYYMDVWGKGTTVTVSS

SEQ ID NO: 6 - protein, 112 aa, Homo sapiens
QSALTQPASVSGSPGQSITISCTGTSSDVGSYNVVSWYQQHPGKAPQLMIYEVSKRPSGVSNRFSGSKSGNTASLTISGLQAEDEADYYCCSYAGSSTSWVVFGGGTKLTVL

SEQ ID NO: 7 - protein, 123 aa, Homo sapiens
QVQLQGSGPGLVKPSETLSLTCTVSGGSISSSSYYWGWIRQPPGKGLEWIGSIYYSGSTYYNPSLKSRVTISVDTSKNQFSLKLTSVTAADTAVYYCARHPTFSGYEYYFDHWGQGTLITVSS

SEQ ID NO: 8 - protein, 112 aa, Homo sapiens
EIVLTQSPDSLAVSLGERATINCKSSQSVLYSSNNKNYLAWYQQKPGQSPKLLIYWASTRESGVPDRFSGSGSGTDFTLTISSLQAEDVAVYYCQQYYNSYTFGQGTKLEIK

SEQ ID NO: 9 - protein, 122 aa, Homo sapiens
EVQLVESGGGLVQPGGSLRLSCAASGFTFSSYGMSWVRQAPGKGLEWVSGINNHGGSTYYGDSVKGRFTISRDNSKNTLYLQMNSLRAEDTAVYYCAKSDTAMVPYNWFDPWGQGTLVTVSS

SEQ ID NO: 10 - protein, 107 aa, Homo sapiens
EIVLTQSPATLSLSPGERATLSCRASQSVNNYLAWYQQKPGQAPRLLIYDASHRATDIPARFSGSGSGTDFTLTISSLEPEDFAVYYCQQRSNWPLTFGGGTKVEIK

SEQ ID NO: 11 - protein, 124 aa, Homo sapiens
QVQLQQWGAGLLKPSETLSLTCGFYGGSLNNYFWSWIRQPPGKGLEWIGDINHSGSTNYNPSLKSRVTVSVDTSKNQFSLNLNSVTAADTAVYYCARGLFLVYYGSGLGGFDYWGQGTLVTVSS

SEQ ID NO: 12 - protein, 108 aa, Homo sapiens
  misc_feature (3)..(3): Xaa can be any naturally occurring amino acid
EIXLTQSPATLSVSPGERATLSCRASQSVRSNLAWYQQKPGQAPRLLIYGASTRATGTPARFSGSGSGTEFTLTISSLQSEDFAVYYCQQYNYWPPYTFGQGTKLEIK

SEQ ID NO: 13 - protein, 124 aa, Homo sapiens
EVQLVESGGGVVQPGRSLRLSCAASGFTFSRYGMHWVRQAPGKGLEWVALISYDGSNKYYADSVKGRFTISRDKSKNMVYLQMNSLRAEDTALYYCAKDLEYYTSGSYSLFDYWGQGTLVTVSS

SEQ ID NO: 14 - protein, 107 aa, Homo sapiens
DIVMTQSPSSLSASVGDRVTITCRASQDISSALAWYQQKPGKTPKILIYDASSLESGVPSRFSGSGSGTDFTLTISSLQPEDFATYYCQQFNNYPLTFGGGTKVEIK

SEQ ID NO: 15 - protein, 118 aa, Homo sapiens
EVQLVESGGGVVQPGRSLRLSCAASGFTFSIYAIHWVRQAPGQGLEWVAVISYDGSNKYYADSVRGRFTISRDNSKNTLYLQMNTLRAEDTAVYYCARGPTYSYMDVWGKGTTVTVSS

SEQ ID NO: 16 - protein, 109 aa, Homo sapiens
EIVLTQSPGTLSLSPGERATLSCRASQSVSSTYLAWYQQKPAQAPRLLIYGASNRATGIPDRFSGSGSGTDFTLTISRLEPEDFAVYYCQQYGSSPPLTFGPGTKVDIK

SEQ ID NO: 17 - protein, 120 aa, Homo sapiens
EVQLLESGGGLVQPGGSLRLSCAASGFTFSYYALSWVRQAPGKGLEWVSATYGSGSNTYYADSVKGRFTISRDNSKNTLYLQMSNLRADDTAVYYCAKDQRNAYDSFDFWGQGTLVTVSS

SEQ ID NO: 18 - protein, 107 aa, Homo sapiens
DIVMTQSPSSLSASVGDRVTITCQASQDISNYLNCYQQKPGKAPKFLIYDASNLETGVPSRFSGSGSGTDFTFTISSLQPEDIATYYCQQYNNLPLTFGGGTKVEIK

SEQ ID NO: 19 - protein, 128 aa, Homo sapiens
EVQLLESGGGLVQPGRSLRLSCTASGFTFGDYAMIWFRQAPGKGLEWVGFIRSKAYGGTTEYAASMKGRFTISRDDSKSIAYLQMNSLKTEDTAVYYCTRDLDYYDSSGYYPTYIDYWGQGTLVTVSS

SEQ ID NO: 20 - protein, 110 aa, Homo sapiens
QLVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSVVLFGGGTKLTVL

SEQ ID NO: 21 - protein, 129 aa, Homo sapiens
EVQLVESGPGLVKPSQTLSLTCTVSGGSISSGNYYWSWIRQPAGKGLEWIGRIYTSGSTNYNPSLKSRVTISVDTSKNQFSLKLSSVTAADTALYYCARDAYYDFLSGYIPTYNWFDPWGQGTLVTVSS

SEQ ID NO: 22 - protein, 112 aa, Homo sapiens
QSALTQPPSVSGAPGQRVTISCTGSGSNIGAGYDVHWYQQLPGTAPKLLIYGNNNRPSGVPDRFSGSKSGTSASLAITGLQADDEADYYCQSYDSSLSGPVVIGGGTKLTVL

SEQ ID NO: 23 - protein, 124 aa, Homo sapiens
QVQLQQSGPGLVKPSQTLSLTCTVAGGSISSGTYYWSWIRQPAGKGLEWIGRIYTSGSANYNPSLKSRVTISVDTSKNQFSLRLSSVTAEDTAVYYCAREYSSSYYYFYYMDVWGKGTTVTVSS

SEQ ID NO: 24 - protein, 107 aa, Homo sapiens
AIRMTQSPSSLSASVGDRVTITCQASQDISNYLNWYQQKPGKAPKLLIYVASNLETGVPSRFSGSGFGTDFTFTISSLQPEDIATYYCQQFDNLPYTFGQGTKLEIK

SEQ ID NO: 25 - protein, 127 aa, Homo sapiens
EVQLVESGGGVVQPGRSLRLSCAVSGFTFSNYGMHWVRQAPGKGLEWVAVILYDGSNRYYADSVKGRFTVSRDNSKNTLYLQMNSLRAEDTAVYYCAKGGGPYCGGGSCWAHYFDYWGQGTLVSVSS

SEQ ID NO: 26 - protein, 107 aa, Homo sapiens
DIVMTQSPSSLSTSVGDRVTITCQASQDISKYLNWYQQKPGKAPKLLIYDASNLETGVPSRFSGSGSGTDFTFTISSLQPEDIATYYCQQYDNLPTTFGQGTRLEIK

SEQ ID NO: 27 - protein, 112 aa, Homo sapiens
QVQLQQWGPGLVKPSETLSLTCTVSGDSISNYYWSWIRQPPEKGLEWIGYIYYSGSTNYNPSLKSRVTISVDTSKNQFSLKLSSVTAADTAMYYCARDFSLWGQGTLVTVSS

SEQ ID NO: 28 - protein, 108 aa, Homo sapiens
DIVMTQSPGTLSLSPGERATLSCRASQSVSSIYLAWYQQKPGQAPRLLIYSTSSRAVGIPDRFSGGGSGTDFTLTISRLEPEDFAVYYCHQYGSSPWTFGQGTKVEIK

SEQ ID NO: 29 - protein, 125 aa, Homo sapiens
EVQLVESGGGLVKPGGSLRLSCAASGFIFSRYSMNWVRQAPGKGLEWVSSISSSTSFIYYADSVRGRFTISRDNAKNSLYLQMNSLRAEDTAVYYCARWIGGDSSGYYPDAFDIWGQGTMVTVSS

SEQ ID NO: 30 - protein, 108 aa, Homo sapiens
EIVMTQSPVSLSVSPGERATLSCRASQSVSSNLAWYQQKPGQAPRLLIYGASTRATGIPARFSGSGSGTEFTLTISSLQSEDFAVYYCQQYYNWPPWTFGQGTKVEIK

SEQ ID NO: 31 - protein, 122 aa, Homo sapiens
QVQLQQSGPGLVKPSETLSLTCTVSGDSISSYYWSWIRQPPGKGLEWIGYIYYTGSTNYNPSLKSRVTISVDTSKNQFSLKLSSVTAADTAVYYCANLGYNSGWYGGYFEYWGQGTLVTVSS

SEQ ID NO: 32 - protein, 108 aa, Homo sapiens
DIVMTQSPGTLSLSPGERATLSCRASQSVSSNYLAWYQQKPGQAPRLLIYGASSRATGIPDRFSGSGSGTDFILTIRRLEPEDFAVYYCQQYGSSLYTFGQGTKLEIK

SEQ ID NO: 33 - protein, 121 aa, Homo sapiens
QVQLQESGGGVVQPGRSLRLSCAASGFTFSSYAIHWVRQAPGKGLEWVAVISYDGINKYYADSVKGRFTSSRDNSKNTLYLQMNSLRAEDTAVYFCARMYSGSYLGYFDYWGQGTLVTVSS

SEQ ID NO: 34 - protein, 108 aa, Homo sapiens
EIVLTQSPATLSVSPGERATLSCRASQSVSSNLAWYQQKPGQAPRLLIYGASTRATGIPARFSGSGSGTEFTLTISSLQSEDFAVYYCQQYNKWPPITFGQGTRLEIK

SEQ ID NO: 35 - protein, 125 aa, Homo sapiens
EVQLVESGAEVKKPGESLKISCKGSGYTFTRYWIGWVRQMPGKGLEWMGIIYPGDSDTRYSPSFQGHVTISADKSISTAYLQWNSLKASDTAMYYCARLPQYCSNGVCQRWFDPWGQGTLVTVSS

SEQ ID NO: 36 - protein, 107 aa, Homo sapiens
EIVLTQSPSSVSASVGDRVTITCRASQGISSWLAWYQQKPGKAPKLLIYAASSLQSGVPSRFSGSGSGTDFTLTISSLQPEDFATYYCQQGNSFPYTFGQGTNLEIK

SEQ ID NO: 37 - protein, 123 aa, Homo sapiens
EVQLVESGAEVRKPGSSVKVSCKASGGTFSNYTINWVRQAPGQGLEWMGRVIPIFGITNYAQKFQGRVTITADKSTSTAYMQLSSLTSEDTAVYYCARGWWFGELETYYFDYRGQGTLVTVSS

SEQ ID NO: 38 - protein, 113 aa, Homo sapiens
QSALTQPPSVSGAPGQRVTISCTGSSSNIGAGYDVHWYQQLPGTAPKLLIYDNNNRPSGVPDRFSGSKSGTSASLAITGLQAEDEADYYCQSYDSSLSGSHVVFGGGTKLTVL

SEQ ID NO: 39 - protein, 119 aa, Homo sapiens
QVQLQQSGPGLVKPSETLSLTCTVSGYSLNSGYFWDWIRQPPGKGLEWIGSMYHSGSTYYNPSLKTRVTISIDTSKNHFSLKMKSVTAADTAVYYCANKLVPTAPFDYWGQGTLVTVSS

SEQ ID NO: 40 - protein, 107 aa, Homo sapiens
NFMLTQPPSVSVSPGQTASITCPGDKLGDKFACWYQQKPGQSPVVVIYQDNKRPSGIPERFSGSNSGNTATLTISGTQAMDEADYYCQAWHSSTVVFGGGTKLTVLG

SEQ ID NO: 41 - protein, 124 aa, Homo sapiens
QVQLQESGGGVVQPGRSLRLSCAASGFTFSTYGMHWVRQAPGKGLEWVAVISYDGSNKYYADSVKGRFTISRDNSKNTLYLQMNSLRAEDTAVYYCAKDPHIVVVPAAMRFEAWGQGTLVTVSS

SEQ ID NO: 42 - protein, 112 aa, Homo sapiens
QSALTQPASVSGSPGQSITISCSGTSSDVGRYNYVSWYQQRPGKAPKLMIYDVSDRPSGVSHRFSGSKSGNTASLTISGLQAEDEADYYCTSHTSSTISYVVFGGGTKLTVL

SEQ ID NO: 43 - protein, 127 aa, Homo sapiens
QVQLQESGPGLVKPSETLSLTCTVSGGSISSYYWSWIRQPPGKGLEWIGYIYYSGSTNYNPSLKSRVTISADTSKNQFSLKLSSVTAADTAVYYCARAPGATYSSGWYYYYYYMDVWGKGTTVTVSS

SEQ ID NO: 44 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIK

SEQ ID NO: 45 - protein, 122 aa, Homo sapiens
EVQLVESGGGVVQPGGSLRLSCAASGFPFRNYAMHWVRQAPGKGLEHISVISSRGDTTYYANSVKGRFTISRDNSQNTLYLQMDSLRADDMAVYYCARVQSGFSYGYGFDYWGQGTLVTVSS

SEQ ID NO: 46 - protein, 112 aa, Homo sapiens
EIVLTQSPLSLPVTPGEPASISCRSSQSLLHINGYNYLDWYLQKPGQSPQLLIYLGSNRASGVPDRFSGSGSGTDFTLKISRVEAEDVGVYYCMQALQTPWTFGQGTKVEIK

SEQ ID NO: 47 - protein, 125 aa, Homo sapiens
EVQLLESGGGLVQPGGSLRLSCAASGFTFSSYAMSWVRQAPGKGLEWVSTISGSGGSTYYADSVKGRFTISDNSKNTLYLQMNSLRAEDTAVYFCAKDGAVATGPGYFYFYMDVWGKGTTVTVSS

SEQ ID NO: 48 - protein, 112 aa, Homo sapiens
QPVLTQPASVSGSPGQSITISCTGTSSDVGSYNLVSWYQQHPGKAPKLMIYEVSKRPSGVSNRFSGSKSGNTASLTISGLQAEDEADYYCCSYAGSSTSYVVFGGGTKLTVL

SEQ ID NO: 49 - protein, 124 aa, Homo sapiens
EVQLVESGGGVVQPGRSLRLSCAASGFTFSSYDMHWVRQAPGKGLEWVAFISYDGSNKYYADPVRGRFTISRDNSKNTLYLQMNSLRAEDTAVYYCAKDLEYYGSGSYSLFDYWGQGTLVTVSS

SEQ ID NO: 50 - protein, 107 aa, Homo sapiens
EIVLTQSPSSLSTSVGDRVTITCRASQGISSALAWYQQKPGKTPKLLIYDASSLESGVPSRFSGSGSGTDFTLTISSLQPEDFATYYCQQFNNYPLTFGGGTKVEIK

SEQ ID NO: 51 - protein, 120 aa, Homo sapiens
EVQLVESGGGVVQPGRSLRLSCAASGFTFSSYAIYWVRQAPGKGLEWVAVISYDGSNKYYADSVRGRFTISRDNSKNTLYLQMNSLRAEDTAVYYCARGGGGYNTFFDYWGQGTLVTVSS

SEQ ID NO: 52 - protein, 113 aa, Homo sapiens
AIRMTQTPDSLAVSLGERATINCKSSQSVLYSSNNKNYLAWYQQKPGQPPKLLIYWASTRESGVPDRFSGSGSGTDFTLTISSLQAEDVAVYFCQQYYSTPLTFGGGTKVEIK

SEQ ID NO: 53 - protein, 126 aa, Homo sapiens
EVQLLESGPGLVKPSETLSLTCTVSGGSIRSYSHYWGWIRQPPGKGLEWIGTIYYSGSTYYNPSLKSRITISVDTSKSQFSLKLSSVTAADTAVYFCASTIPTYDDILTGYQFDYWGQGTLVTVSS

SEQ ID NO: 54 - protein, 103 aa, Homo sapiens
EIVLTQSPSSLSASVGDRVTITCQASQDISNFLNWYQQKPGKAPKLLIYDASNLETRVPSRFSGSGSGTDFTFTISSLQPEDIATYYCQHYTTFGGGTKVEIK

SEQ ID NO: 55 - protein, 119 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARVGLSSWYFEYWGQGTLVTVSS

SEQ ID NO: 56 - protein, 106 aa, Homo sapiens
AQSALTQPPSVSVSPGQTASITCSGDKLGDKYVCWYQQKPGQSPVLVIYQDTKRPSGIPERFSGSNSGNTATLTISGTQTMDEADYYCQAWDSSTGVFGGGTKLTV

SEQ ID NO: 57 - protein, 134 aa, Homo sapiens
EVQLVESGAEVKKPGASVKVSCKTSGYIFTDYFMHWVRQAPGQGLEWMGWINPNSGGANYAQKFQGRVTMTRDTSITTVYMELSRLRIDDTAMYYCAREASLNRSRYYSSGGTVYYYYYYMDVWGKGTTVTVSS

SEQ ID NO: 58 - protein, 110 aa, Homo sapiens
QSVLTQPASVSGSPGQSITISCTGSSSDVGGYDFVSWYQQHPGKAPKVVIYDVTNRPSGVSNRFSGSKSGNTASLTISGLQAEDEADYYCSSYTSSSTRVFGTGTKVTVL

SEQ ID NO: 59 - protein, 127 aa, Homo sapiens
EVQLVESGPGQVKPSETLSLTCTVSGYSISSGYFWGWVRQPPGKGLEWIGSIYHSGSTYYNPSLKSRVTISVDTSKNQFSLNLSSVTAADTAVYYCARDHGSYDFWSGYSRDAFDIWGQGTMVTFSS

SEQ ID NO: 60 - protein, 107 aa, Homo sapiens
DIVMTQSPSSLSASVGDRVTITCRASQGIRNDLGWYQQKPGKAPKRLIYAASSLQSGVPSRFSGSGSGTEFTLTISSLQPEDFATYYCLQYNSYPRTFGQGTKVEIK

SEQ ID NO: 61 - protein, 115 aa, Homo sapiens
EVQLVESGGGLIQPGGSLRLSCAASGFSVSSNYMNWVRQAPRKGLEWVSVIYSGGSTYYADSVKGRFTISRDNSKNTLYLQMNTLRAEDTAVYYCARGYGDSQRWGQGTLVTVSS

SEQ ID NO: 62 - protein, 107 aa, Homo sapiens
DIGMTQSPSTLSASVGDRVTITCRASQSISSWLAWYQHKPGKAPKLLIYKASSLESGVPSRFSGSGSGTEFTLTISSLQPDDFATYYCQQYNTYSFTFGPGTKVDPK

SEQ ID NO: 63 - protein, 122 aa, Homo sapiens
QVQLQESGPGLVKPSETLSLTCTVSGGSVSSDNYYWSWIRQPPGKGLEWIGYIYYSGSTNYNPSLKSRVTMSVDTSKNQFSLKLSSVTAADTAVYYCARGFVATYYYYMDVWGKGTTVTVSS

SEQ ID NO: 64 - protein, 106 aa, Homo sapiens
DIVMTQSPSSLSASIGDRVTITCRASQSINNYLNWYQQKPGKAPELLIYAASSLQSGVPSRFSGSGSGTDFTLTISSLQPEDFATYYCQQSYSPYTFGQGTKLEIK

SEQ ID NO: 65 - protein, 15 aa, Severe acute respiratory syndrome coronavirus 2 (SARS-CoV-2)
VNNTVYDPLQPELDS

SEQ ID NO: 66 - protein, 15 aa, Severe acute respiratory syndrome coronavirus 2 (SARS-CoV-2)
VYDPLQPELDSFKEE

SEQ ID NO: 67 - protein, 15 aa, Severe acute respiratory syndrome coronavirus 2 (SARS-CoV-2)
LQPELDSFKEELDKY

SEQ ID NO: 68 - protein, 15 aa, Severe acute respiratory syndrome coronavirus 2 (SARS-CoV-2)
LDSFKEELDKYFKNH

SEQ ID NO: 69 - protein, 15 aa, Severe acute respiratory syndrome coronavirus 2 (SARS-CoV-2)
KEELDKYFKNHTSPD

SEQ ID NO: 70 - protein, 15 aa, Severe acute respiratory syndrome coronavirus 2 (SARS-CoV-2)
DKYFKNHTSPDVDLG

SEQ ID NO: 71 - protein, 15 aa, Severe acute respiratory syndrome coronavirus 2 (SARS-CoV-2)
KNHTSPDVDLGDISG

SEQ ID NO: 72 - protein, 15 aa, Human immunodeficiency virus type 1
NMWKNDMVDQMHEDI

SEQ ID NO: 73 - protein, 23 aa, Severe acute respiratory syndrome coronavirus (SARS-CoV)
LDSFKEELDKYFKNHTSPDVDLG

SEQ ID NO: 74 - protein, 25 aa, Middle East respiratory syndrome coronavirus (MERS-CoV)
IDFQDELDEFFKNVSTSIPNFGSLT

SEQ ID NO: 75 - protein, 23 aa, Human coronavirus OC43 (HCoV-OC43)
LPDFKEELDQWFKNQTSVAPDLS

SEQ ID NO: 76 - protein, 24 aa, Human coronavirus HKU1 (HCoV-HKU1)
LSDFESELSHWFKNQTSIAPNLTL

SEQ ID NO: 77 - protein, 7 aa, Homo sapiens
GFTFTSS

SEQ ID NO: 78 - protein, 6 aa, Homo sapiens
VVGSGN

SEQ ID NO: 79 - protein, 14 aa, Homo sapiens
PSCSGGRCYDGFDI

SEQ ID NO: 80 - protein, 12 aa, Homo sapiens
RASQSVSSSYLA

SEQ ID NO: 81 - protein, 7 aa, Homo sapiens
GASSRAT

SEQ ID NO: 82 - protein, 9 aa, Homo sapiens
QQYGTSPWT

SEQ ID NO: 83 - protein, 7 aa, Homo sapiens
GFTFSDY

SEQ ID NO: 84 - protein, 6 aa, Homo sapiens
GSSGSS

SEQ ID NO: 85 - protein, 11 aa, Homo sapiens
DGSYGDYVRGY

SEQ ID NO: 86 - protein, 11 aa, Homo sapiens
RASQTISSWLA

SEQ ID NO: 87 - protein, 7 aa, Homo sapiens
KASTLES

SEQ ID NO: 88 - protein, 9 aa, Homo sapiens
QQYNSYPWT

SEQ ID NO: 89 - protein, 7 aa, Homo sapiens
GFTFSSY

SEQ ID NO: 90 - protein, 6 aa, Homo sapiens
SGSGGS

SEQ ID NO: 91 - protein, 15 aa, Homo sapiens
DDSTSAYYYYYYMDV

SEQ ID NO: 92 - protein, 14 aa, Homo sapiens
TGTSSDVGSYNVVS

SEQ ID NO: 93 - protein, 7 aa, Homo sapiens
EVSKRPS

SEQ ID NO: 94 - protein, 12 aa, Homo sapiens
CSYAGSSTSWVV

SEQ ID NO: 95 - protein, 9 aa, Homo sapiens
GGSISSSSY

SEQ ID NO: 96 - protein, 5 aa, Homo sapiens
YYSGS

SEQ ID NO: 97 - protein, 13 aa, Homo sapiens
HPTFSGYEYYFDH

SEQ ID NO: 98 - protein, 17 aa, Homo sapiens
KSSQSVLYSSNNKNYLA

SEQ ID NO: 99 - protein, 7 aa, Homo sapiens
WASTRES

SEQ ID NO: 100 - protein, 8 aa, Homo sapiens
QQYYNSYT

SEQ ID NO: 101 - protein, 6 aa, Homo sapiens
NNHGGS

SEQ ID NO: 102 - protein, 13 aa, Homo sapiens
SDTAMVPYNWFDP

SEQ ID NO: 103 - protein, 7 aa, Homo sapiens
GFTFSRY

SEQ ID NO: 104 - protein, 6 aa, Homo sapiens
SYDGSN

SEQ ID NO: 105 - protein, 15 aa, Homo sapiens
DLEYYTSGSYSLFDY

SEQ ID NO: 106 - protein, 11 aa, Homo sapiens
RASQDISSALA

SEQ ID NO: 107 - protein, 7 aa, Homo sapiens
DASSLES

SEQ ID NO: 108 - protein, 9 aa, Homo sapiens
QQFNNYPLT

SEQ ID NO: 109 - protein, 7 aa, Homo sapiens
GFTFSIY

SEQ ID NO: 110 - protein, 9 aa, Homo sapiens
GPTYSYMDV

SEQ ID NO: 111 - protein, 12 aa, Homo sapiens
RASQSVSSTYLA

SEQ ID NO: 112 - protein, 7 aa, Homo sapiens
GASNRAT

SEQ ID NO: 113 - protein, 10 aa, Homo sapiens
QQYGSSPPLT

SEQ ID NO: 114 - protein, 7 aa, Homo sapiens
GFTFSYY

SEQ ID NO: 115 - protein, 6 aa, Homo sapiens
YGSGSN

SEQ ID NO: 116 - protein, 11 aa, Homo sapiens
DQRNAYDSFDF

SEQ ID NO: 117 - protein, 11 aa, Homo sapiens
QASQDISNYLN

SEQ ID NO: 118 - protein, 7 aa, Homo sapiens
DASNLET

SEQ ID NO: 119 - protein, 9 aa, Homo sapiens
QQYNNLPLT

SEQ ID NO: 120 - protein, 7 aa, Homo sapiens
GFTFGDY

SEQ ID NO: 121 - protein, 8 aa, Homo sapiens
RSKAYGGT

SEQ ID NO: 122 - protein, 17 aa, Homo sapiens
DLDYYDSSGYYPTYIDY

SEQ ID NO: 123 - protein, 13 aa, Homo sapiens
SGSSSNIGNNYVS

SEQ ID NO: 124 - protein, 7 aa, Homo sapiens
DNNKRPS

SEQ ID NO: 125 - protein, 11 aa, Homo sapiens
GTWDSSLSVVL

SEQ ID NO: 126 - protein, 9 aa, Homo sapiens
GGSISSGNY

SEQ ID NO: 127 - protein, 5 aa, Homo sapiens
YTSGS

SEQ ID NO: 128 - protein, 14 aa, Homo sapiens
EYSSSYYYFYYMDV

SEQ ID NO: 129 - protein, 7 aa, Homo sapiens
GFTFSNY

SEQ ID NO: 130 - protein, 6 aa, Homo sapiens
LYDGSN

SEQ ID NO: 131 - protein, 18 aa, Homo sapiens
GGGPYCGGGSCWAHYFDY

SEQ ID NO: 132 - protein, 11 aa, Homo sapiens
QASQDISKYLN

SEQ ID NO: 133 - protein, 9 aa, Homo sapiens
QQYDNLPTT

SEQ ID NO: 134 - protein, 7 aa, Homo sapiens
GDSISNY

SEQ ID NO: 135 - protein, 4 aa, Homo sapiens
DFSL

SEQ ID NO: 136 - protein, 12 aa, Homo sapiens
RASQSVSSIYLA

SEQ ID NO: 137 - protein, 7 aa, Homo sapiens
STSSRAV

SEQ ID NO: 138 - protein, 9 aa, Homo sapiens
HQYGSSPWT

SEQ ID NO: 139 - protein, 7 aa, Homo sapiens
GFIFSRY

SEQ ID NO: 140 - protein, 6 aa, Homo sapiens
SSSTSF

SEQ ID NO: 141 - protein, 16 aa, Homo sapiens
WIGGDSSGYYPDAFDI

SEQ ID NO: 142 - protein, 11 aa, Homo sapiens
RASQSVSSNLA

SEQ ID NO: 143 - protein, 7 aa, Homo sapiens
GASTRAT

SEQ ID NO: 144 - protein, 10 aa, Homo sapiens
QQYYNWPPWT

SEQ ID NO: 145 - protein, 7 aa, Homo sapiens
GDSISSY

SEQ ID NO: 146 - protein, 5 aa, Homo sapiens
YYTGS

SEQ ID NO: 147 - protein, 14 aa, Homo sapiens
LGYNSGWYGGYFEY

SEQ ID NO: 148 - protein, 6 aa, Homo sapiens
SYDGIN

SEQ ID NO: 149 - protein, 12 aa, Homo sapiens
MYSGSYLGYFDY

SEQ ID NO: 150 - protein, 10 aa, Homo sapiens
QQYNKWPPIT

SEQ ID NO: 151 - protein, 7 aa, Homo sapiens
GGTFSNY

SEQ ID NO: 152 - protein, 6 aa, Homo sapiens
IPIFGI

SEQ ID NO: 153 - protein, 14 aa, Homo sapiens
GWWFGELETYYFDY

SEQ ID NO: 154 - protein, 14 aa, Homo sapiens
TGSSSNIGAGYDVH

SEQ ID NO: 155 - protein, 7 aa, Homo sapiens
DNNNRPS

SEQ ID NO: 156 - protein, 13 aa, Homo sapiens
QSYDSSLSGSHVV

SEQ ID NO: 157 - protein, 8 aa, Homo sapiens
GYSLNSGY

SEQ ID NO: 158 - protein, 5 aa, Homo sapiens
YHSGS

SEQ ID NO: 159 - protein, 10 aa, Homo sapiens
KLVPTAPFDY

SEQ ID NO: 160 - protein, 11 aa, Homo sapiens
PGDKLGDKFAC

SEQ ID NO: 161 - protein, 7 aa, Homo sapiens
QDNKRPS

SEQ ID NO: 162 - protein, 9 aa, Homo sapiens
QAWHSSTVV

SEQ ID NO: 163 - protein, 7 aa, Homo sapiens
GFTFSTY

SEQ ID NO: 164 - protein, 15 aa, Homo sapiens
DPHIVVVPAAMRFEA

SEQ ID NO: 165 - protein, 14 aa, Homo sapiens
SGTSSDVGRYNYVS

SEQ ID NO: 166 - protein, 7 aa, Homo sapiens
DVSDRPS

SEQ ID NO: 167 - protein, 12 aa, Homo sapiens
TSHTSSTISYVV

SEQ ID NO: 168 - protein, 7 aa, Homo sapiens
GGSISSY

SEQ ID NO: 169 - protein, 19 aa, Homo sapiens
APGATYSSGWYYYYYYMDV

SEQ ID NO: 170 - protein, 16 aa, Homo sapiens
RSSQSLLHSNGYNYLD

SEQ ID NO: 171 - protein, 7 aa, Homo sapiens
LGSNRAS

SEQ ID NO: 172 - protein, 9 aa, Homo sapiens
MQALQTPFT

SEQ ID NO: 173 - protein, 16 aa, Homo sapiens
RSSQSLLHINGYNYLD

SEQ ID NO: 174 - protein, 7 aa, Homo sapiens
GFPFRNY

SEQ ID NO: 175 - protein, 6 aa, Homo sapiens
SSRGDT

SEQ ID NO: 176 - protein, 13 aa, Homo sapiens
VQSGFSYGYGFDY

SEQ ID NO: 177 - protein, 9 aa, Homo sapiens
MQALQTPWT

SEQ ID NO: 178 - protein, 17 aa, Homo sapiens
DGAVATGPGYFYFYMDV

SEQ ID NO: 179 - protein, 14 aa, Homo sapiens
TGTSSDVGSYNLVS

SEQ ID NO: 180 - protein, 12 aa, Homo sapiens
CSYAGSSTSYVV

SEQ ID NO: 181 - protein, 15 aa, Homo sapiens
DLEYYGSGSYSLFDY

SEQ ID NO: 182 - protein, 11 aa, Homo sapiens
RASQGISSALA

SEQ ID NO: 183 - protein, 11 aa, Homo sapiens
GGGGYNTFFDY

SEQ ID NO: 184 - protein, 9 aa, Homo sapiens
GGSIRSYSH

SEQ ID NO: 185 - protein, 16 aa, Homo sapiens
TIPTYDDILTGYQFDY

SEQ ID NO: 186 - protein, 11 aa, Homo sapiens
QASQDISNFLN

SEQ ID NO: 187 - protein, 5 aa, Homo sapiens
QHYTT

SEQ ID NO: 188 - protein, 6 aa, Homo sapiens
KQDGSE

SEQ ID NO: 189 - protein, 10 aa, Homo sapiens
VGLSSWYFEY

SEQ ID NO: 190 - protein, 11 aa, Homo sapiens
SGDKLGDKYVC

SEQ ID NO: 191 - protein, 7 aa, Homo sapiens
QDTKRPS

SEQ ID NO: 192 - protein, 9 aa, Homo sapiens
QAWDSSTGV

SEQ ID NO: 193 - protein, 7 aa, Homo sapiens
GYIFTDY

SEQ ID NO: 194 - protein, 6 aa, Homo sapiens
NPNSGG

SEQ ID NO: 195 - protein, 25 aa, Homo sapiens
EASLNRSRYYSSGGTVYYYYYYMDV

SEQ ID NO: 196 - protein, 14 aa, Homo sapiens
TGSSSDVGGYDFVS

SEQ ID NO: 197 - protein, 7 aa, Homo sapiens
DVTNRPS

SEQ ID NO: 198 - protein, 10 aa, Homo sapiens
SSYTSSSTRV

SEQ ID NO: 199 - protein, 8 aa, Homo sapiens
GYSISSGY

SEQ ID NO: 200 - protein, 18 aa, Homo sapiens
DHGSYDFWSGYSRDAFDI

SEQ ID NO: 201 - protein, 7 aa, Homo sapiens
GFSVSSN

SEQ ID NO: 202 - protein, 5 aa, Homo sapiens
YSGGS

SEQ ID NO: 203 - protein, 7 aa, Homo sapiens
GYGDSQR

SEQ ID NO: 204 - protein, 9 aa, Homo sapiens
GGSVSSDNY

SEQ ID NO: 205 - protein, 12 aa, Homo sapiens
GFVATYYYYMDV

SEQ ID NO: 206 - protein, 11 aa, Homo sapiens
RASQSINNYLN

SEQ ID NO: 207 - protein, 7 aa, Homo sapiens
AASSLQS

SEQ ID NO: 208 - protein, 8 aa, Homo sapiens
QQSYSPYT

SEQ ID NO: 209 - protein, 7 aa, Homo sapiens
GYTFTRY

SEQ ID NO: 210 - protein, 6 aa, Homo sapiens
YPGDSD

SEQ ID NO: 211 - protein, 16 aa, Homo sapiens
LPQYCSNGVCQRWFDP

SEQ ID NO: 212 - protein, 11 aa, Homo sapiens
RASQGISSWLA

SEQ ID NO: 213 - protein, 9 aa, Homo sapiens
QQGNSFPYT

SEQ ID NO: 214 - protein, 11 aa, Homo sapiens
RASQSVNNYLA

SEQ ID NO: 215 - protein, 7 aa, Homo sapiens
DASHRAT

SEQ ID NO: 216 - protein, 9 aa, Homo sapiens
QQRSNWPLT

SEQ ID NO: 217 - protein, 11 aa, Homo sapiens
RASQSVRSNLA

SEQ ID NO: 218 - protein, 10 aa, Homo sapiens
QQYNYWPPYT

SEQ ID NO: 219 - protein, 7 aa, Homo sapiens
GGSLNNY

SEQ ID NO: 220 - protein, 5 aa, Homo sapiens
NHSGS

SEQ ID NO: 221 - protein, 16 aa, Homo sapiens
GLFLVYYGSGLGGFDY

SEQ ID NO: 222 - protein, 14 aa, Homo sapiens
TGSGSNIGAGYDVH

SEQ ID NO: 223 - protein, 7 aa, Homo sapiens
GNNNRPS

SEQ ID NO: 224 - protein, 12 aa, Homo sapiens
QSYDSSLSGPVV

SEQ ID NO: 225 - protein, 19 aa, Homo sapiens
DAYYDFLSGYIPTYNWFDP

SEQ ID NO: 226 - protein, 7 aa, Homo sapiens
VASNLET

SEQ ID NO: 227 - protein, 9 aa, Homo sapiens
QQFDNLPYT

SEQ ID NO: 228 - protein, 9 aa, Homo sapiens
GGSISSGTY

SEQ ID NO: 229 - protein, 12 aa, Homo sapiens
RASQSVSSNYLA

SEQ ID NO: 230 - protein, 9 aa, Homo sapiens
QQYGSSLYT

SEQ ID NO: 231 - protein, 9 aa, Homo sapiens
QQYYSTPLT

SEQ ID NO: 232 - protein, 11 aa, Homo sapiens
RASQGIRNDLG

SEQ ID NO: 233 - protein, 9 aa, Homo sapiens
LQYNSYPRT

SEQ ID NO: 234 - protein, 11 aa, Homo sapiens
RASQSISSWLA

SEQ ID NO: 235 - protein, 7 aa, Homo sapiens
KASSLES

SEQ ID NO: 236 - protein, 9 aa, Homo sapiens
QQYNTYSFT

(19) United States
(12) Patent Application Publication
(10) Pub. No.: US 2024/0400651 A1
(43) Pub. Date: Dec. 5, 2024

(54) NEUTRALIZING MONOCLONAL ANTIBODIES AGAINST COVID-19

(71) Applicants: VAL-CHUM, LIMITED PARTNERSHIP, Québec, Québec (CA); FRED HUTCHINSON CANCER CENTER, Seattle, WA (US)

(72) Inventors: Leonidas STAMATATOS, Seattle, WA (US); Andrew MCGUIRE, Seattle, WA (US); Marie PANCERA, Seattle, WA (US); Andrés FINZI, Saint-Bruno-de-Montarville, Québec (CA)

(21) Appl. No.: 18/259,748

(22) PCT Filed: Dec. 22, 2021
(86) PCT No.: PCT/CA2021/051873

§ 371 Date: Jun. 28, 2023

Related U.S. Application Data
(60) Provisional application No. 63/131,608, filed on Dec. 29, 2020.
(60) Provisional application No. 63/260,285, filed on Aug. 16, 2021.

(51) Int. Cl. C07K 16/10; A61K 39/00; A61P 31/14

(52) U.S. Cl. CPC C07K 16/1003; A61P 31/14; A61K 2039/505

(57) ABSTRACT

The present application relates to neutralizing antibodies or antigen-binding fragments thereof against betacoronaviruses such as severe acute respiratory syndrome coronavirus 2 (SARS-CoV-2), to nucleic acid(s) encoding such neutralizing antibodies or antigen-binding fragments thereof, and to mixture and compositions comprising such antibodies, antigen-binding fragments or nucleic acids. Methods and uses of the antibodies, antigen-binding fragments thereof, nucleic acid(s) or compositions, including therapeutic, diagnostic, and preventative methods and uses for betacoronavirus infections and related diseases such as COVID-19, are also described.

### CROSS REFERENCE TO RELATED APPLICATIONS

This application is the U.S. national phase of International Application No. PCT/CA2021/051873, filed Dec. 22, 2021, which designated the U.S. and claims the benefit of U.S. provisional application Ser. No. 63/131,608, filed on Dec. 29, 2020, and U.S. provisional application Ser. No. 63/260,285, filed on Aug. 16, 2021, each of which are incorporated herein by reference in their entirety.

### REFERENCE TO SEQUENCE LISTING SUBMITTED ELECTRONICALLY

The content of the electronically submitted sequence listing (Name: 8819-3_Sequence_Listing.txt; Size: 96,708 bytes; and Date of Creation: Jul. 16, 2024) filed on Aug. 15, 2024, is incorporated herein by reference in its entirety.

### TECHNICAL FIELD

The present disclosure generally relates to viral infection, and more particularly to the prevention and/or treatment of coronavirus infection such as severe acute respiratory syndrome coronavirus 2 (SARS-CoV-2) infection.

### BACKGROUND ART

Coronaviruses are large, roughly spherical, RNA viruses with bulbous surface projections that cause diseases in mammals and birds. In humans, these viruses cause respiratory tract infections that can range from mild to lethal. Mild illnesses include some cases of the common cold (which is also caused by other viruses, predominantly rhinoviruses), while more lethal varieties can cause severe acute respiratory syndrome (SARS), Middle East respiratory syndrome (MERS), and Coronavirus disease 2019 (COVID-19). Coronaviruses have four structural proteins, namely the Spike (S), Envelope (E), and Membrane (M) proteins, forming the viral envelope, as well as the Nucleocapsid (N) protein, holding the viral RNA genome.

Severe acute respiratory syndrome coronavirus 2 (SARS-CoV-2) is the strain of coronavirus that causes COVID-19, the respiratory illness responsible for the COVID-19 pandemic. The spike protein SARS-CoV-2 is the glycoprotein responsible for allowing the virus to attach to and fuse with the membrane of a host cell; specifically, its S1 subunit contains the receptor-binding domain (RBD) that interacts with the cellular receptor angiotensin-converting enzyme 2 (ACE2) and catalyzes virus attachment, and its S2 subunit possesses the fusion machinery, which can mediate host-viral membrane fusion after S1 shedding. The main receptor involved in SARS-CoV-2 entry into human cells is the angiotensin converting enzyme 2 (ACE2). After attachment of a SARS-CoV-2 virion to a target cell, the cell's protease transmembrane protease, serine 2 (TMPRSS2) cuts open the spike protein of the virus, exposing a fusion peptide in the S2 subunit, and the host receptor ACE2.

Multiple variants of SARS-CoV-2 are circulating globally and within the United States. Four new variants that have rapidly become dominant within their countries have aroused concerns: B.1.1.7 (also known as VOC-202012/01), 501Y.V2 (B.1.351), P.1 (B.1.1.28.1), delta (B.1.617.2) and B.1.1.529 (omicron).

The B.1.1.7 variant (23 mutations with 17 amino acid changes) was first described in the United Kingdom in December 2020; the 501Y.V2 variant (23 mutations with 17 amino acid changes) was initially reported in South Africa in December 2020; and the P.1 variant (approximately 35 mutations with 17 amino acid changes) was reported in Brazil in January 2021. By February 2021, the B.1.1.7 variant had been reported in 93 countries, the 501Y.V2 variant in 45, and the P.1 variant in 21. All three variants have the N501Y mutation, which changes the amino acid asparagine (N) to tyrosine (Y) at position 501 in the receptor-binding domain of the spike protein. The 501Y.V2 and P.1 variants both have two additional receptor-binding-domain mutations, K417N/T and E484K. These mutations increase the binding affinity of the receptor-binding domain to the angiotensin-converting enzyme 2 (ACE2) receptor. Four key concerns stemming from the emergence of the new variants are their effects on viral transmissibility, disease severity, reinfection rates (i.e., escape from natural immunity), and vaccine effectiveness (i.e., escape from vaccine-induced immunity). Recently, two more SARS-CoV-2 variants, B.1.427 and B.1.429, which were first detected in California, have been shown to be approximately 20% more transmissible than pre-existing variants and have been classified by the CDC as variants of concern. The B.1.617.2 delta variant comprises the following substitutions in the Spike protein that are known to affect transmissibility of the virus: D614G, T478K, P681R and L452R. The B.1.1.529 (omicron) variant was reported to the WHO in November 2021 and comprises 32 mutations in the Spike proteins. Studies on these variants have provided compelling evidence that they have the potential to escape naturally-induced immunity as well as the immunity induced by currently approved vaccines.

Current evidence indicates that SARS-CoV-2, the etiologic agent of COVID-19, will become endemic in the population. The current pandemic is aggravated by the apparition of variants of concern that are feared to result in an antigenic drift that could evade vaccine-elicited immune responses.

Thus, there is a need for the development of therapies that elicit neutralizing activity against SARS-CoV-2, including SARS-CoV-2 variants.

The present description refers to a number of documents, the content of which is herein incorporated by reference in their entirety.

### SUMMARY OF THE INVENTION

The present disclosure provides the following items 1 to 47:

1. An antibody or an antigen binding fragment thereof comprises one of the following combinations of complementarity determining regions (CDRs):
(a) a light chain CDR1 (CDR-L1) comprising an amino acid sequence having at least 70% identity with the sequence RASQSVSSSYLA (SEQ ID NO:80); a light chain CDR2 (CDR-L2) comprising the sequence GASSRAT (SEQ ID NO:81); a light chain CDR3 (CDR-L3) comprising the sequence QQYGTSPWT (SEQ ID NO:82); a heavy chain CDR1 (CDR-H1) comprising the sequence GFTFTSS (SEQ ID NO:77); a heavy chain CDR2 (CDR-H2) comprising the sequence VVGSGN (SEQ ID NO:78); and a heavy chain CDR3 (CDR-H3) comprising the sequence PSCSGGRCYDGFDI (SEQ ID NO:79);
(b) a CDR-L1 comprising the sequence RASQGISSWLA (SEQ ID NO:212); a CDR-L2 comprising the sequence AASSLQS (SEQ ID NO:207); a CDR-L3 comprising the sequence QQGNSFPYT (SEQ ID NO:213); a CDR-H1 comprising the sequence GYTFTRY (SEQ ID NO:209); a CDR-H2 comprising the sequence YPGDSD (SEQ ID NO:210); and a CDR-H3 comprising the sequence LPQYCSNGVCQRWFDP (SEQ ID NO:211);
(c) a CDR-L1 comprising the sequence RASQTISSWLA (SEQ ID NO:86); a CDR-L2 comprising the sequence KASTLES (SEQ ID NO:87); a CDR-L3 comprising the sequence QQYNSYPWT (SEQ ID NO:88); a CDR-H1 comprising the sequence GFTFSDY (SEQ ID NO:83); a CDR-H2 comprising the sequence GSSGSS (SEQ ID NO:84); and a CDR-H3 comprising the sequence DGSYGDYVRGY (SEQ ID NO:85);
(d) a CDR-L1 comprising the sequence TGTSSDVGSYNVVS (SEQ ID NO:92); a CDR-L2 comprising the sequence EVSKRPS (SEQ ID NO:93); a CDR-L3 comprising the sequence CSYAGSSTSWVV (SEQ ID NO:94); a CDR-H1 comprising the sequence GFTFSSY (SEQ ID NO:89); a CDR-H2 comprising the sequence SGSGGS (SEQ ID NO:90); and a CDR-H3 comprising the sequence DDSTSAYYYYYYMDV (SEQ ID NO:91);
(e) a CDR-L1 comprising the sequence KSSQSVLYSSNNKNYLA (SEQ ID NO:98); a CDR-L2 comprising the sequence WASTRES (SEQ ID NO:99); a CDR-L3 comprising the sequence QQYYNSYT (SEQ ID NO:100); a CDR-H1 comprising the sequence GGSISSSSY (SEQ ID NO:95); a CDR-H2 comprising the sequence YYSGS (SEQ ID NO:96); and a CDR-H3 comprising the sequence HPTFSGYEYYFDH (SEQ ID NO:97);
(f) a CDR-L1 comprising the sequence RASQSVNNYLA (SEQ ID NO:214); a CDR-L2 comprising the sequence DASHRAT (SEQ ID NO:215); a CDR-L3 comprising the sequence QQRSNWPLT (SEQ ID NO:216); a CDR-H1 comprising the sequence GFTFSSY (SEQ ID NO:89); a CDR-H2 comprising the sequence NNHGGS (SEQ ID NO:101); and a CDR-H3 comprising the sequence SDTAMVPYNWFDP (SEQ ID NO:102);
(g) a CDR-L1 comprising the sequence RASQSVRSNLA (SEQ ID NO:217); a CDR-L2 comprising the sequence GASTRAT (SEQ ID NO:143); a CDR-L3 comprising the sequence QQYNYWPPYT (SEQ ID NO:218); a CDR-H1 comprising the sequence GGSLNNY (SEQ ID NO:219); a CDR-H2 comprising the sequence NHSGS (SEQ ID NO:220); and a CDR-H3 comprising the sequence GLFLVYYGSGLGGFDY (SEQ ID NO:221);
(h) a CDR-L1 comprising the sequence RASQDISSALA (SEQ ID NO:106); a CDR-L2 comprising the sequence DASSLES (SEQ ID NO:107); a CDR-L3 comprising the sequence QQFNNYPLT (SEQ ID NO:108); a CDR-H1 comprising the sequence GFTFSRY (SEQ ID NO:103); a CDR-H2 comprising the sequence SYDGSN (SEQ ID NO:104); and a CDR-H3 comprising the sequence DLEYYTSGSYSLFDY (SEQ ID NO:105);
(i) a CDR-L1 comprising the sequence RASQSVSSTYLA (SEQ ID NO:111); a CDR-L2 comprising the sequence GASNRAT (SEQ ID NO:112); a CDR-L3 comprising the sequence QQYGSSPPLT (SEQ ID NO:113); a CDR-H1 comprising the sequence GFTFSIY (SEQ ID NO:109); a CDR-H2 comprising the sequence SYDGSN (SEQ ID NO:104); and a CDR-H3 comprising the sequence GPTYSYMDV (SEQ ID NO:110);
(j) a CDR-L1 comprising the sequence QASQDISNYLN (SEQ ID NO:117); a CDR-L2 comprising the sequence DASNLET (SEQ ID NO:118); a CDR-L3 comprising the sequence QQYNNLPLT (SEQ ID NO:119); a CDR-H1 comprising the sequence GFTFSYY (SEQ ID NO:114); a CDR-H2 comprising the sequence YGSGSN (SEQ ID NO:115); and a CDR-H3 comprising the sequence DQRNAYDSFDF (SEQ ID NO:116);
(k) a CDR-L1 comprising the sequence SGSSSNIGNNYVS (SEQ ID NO:123); a CDR-L2 comprising the sequence DNNKRPS (SEQ ID NO:124); a CDR-L3 comprising the sequence GTWDSSLSVVL (SEQ ID NO:125); a CDR-H1 comprising the sequence GFTFGDY (SEQ ID NO:120); a CDR-H2 comprising the sequence RSKAYGGT (SEQ ID NO:121); and a CDR-H3 comprising the sequence DLDYYDSSGYYPTYIDY (SEQ ID NO:122);
(l) a CDR-L1 comprising the sequence TGSGSNIGAGYDVH (SEQ ID NO:222); a CDR-L2 comprising the sequence GNNNRPS (SEQ ID NO:223); a CDR-L3 comprising the sequence QSYDSSLSGPVV (SEQ ID NO:224); a CDR-H1 comprising the sequence GGSISSGNY (SEQ ID NO:126); a CDR-H2 comprising the sequence YTSGS (SEQ ID NO:127); and a CDR-H3 comprising the sequence DAYYDFLSGYIPTYNWFDP (SEQ ID NO:225);
(m) a CDR-L1 comprising the sequence QASQDISNYLN (SEQ ID NO:117); a CDR-L2 comprising the sequence VASNLET (SEQ ID NO:226); a CDR-L3 comprising the sequence QQFDNLPYT (SEQ ID NO:227); a CDR-H1 comprising the sequence GGSISSGTY (SEQ ID NO:228); a CDR-H2 comprising the sequence YTSGS (SEQ ID NO:127); and a CDR-H3 comprising the sequence EYSSSYYYFYYMDV (SEQ ID NO:128);
(n) a CDR-L1 comprising the sequence QASQDISKYLN (SEQ ID NO:132); a CDR-L2 comprising the sequence DASNLET (SEQ ID NO:118); a CDR-L3 comprising the sequence QQYDNLPTT (SEQ ID NO:133); a CDR-H1 comprising the sequence GFTFSNY (SEQ ID NO:129); a CDR-H2 comprising the sequence LYDGSN (SEQ ID NO:130); and a CDR-H3 comprising the sequence GGGPYCGGGSCWAHYFDY (SEQ ID NO:131);
(o) a CDR-L1 comprising the sequence RASQSVSSIYLA (SEQ ID NO:136); a CDR-L2 comprising the sequence STSSRAV (SEQ ID NO:137); a CDR-L3 comprising the sequence HQYGSSPWT (SEQ ID NO:138); a CDR-H1 comprising the sequence GDSISNY (SEQ ID NO:134); a CDR-H2 comprising the sequence YYSGS (SEQ ID NO:96); and a CDR-H3 comprising the sequence DFSL (SEQ ID NO:135);
(p) a CDR-L1 comprising the sequence RASQSVSSNLA (SEQ ID NO:142); a CDR-L2 comprising the sequence GASTRAT (SEQ ID NO:143); a CDR-L3 comprising the sequence QQYYNWPPWT (SEQ ID NO:144); a CDR-H1 comprising the sequence GFIFSRY (SEQ ID NO:139); a CDR-H2 comprising the sequence SSSTSF (SEQ ID NO:140); and a CDR-H3 comprising the sequence WIGGDSSGYYPDAFDI (SEQ ID NO:141);
(q) a CDR-L1 comprising the sequence RASQSVSSNYLA (SEQ ID NO:229); a CDR-L2 comprising the sequence GASSRAT (SEQ ID NO:81); a CDR-L3 comprising the sequence QQYGSSLYT (SEQ ID NO:230); a CDR-H1 comprising the sequence GDSISSY (SEQ ID NO:145); a CDR-H2 comprising the sequence YYTGS (SEQ ID NO:146); and a CDR-H3 comprising the sequence LGYNSGWYGGYFEY (SEQ ID NO:147);
(r) a CDR-L1 comprising the sequence RASQSVSSNLA (SEQ ID NO:142); a CDR-L2 comprising the sequence GASTRAT (SEQ ID NO:143); a CDR-L3 comprising the sequence QQYNKWPPIT (SEQ ID NO:150); a CDR-H1 comprising the sequence GFTFSSY (SEQ ID NO:89); a CDR-H2 comprising the sequence SYDGIN (SEQ ID NO:148); and a CDR-H3 comprising the sequence MYSGSYLGYFDY (SEQ ID NO:149);
(s) a CDR-L1 comprising the sequence TGSSSNIGAGYDVH (SEQ ID NO:154); a CDR-L2 comprising the sequence DNNNRPS (SEQ ID NO:155); a CDR-L3 comprising the sequence QSYDSSLSGSHVV (SEQ ID NO:156); a CDR-H1 comprising the sequence GGTFSNY (SEQ ID NO:151); a CDR-H2 comprising the sequence IPIFGI (SEQ ID NO:152); and a CDR-H3 comprising the sequence GWWFGELETYYFDY (SEQ ID NO:153);
(t) a CDR-L1 comprising the sequence PGDKLGDKFAC (SEQ ID NO:160); a CDR-L2 comprising the sequence QDNKRPS (SEQ ID NO:161); a CDR-L3 comprising the sequence QAWHSSTVV (SEQ ID NO:162); a CDR-H1 comprising the sequence GYSLNSGY (SEQ ID NO:157); a CDR-H2 comprising the sequence YHSGS (SEQ ID NO:158); and a CDR-H3 comprising the sequence KLVPTAPFDY (SEQ ID NO:159);
(u) a CDR-L1 comprising the sequence SGTSSDVGRYNYVS (SEQ ID NO:165); a CDR-L2 comprising the sequence DVSDRPS (SEQ ID NO:166); a CDR-L3 comprising the sequence TSHTSSTISYVV (SEQ ID NO:167); a CDR-H1 comprising the sequence GFTFSTY (SEQ ID NO:163); a CDR-H2 comprising the sequence SYDGSN (SEQ ID NO:104); and a CDR-H3 comprising the sequence DPHIVVVPAAMRFEA (SEQ ID NO:164);
(v) a CDR-L1 comprising the sequence RSSQSLLHSNGYNYLD (SEQ ID NO:170); a CDR-L2 comprising the sequence LGSNRAS (SEQ ID NO:171); a CDR-L3 comprising the sequence MQALQTPFT (SEQ ID NO:171); a CDR-H1 comprising the sequence GGSISSY (SEQ ID NO:168); a CDR-H2 comprising the sequence YYSGS (SEQ ID NO:96); and a CDR-H3 comprising the sequence APGATYSSGWYYYYYYMDV (SEQ ID NO:169);
(w) a CDR-L1 comprising the sequence RSSQSLLHINGYNYLD (SEQ ID NO:176); a CDR-L2 comprising the sequence LGSNRAS (SEQ ID NO:171); a CDR-L3 comprising the sequence MQALQTPWT (SEQ ID NO:177); a CDR-H1 comprising the sequence GFPFRNY (SEQ ID NO:173); a CDR-H2 comprising the sequence SSRGDT (SEQ ID NO:174); and a CDR-H3 comprising the sequence VQSGFSYGYGFDY (SEQ ID NO:175);
(x) a CDR-L1 comprising the sequence TGTSSDVGSYNLVS (SEQ ID NO:179); a CDR-L2 comprising the sequence EVSKRPS (SEQ ID NO:93); a CDR-L3 comprising the sequence CSYAGSSTSYVV (SEQ ID NO:180); a CDR-H1 comprising the sequence GFTFSSY (SEQ ID NO:89); a CDR-H2 comprising the sequence SGSGGS (SEQ ID NO:90); and a CDR-H3 comprising the sequence DGAVATGPGYFYFYMDV (SEQ ID NO:178);
(y) a CDR-L1 comprising the sequence RASQGISSALA (SEQ ID NO:182); a CDR-L2 comprising the sequence DASSLES (SEQ ID NO:107); a CDR-L3 comprising the sequence QQFNNYPLT (SEQ ID NO:108); a CDR-H1 comprising the sequence GFTFSSY (SEQ ID NO:89); a CDR-H2 comprising the sequence SYDGSN (SEQ ID NO:104); and a CDR-H3 comprising the sequence DLEYYGSGSYSLFDY (SEQ ID NO:181);
(z) a CDR-L1 comprising the sequence KSSQSVLYSSNNKNYLA (SEQ ID NO:98); a CDR-L2 comprising the sequence WASTRES (SEQ ID NO:99); a CDR-L3 comprising the sequence QQYYSTPLT (SEQ ID NO:231); a CDR-H1 comprising the sequence GFTFSSY (SEQ ID NO:89); a CDR-H2 comprising the sequence SYDGSN (SEQ ID NO:104); and a CDR-H3 comprising the sequence GGGGYNTFFDY (SEQ ID NO:183);
(aa) a CDR-L1 comprising the sequence QASQDISNFLN (SEQ ID NO:186); a CDR-L2 comprising the sequence DASNLET (SEQ ID NO:118); a CDR-L3 comprising the sequence QHYTT (SEQ ID NO:187); a CDR-H1 comprising the sequence GGSIRSYSH (SEQ ID NO:184); a CDR-H2 comprising the sequence YYSGS (SEQ ID NO:96); and a CDR-H3 comprising the sequence TIPTYDDILTGYQFDY (SEQ ID NO:185);
(bb) a CDR-L1 comprising the sequence SGDKLGDKYVC (SEQ ID NO:190); a CDR-L2 comprising the sequence QDTKRPS (SEQ ID NO:191); a CDR-L3 comprising the sequence QAWDSSTGV (SEQ ID NO:192); a CDR-H1 comprising the sequence GFTFSSY (SEQ ID NO:89); a CDR-H2 comprising the sequence KQDGSE (SEQ ID NO:188); and a CDR-H3 comprising the sequence VGLSSWYFEY (SEQ ID NO:189);
(cc) a CDR-L1 comprising the sequence TGSSSDVGGYDFVS; a CDR-L2 comprising the sequence DVTNRPS (SEQ ID NO:197); a CDR-L3 comprising the sequence SSYTSSSTRV (SEQ ID NO:198); a CDR-H1 comprising the sequence GYIFTDY (SEQ ID NO:193); a CDR-H2 comprising the sequence NPNSGG (SEQ ID NO:194); and a CDR-H3 comprising the sequence EASLNRSRYYSSGGTVYYYYYYMDV (SEQ ID NO:195);
(dd) a CDR-L1 comprising the sequence RASQGIRNDLG (SEQ ID NO:232); a CDR-L2 comprising the sequence AASSLQS (SEQ ID NO:207); a CDR-L3 comprising the sequence LQYNSYPRT (SEQ ID NO:233); a CDR-H1 comprising the sequence GYSISSGY (SEQ ID NO:199); a CDR-H2 comprising the sequence YHSGS (SEQ ID NO:158); and a CDR-H3 comprising the sequence DHGSYDFWSGYSRDAFDI (SEQ ID NO:200);
(ee) a CDR-L1 comprising the sequence RASQSISSWLA (SEQ ID NO:234); a CDR-L2 comprising the sequence KASSLES (SEQ ID NO:235); a CDR-L3 comprising the sequence QQYNTYSFT (SEQ ID NO:236); a CDR-H1 comprising the sequence GFSVSSN (SEQ ID NO:201); a CDR-H2 comprising the sequence YSGGS (SEQ ID NO:202); and a CDR-H3 comprising the sequence GYGDSQR (SEQ ID NO:203); or
(ff) a CDR-L1 comprising the sequence RASQSINNYLN (SEQ ID NO:206); a CDR-L2 comprising the sequence AASSLQS (SEQ ID NO:207); a CDR-L3 comprising the sequence QQSYSPYT (SEQ ID NO:208); a CDR-H1 comprising the sequence GGSVSSDNY (SEQ ID NO:204); a CDR-H2 comprising the sequence YYSGS (SEQ ID NO:96); and a CDR-H3 comprising the sequence GFVATYYYYMDV (SEQ ID NO:205).

2. The antibody or antigen binding fragment thereof according to item 1, which comprises the following combinations of CDRs: a CDR-L1 comprising the sequence RASQGISSWLA (SEQ ID NO:212); a CDR-L2 comprising the sequence AASSLQS (SEQ ID NO:207); a CDR-L3 comprising the sequence QQGNSFPYT (SEQ ID NO:213); a CDR-H1 comprising the sequence GYTFTRY (SEQ ID NO:209); a CDR-H2 comprising the sequence YPGDSD (SEQ ID NO:210); and a CDR-H3 comprising the sequence LPQYCSNGVCQRWFDP (SEQ ID NO:211).

3. The antibody or an antigen binding fragment thereof according to item 1, which comprises the following combinations of CDRs: a CDR-L1 comprising the sequence RASQSVSSSYLA (SEQ ID NO:80); a CDR-L2 comprising the sequence GASSRAT (SEQ ID NO:81); a CDR-L3 comprising the sequence QQYGTSPWT (SEQ ID NO:82); a CDR-H1 comprising the sequence GFTFTSS (SEQ ID NO:77); a CDR-H2 comprising the sequence VVGSGN (SEQ ID NO:78); and a CDR-H3 comprising the sequence PSCSGGRCYDGFDI (SEQ ID NO:79).

4. The antibody or an antigen binding fragment thereof according to item 1, which comprises the following combinations of heavy chain and light chain variable regions:
(i) a heavy chain variable region comprising an amino acid sequence having at least 70% identity with the sequence of SEQ ID NO: 35; and a light chain variable region comprising an amino acid sequence having at least 70% identity with the sequence of SEQ ID NO: 36;
(ii) a heavy chain variable region comprising an amino acid sequence having at least 70% identity with the sequence of SEQ ID NO: 1; and a light chain variable region comprising an amino acid sequence having at least 70% identity with the sequence of SEQ ID NO: 2;
(iii) a heavy chain variable region comprising an amino acid sequence having at least 70% identity with the sequence of SEQ ID NO: 3; and a light chain variable region comprising an amino acid sequence having at least 70% identity with the sequence of SEQ ID NO: 4;
(iv) a heavy chain variable region comprising an amino acid sequence having at least 70% identity with the sequence of SEQ ID NO: 5; and a light chain variable region comprising an amino acid sequence having at least 70% identity with the sequence of SEQ ID NO: 6;
(v) a heavy chain variable region comprising an amino acid sequence having at least 70% identity with the sequence of SEQ ID NO: 7; and a light chain variable region comprising an amino acid sequence having at least 70% identity with the sequence of SEQ ID NO: 8;
(vi) a heavy chain variable region comprising an amino acid sequence having at least 70% identity with the sequence of SEQ ID NO: 9; and a light chain variable region comprising an amino acid sequence having at least 70% identity with the sequence of SEQ ID NO: 10;
(vii) a heavy chain variable region comprising an amino acid sequence having at least 70% identity with the sequence of SEQ ID NO: 11; and a light chain variable region comprising an amino acid sequence having at least 70% identity with the sequence of SEQ ID NO: 12;
(viii) a heavy chain variable region comprising an amino acid sequence having at least 70% identity with the sequence of SEQ ID NO: 13; and a light chain variable region comprising an amino acid sequence having at least 70% identity with the sequence of SEQ ID NO: 14;
(ix) a heavy chain variable region comprising an amino acid sequence having at least 70% identity with the sequence of SEQ ID NO: 15; and a light chain variable region comprising an amino acid sequence having at least 70% identity with the sequence of SEQ ID NO: 16;
(x) a heavy chain variable region comprising an amino acid sequence having at least 70% identity with the sequence of SEQ ID NO: 17; and a light chain variable region comprising an amino acid sequence having at least 70% identity with the sequence of SEQ ID NO: 18;
(xi) a heavy chain variable region comprising an amino acid sequence having at least 70% identity with the sequence of SEQ ID NO: 19; and a light chain variable region comprising an amino acid sequence having at least 70% identity with the sequence of SEQ ID NO: 20;
(xii) a heavy chain variable region comprising an amino acid sequence having at least 70% identity with the sequence of SEQ ID NO: 21; and a light chain variable region comprising an amino acid sequence having at least 70% identity with the sequence of SEQ ID NO: 22;
(xiii) a heavy chain variable region comprising an amino acid sequence having at least 70% identity with the sequence of SEQ ID NO: 23; and a light chain variable region comprising an amino acid sequence having at least 70% identity with the sequence of SEQ ID NO: 24;
(xiv) a heavy chain variable region comprising an amino acid sequence having at least 70% identity with the sequence of SEQ ID NO: 25; and a light chain variable region comprising an amino acid sequence having at least 70% identity with the sequence of SEQ ID NO: 26;
(xv) a heavy chain variable region comprising an amino acid sequence having at least 70% identity with the sequence of SEQ ID NO: 27; and a light chain variable region comprising an amino acid sequence having at least 70% identity with the sequence of SEQ ID NO: 28;
(xvi) a heavy chain variable region comprising an amino acid sequence having at least 70% identity with the sequence of SEQ ID NO: 29; and a light chain variable region comprising an amino acid sequence having at least 70% identity with the sequence of SEQ ID NO: 30;
(xvii) a heavy chain variable region comprising an amino acid sequence having at least 70% identity with the sequence of SEQ ID NO: 31; and a light chain variable region comprising an amino acid sequence having at least 70% identity with the sequence of SEQ ID NO: 32;
(xviii) a heavy chain variable region comprising an amino acid sequence having at least 70% identity with the sequence of SEQ ID NO: 33; and a light chain variable region comprising an amino acid sequence having at least 70% identity with the sequence of SEQ ID NO: 34;
(xix) a heavy chain variable region comprising an amino acid sequence having at least 70% identity with the sequence of SEQ ID NO: 37; and a light chain variable region comprising an amino acid sequence having at least 70% identity with the sequence of SEQ ID NO: 38;
(xx) a heavy chain variable region comprising an amino acid sequence having at least 70% identity with the sequence of SEQ ID NO: 39; and a light chain variable region comprising an amino acid sequence having at least 70% identity with the sequence of SEQ ID NO: 40;
(xxi) a heavy chain variable region comprising an amino acid sequence having at least 70% identity with the sequence of SEQ ID NO: 41; and a light chain variable region comprising an amino acid sequence having at least 70% identity with the sequence of SEQ ID NO: 42;
(xxii) a heavy chain variable region comprising an amino acid sequence having at least 70% identity with the sequence of SEQ ID NO: 43; and a light chain variable region comprising an amino acid sequence having at least 70% identity with the sequence of SEQ ID NO: 44;
(xxiii) a heavy chain variable region comprising an amino acid sequence having at least 70% identity with the sequence of SEQ ID NO: 45; and a light chain variable region comprising an amino acid sequence having at least 70% identity with the sequence of SEQ ID NO: 46;
(xxiv) a heavy chain variable region comprising an amino acid sequence having at least 70% identity with the sequence of SEQ ID NO: 47; and a light chain variable region comprising an amino acid sequence having at least 70% identity with the sequence of SEQ ID NO: 48;
(xxv) a heavy chain variable region comprising an amino acid sequence having at least 70% identity with the sequence of SEQ ID NO: 49; and a light chain variable region comprising an amino acid sequence having at least 70% identity with the sequence of SEQ ID NO: 50;
(xxvi) a heavy chain variable region comprising an amino acid sequence having at least 70% identity with the sequence of SEQ ID NO: 51; and a light chain variable region comprising an amino acid sequence having at least 70% identity with the sequence of SEQ ID NO: 52;
(xxvii) a heavy chain variable region comprising an amino acid sequence having at least 70% identity with the sequence of SEQ ID NO: 53; and a light chain variable region comprising an amino acid sequence having at least 70% identity with the sequence of SEQ ID NO: 54;
(xxviii) a heavy chain variable region comprising an amino acid sequence having at least 70% identity with the sequence of SEQ ID NO: 55; and a light chain variable region comprising an amino acid sequence having at least 70% identity with the sequence of SEQ ID NO: 56;
(xxix) a heavy chain variable region comprising an amino acid sequence having at least 70% identity with the sequence of SEQ ID NO: 57; and a light chain variable region comprising an amino acid sequence having at least 70% identity with the sequence of SEQ ID NO: 58;
(xxx) a heavy chain variable region comprising an amino acid sequence having at least 70% identity with the sequence of SEQ ID NO: 59; and a light chain variable region comprising an amino acid sequence having at least 70% identity with the sequence of SEQ ID NO: 60;
(xxxi) a heavy chain variable region comprising an amino acid sequence having at least 70% identity with the sequence of SEQ ID NO: 61; and a light chain variable region comprising an amino acid sequence having at least 70% identity with the sequence of SEQ ID NO: 62; or
(xxxii) a heavy chain variable region comprising an amino acid sequence having at least 70% identity with the sequence of SEQ ID NO: 63; and a light chain variable region comprising an amino acid sequence having at least 70% identity with the sequence of SEQ ID NO: 64.

5. The antibody or antigen binding fragment thereof according to item 4, which comprises the following combination of heavy chain and light chain variable regions: a heavy chain variable region comprising an amino acid sequence having at least 70% identity with the sequence of SEQ ID NO: 35; and a light chain variable region comprising an amino acid sequence having at least 70% identity with the sequence of SEQ ID NO: 36.

6. The antibody or antigen binding fragment thereof according to item 4, which comprises the following combination of heavy chain and light chain variable regions: a heavy chain variable region comprising an amino acid sequence having at least 70% identity with the sequence of SEQ ID NO: 1; and a light chain variable region comprising an amino acid sequence having at least 70% identity with the sequence of SEQ ID NO: 2.

7. The antibody or antigen binding fragment thereof according to any one of items 1 to 6, which is a fully human antibody.

8. The antibody or antigen binding fragment thereof according to any one of items 1 to 7, which is an IgG antibody.

9. The antibody or antigen binding fragment thereof according to any one of items 1 to 8, which is a recombinant antibody or antigen binding fragment thereof.

10. A conjugate comprising the antibody or antigen binding fragment thereof of any one of items 1 to 9.

11. A nucleic acid comprising a sequence encoding the light and heavy chain of the antibody or antigen binding fragment thereof of any one of items 1 to 9; or a first nucleic acid comprising a sequence encoding the light chain of the antibody or antigen binding fragment thereof of any one of items 1 to 9 and a second nucleic acid comprising a sequence encoding the heavy chain of the antibody or antigen binding fragment thereof of any one of items 1 to 9.

12. A host cell comprising the nucleic acid(s) of item 11.

13. A mixture comprising at least two of the antibodies or antigen binding fragments thereof of any one of items 1 to 9.

14. The mixture of item 13, which comprises the antibody or antigen binding fragment thereof of item 2 and the antibody or antigen binding fragment thereof of item 3.

15. The mixture of item 13, which comprises the antibody or antigen binding fragment thereof of item 5 and the antibody or antigen binding fragment thereof of item 6.

16. The mixture of any one of items 13 to 15, wherein the at least two antibodies are fully human antibodies.

17. A pharmaceutical composition comprising the antibody or antigen binding fragment thereof of any one of items 1 to 9, the conjugate of item 10, the nucleic acid(s) of item 11 or the mixture of any one of items 13 to 16, and a pharmaceutically acceptable excipient.

18. The pharmaceutical composition of item 17, wherein the pharmaceutical composition is in the form of an aerosol or an injectable solution.

19. The pharmaceutical composition of item 17, wherein the pharmaceutical composition is formulated for administration by inhalation.

20. The pharmaceutical composition of item 19, wherein the pharmaceutical composition is formulated for administration by a nebulizer.

21. A method for preventing or treating a betacoronavirus infection or a related disease in a subject in need thereof, the method comprising administering to the subject an effective amount of the antibody or antigen binding fragment thereof of any one of items 1 to 9, the nucleic acid(s) of item 11, the mixture of any one of items 13 to 16, or the pharmaceutical composition of any one of items 17 to 20.

22. A method for reducing the risk of developing a betacoronavirus-related disease or the severity of a betacoronavirus-related disease in a subject, the method comprising administering to the subject an effective amount of the antibody or antigen binding fragment thereof of any one of items 1 to 9, the nucleic acid(s) of item 11, the mixture of any one of items 13 to 16, or the pharmaceutical composition of any one of items 17 to 20.

23. A method for blocking the entry of a betacoronavirus in an ACE2-expressing cell, the method comprising contacting the cell and/or the virus with an effective amount of the antibody or antigen binding fragment thereof of any one of items 1 to 9, the nucleic acid(s) of item 11, the mixture of any one of items 13 to 16, or the pharmaceutical composition of any one of items 17 to 20.

24. The method of any one of items 21 to 23, wherein the betacoronavirus is a sarbecovirus. 25. The method of item 24, wherein the sarbecovirus is SARS-CoV-2.

26. The method of item 25, wherein the SARS-CoV-2 is a variant of the Wuhan original SARS-CoV-2 strain.

27. The method of any one of items 21 to 26, wherein the antibody, antigen-binding fragment thereof, nucleic acid, mixture, or pharmaceutical composition is administered with (i) at least one additional anti-SARS-CoV-2 antibody or antigen-binding fragment thereof, or nucleic acid(s) encoding said at least one additional anti-SARS-CoV-2 antibody or antigen-binding fragment thereof; and/or (ii) at least one antiviral or anti-inflammatory drug.

28. The method of any one of items 21 to 27, wherein the subject is an immunosuppressed or immunocompromised subject.

29. Use of the antibody or antigen binding fragment thereof of any one of items 1 to 9, the nucleic acid(s) of item 11, the mixture of any one of items 13 to 16, or the pharmaceutical composition of any one of items 17 to 20 for preventing or treating a betacoronavirus infection or a related disease in a subject.

30. Use of the antibody or antigen binding fragment thereof of any one of items 1 to 9, the nucleic acid(s) of item 11, the mixture of any one of items 13 to 16, or the pharmaceutical composition of any one of items 17 to 20 for the manufacture of a medicament for preventing or treating a betacoronavirus infection or a related disease in a subject.

31. Use of the antibody or antigen binding fragment thereof of any one of items 1 to 9, the nucleic acid(s) of item 11, the mixture of any one of items 13 to 16, or the pharmaceutical composition of any one of items 17 to 20 for reducing the risk of developing a betacoronavirus-related disease or the severity of a betacoronavirus-related disease in a subject.

32. Use of the antibody or antigen binding fragment thereof of any one of items 1 to 9, the nucleic acid(s) of item 11, the mixture of any one of items 13 to 16, or the pharmaceutical composition of any one of items 17 to 20 for the manufacture of a medicament for reducing the risk of developing a betacoronavirus-related disease or the severity of a betacoronavirus-related disease in a subject.

33. Use of the antibody or antigen binding fragment thereof of any one of items 1 to 9, the nucleic acid(s) of item 11, the mixture of any one of items 13 to 16, or the pharmaceutical composition of any one of items 17 to 20 for blocking the entry of a betacoronavirus in an ACE2-expressing cell.

34. Use of the antibody or antigen binding fragment thereof of any one of items 1 to 9, the nucleic acid(s) of item 11, the mixture of any one of items 13 to 16, or the pharmaceutical composition of any one of items 17 to 20 for the manufacture of a medicament for blocking the entry of a betacoronavirus in an ACE2-expressing cell.

35. The use of any one of items 29 to 34, wherein the betacoronavirus is a sarbecovirus.

36. The use of item 35, wherein the sarbecovirus is SARS-CoV-2.

37. The use of item 36, wherein the SARS-CoV-2 is a variant of the Wuhan original SARS-CoV-2 strain.

38. The use of any one of items 29 to 37, wherein the antibody, antigen-binding fragment thereof, nucleic acid, mixture, or pharmaceutical composition is for administration with (i) at least one additional anti-SARS-CoV-2 antibody or antigen-binding fragment thereof, or nucleic acid(s) encoding said at least one additional anti-SARS-CoV-2 antibody or antigen-binding fragment thereof; and/or (ii) at least one antiviral or anti-inflammatory drug.

39. The use of any one of 29 to 38, wherein the subject is an immunosuppressed or immunocompromised subject.

40. The antibody or antigen binding fragment thereof of any one of items 1 to 9, the nucleic acid(s) of item 11, the mixture of any one of items 13 to 16, or the pharmaceutical composition of any one of items 17 to 20 for use in preventing or treating a betacoronavirus infection or a related disease in a subject.

41. The antibody or antigen binding fragment thereof of any one of items 1 to 9, the nucleic acid(s) of item 11, the mixture of any one of items 13 to 16, or the pharmaceutical composition of any one of items 17 to 20 for use in reducing the risk of developing a betacoronavirus-related disease or the severity of a betacoronavirus-related disease in a subject.

42. The antibody or antigen binding fragment thereof of any one of items 1 to 9, the nucleic acid(s) of item 11, the mixture of any one of items 13 to 16, or the pharmaceutical composition of any one of items 17 to 20 for use in blocking the entry of a betacoronavirus in an ACE2-expressing cell.

43. The antibody, antigen-binding fragment thereof, mixture, or pharmaceutical composition for use according to any one of items 40 to 42, wherein the betacoronavirus is a sarbecovirus.

44. The antibody, antigen-binding fragment thereof, mixture, or pharmaceutical composition for use according to item 43, wherein the sarbecovirus is SARS-CoV-2.

45. The antibody, antigen-binding fragment thereof, mixture, or pharmaceutical composition for use according to item 44, wherein the SARS-CoV-2 is a variant of the Wuhan original SARS-CoV-2 strain.

46. The antibody, antigen-binding fragment thereof, mixture, or pharmaceutical composition for use according to any one of items 40 to 45, wherein the antibody, antigen-binding fragment thereof, mixture or cocktail, or pharmaceutical composition is for administration with (i) at least one additional anti-SARS-CoV-2 antibody or antigen-binding fragment thereof, or nucleic acid(s) encoding said at least one additional anti-SARS-CoV-2 antibody or antigen-binding fragment thereof; and/or (ii) at least one antiviral or anti-inflammatory drug.

47. The antibody, antigen-binding fragment thereof, mixture or cocktail, or pharmaceutical composition for use according to any one of 40 to 46, wherein the subject is an immunosuppressed or immunocompromised subject.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows the purification of S2P and RBD from SARS-CoV-2. Size exclusion chromatography traces of S2P and RBD. SDS-page under non-reducing (lines 1 and 3) and reducing (lines 2 and 4) conditions of S2P (lines 1-2) and RBD (lines 3-4).

FIG. 2 shows the serum antibody reactivity to S2P and RBD from SARS-CoV-2. COVID19(+): sera from three confirmed patients infected with SARS-CoV-2 E_COV_1-8, 10: Nine sera from persons infected with endemic CoV viruses. Negative Control: two sera from healthy individuals. The graphs represent total antibody response to S2P and RBD (IgG, IgM and IgA);

FIGS. 3A-B show the results of SARS CoV-2 pseudovirus neutralization assay. FIG. 3A: Entry SARS CoV-2 S pseudotyped HIV-1 into 293T cells expressing ACE2 are inhibited by anti-ACE2 antibodies but not by an anti-EBV mAB (AMM01). FIG. 3B: Plasma from COVID-19 positive donors. but not negative control plasma collected from a healthy donor prior to the pandemic neutralize SARS CoV-2 pseudovirus infection of 293 cells expressing ACE2;

FIG. 4 shows a B cell-staining with S2P and RBD. Class switched (IgM− IgG+) B cells were stained with S2P labeled with BV710 or PE and with RBD labeled with APC. Single. BV710+/PE+ B cells (left panel) were sorted into individual wells of a 96 well plate. Sorted cells were monitored for RBD binding using indexed sorting (right panel);

FIGS. 5A-5E show the binding to S2P and RBD of several mAbs isolated from COVID-19 infected patients using the strategy depicted in FIG. 4. COVID-19-derived mAbs were loaded onto an anti-human Fc probe and dipped in the SARS-CoV2 recombinant envelope proteins to measure binding using biolayer interferometry (BLI);

FIG. 6 shows the neutralizing activity of selected antibodies (Mab #1, Mab #7, Mab #25 and Mab #43) measured in a SARS-CoV-2 pseudovirus assay. Dose-response curves are shown on the left and the interpolated concentration that reduces infectivity by half (IC₅₀) is shown on the right;

FIGS. 7A-7K show the amino acid sequences of the heavy and light (kappa) chain variable regions of the antibodies isolated from SARS-CoV2-infected patients described herein. The amino acid corresponding to the CDR1, CDR2 and CDR3 according to the Chothia numbering scheme are underlined;

FIG. 8A is a graph showing the ability of Mab #25 to neutralize the indicated SARS-CoV-2 variants of concern and the SARS-like bat virus WIV1.

FIG. 8B is a graph showing the ability of an RBD-directed mAb (Mab #30) to neutralize the indicated SARS-CoV-2 variants of concern and the SARS-like bat virus WIV1.

FIG. 8C shows the neutralizing activity of Mab #1 against the Alpha, Beta, Gamma, Delta and Omicron SARS-CoV-2 variants of concern measured in a SARS-CoV-2 pseudovirus assay. Dose-response curves are shown on the graph (top) and the interpolated concentration that reduces infectivity by half (IC₅₀) is shown under the graph;

FIG. 8D shows the neutralizing activity of Mab #25 against the Alpha, Beta, Gamma, Delta and Omicron SARS-CoV-2 variants of concern measured in a SARS-CoV-2 pseudovirus assay. Dose-response curves are shown on the graph (top) and the interpolated concentration that reduces infectivity by half (IC₅₀) is shown under the graph;

FIG. 8E shows biolayer interferometry (BLI) traces of Mab #25 incubated with human coronavirus antigens as indicated. BLI experiments were performed on an Octet Red instrument at 30° C. with shaking at 500-1000 rpm. All loading steps were 300 s, followed by a 60 s baseline in KB buffer (1×PBS, 0.01% Tween™ 20, 001% BSA, and 0.005% NaN₃, pH 7.4), and then a 300 s association phase and a 300 s dissociation phase in KB. For the binding BLI experiments, mAbs were loaded at a concentration of 20 mg/mL in PBS onto Anti-Human IgG Fc capture (AHC) biosensors. After baseline, probes were dipped in either SARS-CoV2 proteins; SARS-CoV-2 RBD, S-2P, S1, S1 NTD or S2; SARS-CoV proteins; SARS-CoV-RBD or S-2P, or human coronavirus spike proteins; HCoV2-OC43, HKU1, NL63 or 229, at a concentration of 2-0.5 mM for the association phase. The binding of mature VRC01 was used as negative control to subtract the baseline binding in all of these experiments.

FIG. 8F is a graph showing the binding of Mab #25 to 15mer peptides that overlap by 11 amino acids spanning residues 1133-1171 of the SARS-CoV-2 spike protein, and to a 15mer peptide derived from an HIV-1 Env protein, as measured by ELISA. MaxiSorp® microtiter plates (Thermo Scientific Cat #464718) were coated with 300 ng/well of streptavidin (New England Biolabs Catalog #: N7021S) overnight at room temperature. Plates were washed 4× with PBS with 0.02% Tween™-20 (wash buffer), then incubated with 60 μL/well of 3% BSA and 0.02% Tween™-20 in PBS (blocking buffer) for 1 hr at 37° C. After washing 4× with wash buffer, 380 ng/well of biotinylated peptides diluted in blocking buffer were incubated for 1 hr at 37° C. Plates were washed 4× in wash buffer and then Mab #25 was serially diluted in blocking buffer, added to the plate and incubated for 1 hr at 37° C. Plates were washed 4× in wash buffer and the secondary antibody Goat anti-Human Ig-HRP (Southern Biotech, Cat #2010-05), was added and incubated at 37° C. for 1 hr. Plates were washed 4× wash buffer, and then 30 μL/well of SureBlue® Reserve TMB Peroxidase Substrate (Seracare KPL, Cat #5120-0080) was added and incubated for 3 min followed by addition of 30 μL of 1 N H₂SO₄ to stop the reaction. The optical density at 450 nm was measured using a SpectraMax® i3× plate reader (Molecular Devices). All wash steps were performed using a BioTek 405/TS Microplate Washer.

FIG. 8G is an alanine scanning plot of the stem helix region that Mab #25 binds. Mab #25 binding to linear peptides corresponding to amino acids 1153-1167 of the SARS-CoV-2 spike, where each amino acid was substituted by alanine was measured by ELISA. The absorbance at 450 nm resulting from the addition of 1.25 μg of Mab #25 is shown. Each dot represents a technical replicate from three independent experiments conducted in duplicate.

FIG. 8H is a graph showing the binding of Mab #25 to linear stem helix peptides from diverse beta coronaviruses (SARS-CoV-1/2/WIV1, MERS-CoV, HCoV-OC43, and HCoV-HKU1) measured by ELISA.

### DETAILED DISCLOSURE

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

The use of any and all examples, or exemplary language (e.g., “such as”) provided herein, is intended merely to better illustrate the invention and does not pose a limitation on the scope of the invention unless otherwise claimed.

No language in the specification should be construed as indicating any non-claimed element as essential to the practice of the invention.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

As described herein severe SARS-CoV-2 infection refers to individuals infected with SARS-CoV-2 that develop difficulty breathing or persistent chest pressure or pain. Severe SARS-CoV-2 infection may require hospitalization, supplemental oxygen, and or mechanical ventilation. Many individuals are at high risk for severe SARS-CoV-2 including the elderly, diabetic, or those with pre-existing cardiovascular disease.

As described herein acute respiratory distress (ARDs) refers to the fluid build-up of lung alveoli as a result of trauma or infection. ARDs is a significant life-threatening complication of many viral infections including SARS-CoV-2. The antibodies and methods described herein can prevent or improve the prognosis of an individual suffering from SARS-CoV-2 related ARDs.

As used herein, the term “subject” is taken to mean warm blooded animals such as mammals, for example, cats, dogs, mice, guinea pigs, horses, bovine cows, sheep and humans. In an embodiment, the subject is a mammal, and more particularly a human.

Unless otherwise defined, all technical and scientific terms used herein have the same meaning as commonly understood by one of ordinary skill in the art to which this invention belongs.

Disclosed herein are antibodies and antigen-binding fragments thereof that bind to, e.g., neutralize, the severe acute respiratory syndrome coronavirus 2 (SARS-CoV-2).

The present disclosure provides an antibody or an antigen binding fragment thereof comprising one of the following combinations of complementarity determining regions (CDRs):
(a) a light chain CDR1 (CDR-L1) comprising or consisting of an amino acid sequence having at least 70%, 75%, 80%, 85%, 90%, 95% or 100% identity with the sequence RASQSVSSSYLA (SEQ ID NO:80); a CDR-L2 comprising or consisting of an amino acid sequence having at least 70%, 75%, 80%, 85%, 90%, 95% or 100% identity with the sequence GASSRAT (SEQ ID NO:81); a CDR-L3 comprising or consisting of an amino acid sequence having at least 70%, 75%, 80%, 85%, 90%, 95% or 100% identity with the sequence QQYGTSPWT (SEQ ID NO:82); a heavy chain CDR1 (CDR-H1) comprising or consisting of an amino acid sequence having at least 70%, 75%, 80%, 85%, 90%, 95% or 100% identity with the sequence GFTFTSS (SEQ ID NO:77); a CDR-H2 comprising or consisting of an amino acid sequence having at least 70%, 75%, 80%, 85%, 90%, 95% or 100% identity with the sequence VVGSGN (SEQ ID NO:78); and a CDR-H3 comprising or consisting of an amino acid sequence having at least 70%, 75%, 80%, 85%, 90%, 95% or 100% identity with the sequence PSCSGGRCYDGFDI (SEQ ID NO:79);
(b) a CDR-L1 comprising or consisting of an amino acid sequence having at least 70%, 75%, 80%, 85%, 90%, 95% or 100% identity with the sequence RASQTISSWLA (SEQ ID NO:86); a CDR-L2 comprising or consisting of an amino acid sequence having at least 70%, 75%, 80%, 85% or 90% identity with the sequence KASTLES (SEQ ID NO:87); a CDR-L3 comprising or consisting of an amino acid sequence having at least 70%, 75%, 80%, 85%, 90%, 95% or 100% identity with the sequence QQYNSYPWT (SEQ ID NO:88); a CDR-H1 comprising or consisting of an amino acid sequence having at least 70%, 75%, 80%, 85%, 90%, 95% or 100% identity with the sequence GFTFSDY (SEQ ID NO:83); a CDR-H2 comprising or consisting of an amino acid sequence having at least 70%, 75%, 80%, 85%, 90%, 95% or 100% identity with the sequence GSSGSS (SEQ ID NO:84); and a CDR-H3 comprising or consisting of an amino acid sequence having at least 70%, 75%, 80%, 85%, 90%, 95% or 100% identity with the sequence DGSYGDYVRGY (SEQ ID NO:85);
(c) a CDR-L1 comprising or consisting of an amino acid sequence having at least 70%, 75%, 80%, 85%, 90%, 95% or 100% identity with the sequence TGTSSDVGSYNVVS (SEQ ID NO:92); a CDR-L2 comprising or consisting of an amino acid sequence having at least 70%, 75%, 80%, 85%, 90%, 95% or 100% identity with the sequence EVSKRPS (SEQ ID NO:93); a CDR-L3 comprising or consisting of an amino acid sequence having at least 70%, 75%, 80%, 85%, 90%, 95% or 100% identity with the sequence CSYAGSSTSWVV; a CDR-H1 comprising or consisting of an amino acid sequence having at least 70%, 75%, 80%, 85%, 90%, 95% or 100% identity with the sequence GFTFSSY (SEQ ID NO:89); a CDR-H2 comprising or consisting of an amino acid sequence having at least 70%, 75%, 80%, 85%, 90%, 95% or 100% identity with the sequence SGSGGS (SEQ ID NO:90); and a CDR-H3 comprising or consisting of an amino acid sequence having at least 70%, 75%, 80%, 85%, 90%, 95% or 100% identity with the sequence DDSTSAYYYYYYMDV (SEQ ID NO:91);
(d) a CDR-L1 comprising or consisting of an amino acid sequence having at least 70%, 75%, 80%, 85%, 90%, 95% or 100% identity with the sequence KSSQSVLYSSNNKNYLA (SEQ ID NO:98); a CDR-L2 comprising or consisting of an amino acid sequence having at least 70%, 75%, 80%, 85%, 90%, 95% or 100% identity with the sequence WASTRES (SEQ ID NO:99); a CDR-L3 comprising or consisting of an amino acid sequence having at least 70%, 75%, 80%, 85%, 90%, 95% or 100% identity with the sequence QQYYNSYT (SEQ ID NO:100); a CDR-H1 comprising or consisting of an amino acid sequence having at least 70%, 75%, 80%, 85%, 90%, 95% or 100% identity with the sequence GGSISSSSY; a CDR-H2 comprising or consisting of an amino acid sequence having at least 70%, 75%, 80%, 85%, 90%, 95% or 100% identity with the sequence YYSGS (SEQ ID NO:96); and a CDR-H3 comprising or consisting of an amino acid sequence having at least 70%, 75%, 80%, 85%, 90%, 95% or 100% identity with the sequence HPTFSGYEYYFDH (SEQ ID NO:97);
(e) a CDR-L1 comprising or consisting of an amino acid sequence having at least 70%, 75%, 80%, 85% or 90% identity with the sequence RASQSVNNYLA (SEQ ID NO:214); a CDR-L2 comprising or consisting of an amino acid sequence having at least 70%, 75%, 80%, 85%, 90%, 95% or 100% identity with the sequence DASHRAT (SEQ ID NO:215); a CDR-L3 comprising or consisting of an amino acid sequence having at least 70%, 75%, 80%, 85%, 90%, 95% or 100% identity with the sequence QQRSNWPLT (SEQ ID NO:216); a CDR-H1 comprising or consisting of an amino acid sequence having at least 70%, 75%, 80%, 85%, 90%, 95% or 100% identity with the sequence GFTFSSY (SEQ ID NO:89); a CDR-H2 comprising or consisting of an amino acid sequence having at least 70%, 75%, 80%, 85%, 90%, 95% or 100% identity with the sequence NNHGGS (SEQ ID NO:101); and a CDR-H3 comprising or consisting of an amino acid sequence having at least 70%, 75%, 80%, 85%, 90%, 95% or 100% identity with the sequence SDTAMVPYNWFDP (SEQ ID NO:102);
(f) a CDR-L1 comprising or consisting of an amino acid sequence having at least 70%, 75%, 80%, 85%, 90%, 95% or 100% identity with the sequence RASQSVRSNLA (SEQ ID NO:217); a CDR-L2 comprising or consisting of an amino acid sequence having at least 70%, 75%, 80%, 85%, 90%, 95% or 100% identity with the sequence GASTRAT (SEQ ID NO:143); a CDR-L3 comprising or consisting of an amino acid sequence having at least 70%, 75%, 80%, 85%, 90%, 95% or 100% identity with the sequence QQYNYWPPYT (SEQ ID NO:218); a CDR-H1 comprising or consisting of an amino acid sequence having at least 70%, 75%, 80%, 85%, 90%, 95% or 100% identity with the sequence GGSLNNY (SEQ ID NO:219); a CDR-H2 comprising or consisting of an amino acid sequence having at least 70%, 75%, 80%, 85%, 90%, 95% or 100% identity with the sequence NHSGS (SEQ ID NO:220); and a CDR-H3 comprising or consisting of an amino acid sequence having at least 70%, 75%, 80%, 85%, 90%, 95% or 100% identity with the sequence GLFLVYYGSGLGGFDY (SEQ ID NO:221);
(g) a CDR-L1 comprising or consisting of an amino acid sequence having at least 70%, 75%, 80%, 85%, 90%, 95% or 100% identity with the sequence RASQDISSALA (SEQ ID NO:106); a CDR-L2 comprising or consisting of an amino acid sequence having at least 70%, 75%, 80%, 85%, 90%, 95% or 100% identity with the sequence DASSLES (SEQ ID NO:107); a CDR-L3 comprising or consisting of an amino acid sequence having at least 70%, 75%, 80%, 85%, 90%, 95% or 100% identity with the sequence QQFNNYPLT (SEQ ID NO:108); a CDR-H1 comprising or consisting of an amino acid sequence having at least 70%, 75%, 80%, 85% or 90% identity with the sequence GFTFSRY (SEQ ID NO:103); a CDR-H2 comprising or consisting of an amino acid sequence having at least 70%, 75%, 80%, 85%, 90%, 95% or 100% identity with the sequence SYDGSN (SEQ ID NO:104); and a CDR-H3 comprising or consisting of an amino acid sequence having at least 70%, 75%, 80%, 85%, 90%, 95% or 100% identity with the sequence DLEYYTSGSYSLFDY (SEQ ID NO:105);
(h) a CDR-L1 comprising or consisting of an amino acid sequence having at least 70%, 75%, 80%, 85%, 90%, 95% or 100% identity with the sequence RASQSVSSTYLA (SEQ ID NO:111); a CDR-L2 comprising or consisting of an amino acid sequence having at least 70%, 75%, 80%, 85%, 90%, 95% or 100% identity with the sequence GASNRAT (SEQ ID NO:112); a CDR-L3 comprising or consisting of an amino acid sequence having at least 70%, 75%, 80%, 85%, 90%, 95% or 100% identity with the sequence QQYGSSPPLT (SEQ ID NO:113); a CDR-H1 comprising or consisting of an amino acid sequence having at least 70%, 75%, 80%, 85%, 90%, 95% or 100% identity with the sequence GFTFSIY (SEQ ID NO:109); a CDR-H2 comprising or consisting of an amino acid sequence having at least 70%, 75%, 80%, 85%, 90%, 95% or 100% identity with the sequence SYDGSN (SEQ ID NO:104); and a CDR-H3 comprising or consisting of an amino acid sequence having at least 70%, 75%, 80%, 85%, 90%, 95% or 100% identity with the sequence GPTYSYMDV (SEQ ID NO:110);
(i) a CDR-L1 comprising or consisting of an amino acid sequence having at least 70%, 75%, 80%, 85%, 90%, 95% or 100% identity with the sequence QASQDISNYLN (SEQ ID NO:117); a CDR-L2 comprising or consisting of an amino acid sequence having at least 70%, 75%, 80%, 85%, 90%, 95% or 100% identity with the sequence DASNLET (SEQ ID NO:118); a CDR-L3 comprising or consisting of an amino acid sequence having at least 70%, 75%, 80%, 85%, 90%, 95% or 100% identity with the sequence QQYNNLPLT (SEQ ID NO:119); a CDR-H1 comprising or consisting of an amino acid sequence having at least 70%, 75%, 80%, 85%, 90%, 95% or 100% identity with the sequence GFTFSYY (SEQ ID NO:114); a CDR-H2 comprising or consisting of an amino acid sequence having at least 70%, 75%, 80%, 85%, 90%, 95% or 100% identity with the sequence YGSGSN (SEQ ID NO:115); and a CDR-H3 comprising or consisting of an amino acid sequence having at least 70%, 75%, 80%, 85%, 90%, 95% or 100% identity with the sequence DQRNAYDSFDF (SEQ ID NO:116);
(j) a CDR-L1 comprising or consisting of an amino acid sequence having at least 70%, 75%, 80%, 85% or 90% identity with the sequence SGSSSNIGNNYVS (SEQ ID NO:123); a CDR-L2 comprising or consisting of an amino acid sequence having at least 70%, 75%, 80%, 85%, 90%, 95% or 100% identity with the sequence DNNKRPS (SEQ ID NO:124); a CDR-L3 comprising or consisting of an amino acid sequence having at least 70%, 75%, 80%, 85%, 90%, 95% or 100% identity with the sequence GTWDSSLSVVL (SEQ ID NO:125); a CDR-H1 comprising or consisting of an amino acid sequence having at least 70%, 75%, 80%, 85%, 90%, 95% or 100% identity with the sequence GFTFGDY (SEQ ID NO:120); a CDR-H2 comprising or consisting of an amino acid sequence having at least 70%, 75%, 80%, 85%, 90%, 95% or 100% identity with the sequence RSKAYGGT (SEQ ID NO:121); and a CDR-H3 comprising or consisting of an amino acid sequence having at least 70%, 75%, 80%, 85%, 90%, 95% or 100% identity with the sequence DLDYYDSSGYYPTYIDY (SEQ ID NO:122);
(k) a CDR-L1 comprising or consisting of an amino acid sequence having at least 70%, 75%, 80%, 85%, 90%, 95% or 100% identity with the sequence TGSGSNIGAGYDVH (SEQ ID NO:222); a CDR-L2 comprising or consisting of an amino acid sequence having at least 70%, 75%, 80%, 85%, 90%, 95% or 100% identity with the sequence GNNNRPS (SEQ ID NO:223); a CDR-L3 comprising or consisting of an amino acid sequence having at least 70%, 75%, 80%, 85%, 90%, 95% or 100% identity with the sequence QSYDSSLSGPVV (SEQ ID NO:224); a CDR-H1 comprising or consisting of an amino acid sequence having at least 70%, 75%, 80%, 85%, 90%, 95% or 100% identity with the sequence GGSISSGNY (SEQ ID NO:126); a CDR-H2 comprising or consisting of an amino acid sequence having at least 70%, 75%, 80%, 85%, 90%, 95% or 100% identity with the sequence YTSGS (SEQ ID NO:127); and a CDR-H3 comprising or consisting of an amino acid sequence having at least 70%, 75%, 80%, 85%, 90%, 95% or 100% identity with the sequence DAYYDFLSGYIPTYNWFDP (SEQ ID NO:225);
(l) a CDR-L1 comprising or consisting of an amino acid sequence having at least 70%, 75%, 80%, 85%, 90%, 95% or 100% identity with the sequence QASQDISNYLN (SEQ ID NO:117); a CDR-L2 comprising or consisting of an amino acid sequence having at least 70%, 75%, 80%, 85%, 90%, 95% or 100% identity with the sequence VASNLET (SEQ ID NO:226); a CDR-L3 comprising or consisting of an amino acid sequence having at least 70%, 75%, 80%, 85%, 90%, 95% or 100% identity with the sequence QQFDNLPYT (SEQ ID NO:227); a CDR-H1 comprising or consisting of an amino acid sequence having at least 70%, 75%, 80%, 85%, 90%, 95% or 100% identity with the sequence GGSISSGTY (SEQ ID NO:228); a CDR-H2 comprising or consisting of an amino acid sequence having at least 70%, 75%, 80%, 85%, 90%, 95% or 100% identity with the sequence YTSGS (SEQ ID NO:127); and a CDR-H3 comprising or consisting of an amino acid sequence having at least 70%, 75%, 80%, 85%, 90%, 95% or 100% identity with the sequence EYSSSYYYFYYMDV (SEQ ID NO:128);
(m) a CDR-L1 comprising or consisting of an amino acid sequence having at least 70%, 75%, 80%, 85%, 90%, 95% or 100% identity with the sequence QASQDISKYLN (SEQ ID NO:132); a CDR-L2 comprising or consisting of an amino acid sequence having at least 70%, 75%, 80%, 85%, 90%, 95% or 100% identity with the sequence DASNLET (SEQ ID NO:118); a CDR-L3 comprising or consisting of an amino acid sequence having at least 70%, 75%, 80%, 85%, 90%, 95% or 100% identity with the sequence QQYDNLPTT (SEQ ID NO:133); a CDR-H1 comprising or consisting of an amino acid sequence having at least 70%, 75%, 80%, 85%, 90%, 95% or 100% identity with the sequence GFTFSNY (SEQ ID NO:129); a CDR-H2 comprising or consisting of an amino acid sequence having at least 70%, 75%, 80%, 85%, 90%, 95% or 100% identity with the sequence LYDGSN (SEQ ID NO:130); and a CDR-H3 comprising or consisting of an amino acid sequence having at least 70%, 75%, 80%, 85%, 90%, 95% or 100% identity with the sequence GGGPYCGGGSCWAHYFDY (SEQ ID NO:131);
(n) a CDR-L1 comprising or consisting of an amino acid sequence having at least 70%, 75%, 80%, 85%, 90%, 95% or 100% identity with the sequence RASQSVSSIYLA (SEQ ID NO:136); a CDR-L2 comprising or consisting of an amino acid sequence having at least 70%, 75%, 80%, 85%, 90%, 95% or 100% identity with the sequence STSSRAV (SEQ ID NO:137); a CDR-L3 comprising or consisting of an amino acid sequence having at least 70%, 75%, 80%, 85%, 90%, 95% or 100% identity with the sequence HQYGSSPWT (SEQ ID NO:138); a CDR-H1 comprising or consisting of an amino acid sequence having at least 70%, 75%, 80%, 85%, 90%, 95% or 100% identity with the sequence GDSISNY (SEQ ID NO:134); a CDR-H2 comprising or consisting of an amino acid sequence having at least 70%, 75%, 80%, 85%, 90%, 95% or 100% identity with the sequence YYSGS (SEQ ID NO:96); and a CDR-H3 comprising or consisting of an amino acid sequence having at least 70%, 75%, 80%, 85%, 90%, 95% or 100% identity with the sequence DFSL (SEQ ID NO:135);
(o) a CDR-L1 comprising or consisting of an amino acid sequence having at least 70%, 75%, 80%, 85%, 90%, 95% or 100% identity with the sequence RASQSVSSNLA (SEQ ID NO:142); a CDR-L2 comprising or consisting of an amino acid sequence having at least 70%, 75%, 80%, 85%, 90%, 95% or 100% identity with the sequence GASTRAT (SEQ ID NO:143); a CDR-L3 comprising or consisting of an amino acid sequence having at least 70%, 75%, 80%, 85%, 90%, 95% or 100% identity with the sequence QQYYNWPPWT (SEQ ID NO:144); a CDR-H1 comprising or consisting of an amino acid sequence having at least 70%, 75%, 80%, 85%, 90%, 95% or 100% identity with the sequence GFIFSRY (SEQ ID NO:139); a CDR-H2 comprising or consisting of an amino acid sequence having at least 70%, 75%, 80%, 85%, 90%, 95% or 100% identity with the sequence SSSTSF (SEQ ID NO:140); and a CDR-H3 comprising or consisting of an amino acid sequence having at least 70%, 75%, 80%, 85%, 90%, 95% or 100% identity with the sequence WIGGDSSGYYPDAFDI (SEQ ID NO:141);
(p) a CDR-L1 comprising or consisting of an amino acid sequence having at least 70%, 75%, 80%, 85%, 90%, 95% or 100% identity with the sequence RASQSVSSNYLA (SEQ ID NO:229); a CDR-L2 comprising or consisting of an amino acid sequence having at least 70%, 75%, 80%, 85%, 90%, 95% or 100% identity with the sequence GASSRAT (SEQ ID NO:81); a CDR-L3 comprising or consisting of an amino acid sequence having at least 70%, 75%, 80%, 85%, 90%, 95% or 100% identity with the sequence QQYGSSLYT (SEQ ID NO:230); a CDR-H1 comprising or consisting of an amino acid sequence having at least 70%, 75%, 80%, 85%, 90%, 95% or 100% identity with the sequence GDSISSY (SEQ ID NO:145); a CDR-H2 comprising or consisting of an amino acid sequence having at least 70%, 75%, 80%, 85%, 90%, 95% or 100% identity with the sequence YYTGS (SEQ ID NO:146); and a CDR-H3 comprising or consisting of an amino acid sequence having at least 70%, 75%, 80%, 85%, 90%, 95% or 100% identity with the sequence LGYNSGWYGGYFEY (SEQ ID NO:147);
(q) a CDR-L1 comprising or consisting of an amino acid sequence having at least 70%, 75%, 80%, 85%, 90%, 95% or 100% identity with the sequence RASQSVSSNLA (SEQ ID NO:142); a CDR-L2 comprising or consisting of an amino acid sequence having at least 70%, 75%, 80%, 85%, 90%, 95% or 100% identity with the sequence GASTRAT (SEQ ID NO:143); a CDR-L3 comprising or consisting of an amino acid sequence having at least 70%, 75%, 80%, 85%, 90%, 95% or 100% identity with the sequence QQYNKWPPIT (SEQ ID NO:150); a CDR-H1 comprising or consisting of an amino acid sequence having at least 70%, 75%, 80%, 85%, 90%, 95% or 100% identity with the sequence GFTFSSY (SEQ ID NO:89); a CDR-H2 comprising or consisting of an amino acid sequence having at least 70%, 75%, 80%, 85%, 90%, 95% or 100% identity with the sequence SYDGIN (SEQ ID NO:148); and a CDR-H3 comprising or consisting of an amino acid sequence having at least 70%, 75%, 80%, 85%, 90%, 95% or 100% identity with the sequence MYSGSYLGYFDY (SEQ ID NO:149);
(r) a CDR-L1 comprising or consisting of an amino acid sequence having at least 70%, 75%, 80%, 85%, 90%, 95% or 100% identity with the sequence RASQGISSWLA (SEQ ID NO:212); a CDR-L2 comprising or consisting of an amino acid sequence having at least 70%, 75%, 80%, 85%, 90%, 95% or 100% identity with the sequence AASSLQS (SEQ ID NO:207); a CDR-L3 comprising or consisting of an amino acid sequence having at least 70%, 75%, 80%, 85%, 90%, 95% or 100% identity with the sequence QQGNSFPYT (SEQ ID NO:213); a CDR-H1 comprising or consisting of an amino acid sequence having at least 70%, 75%, 80%, 85%, 90%, 95% or 100% identity with the sequence GYTFTRY (SEQ ID NO:209); a CDR-H2 comprising or consisting of an amino acid sequence having at least 70%, 75%, 80%, 85%, 90%, 95% or 100% identity with the sequence YPGDSD (SEQ ID NO:210); and a CDR-H3 comprising or consisting of an amino acid sequence having at least 70%, 75%, 80%, 85%, 90%, 95% or 100% identity with the sequence LPQYCSNGVCQRWFDP (SEQ ID NO:211);
(s) a CDR-L1 comprising or consisting of an amino acid sequence having at least 70%, 75%, 80%, 85%, 90%, 95% or 100% identity with the sequence TGSSSNIGAGYDVH (SEQ ID NO:154); a CDR-L2 comprising or consisting of an amino acid sequence having at least 70%, 75%, 80%, 85%, 90%, 95% or 100% identity with the sequence DNNNRPS (SEQ ID NO:155); a CDR-L3 comprising or consisting of an amino acid sequence having at least 70%, 75%, 80%, 85%, 90%, 95% or 100% identity with the sequence QSYDSSLSGSHVV (SEQ ID NO:156); a CDR-H1 comprising or consisting of an amino acid sequence having at least 70%, 75%, 80%, 85%, 90%, 95% or 100% identity with the sequence GGTFSNY (SEQ ID NO:151); a CDR-H2 comprising or consisting of an amino acid sequence having at least 70%, 75%, 80%, 85%, 90%, 95% or 100% identity with the sequence IPIFGI (SEQ ID NO:152); and a CDR-H3 comprising or consisting of an amino acid sequence having at least 70%, 75%, 80%, 85%, 90%, 95% or 100% identity with the sequence GWWFGELETYYFDY (SEQ ID NO:153);
(t) a CDR-L1 comprising or consisting of an amino acid sequence having at least 70%, 75%, 80%, 85%, 90%, 95% or 100% identity with the sequence PGDKLGDKFAC (SEQ ID NO:160); a CDR-L2 comprising or consisting of an amino acid sequence having at least 70%, 75%, 80%, 85%, 90%, 95% or 100% identity with the sequence QDNKRPS (SEQ ID NO:161); a CDR-L3 comprising or consisting of an amino acid sequence having at least 70%, 75%, 80%, 85%, 90%, 95% or 100% identity with the sequence QAWHSSTVV (SEQ ID NO:162); a CDR-H1 comprising or consisting of an amino acid sequence having at least 70%, 75%, 80%, 85%, 90%, 95% or 100% identity with the sequence GYSLNSGY (SEQ ID NO:157); a CDR-H2 comprising or consisting of an amino acid sequence having at least 70%, 75%, 80%, 85%, 90%, 95% or 100% identity with the sequence YHSGS (SEQ ID NO:158); and a CDR-H3 comprising or consisting of an amino acid sequence having at least 70%, 75%, 80%, 85%, 90%, 95% or 100% identity with the sequence KLVPTAPFDY (SEQ ID NO:159);
(u) a CDR-L1 comprising or consisting of an amino acid sequence having at least 70%, 75%, 80%, 85%, 90%, 95% or 100% identity with the sequence SGTSSDVGRYNYVS (SEQ ID NO:165); a CDR-L2 comprising or consisting of an amino acid sequence having at least 70%, 75%, 80%, 85%, 90%, 95% or 100% identity with the sequence DVSDRPS (SEQ ID NO:166); a CDR-L3 comprising or consisting of an amino acid sequence having at least 70%, 75%, 80%, 85%, 90%, 95% or 100% identity with the sequence TSHTSSTISYVV (SEQ ID NO:167); a CDR-H1 comprising or consisting of an amino acid sequence having at least 70%, 75%, 80%, 85%, 90%, 95% or 100% identity with the sequence GFTFSTY (SEQ ID NO:163); a CDR-H2 comprising or consisting of an amino acid sequence having at least 70%, 75%, 80%, 85%, 90%, 95% or 100% identity with the sequence SYDGSN (SEQ ID NO:104); and a CDR-H3 comprising or consisting of an amino acid sequence having at least 70%, 75%, 80%, 85%, 90%, 95% or 100% identity with the sequence DPHIVVVPAAMRFEA (SEQ ID NO:164);
(v) a CDR-L1 comprising or consisting of an amino acid sequence having at least 70%, 75%, 80%, 85%, 90%, 95% or 100% identity with the sequence RSSQSLLHSNGYNYLD (SEQ ID NO:170); a CDR-L2 comprising or consisting of an amino acid sequence having at least 70%, 75%, 80%, 85%, 90%, 95% or 100% identity with the sequence LGSNRAS (SEQ ID NO:171); a CDR-L3 comprising or consisting of an amino acid sequence having at least 70%, 75%, 80%, 85%, 90%, 95% or 100% identity with the sequence MQALQTPFT (SEQ ID NO:172); a CDR-H1 comprising or consisting of an amino acid sequence having at least 70%, 75%, 80%, 85% or 90% identity with the sequence GGSISSY (SEQ ID NO:168); a CDR-H2 comprising or consisting of an amino acid sequence having at least 70%, 75%, 80%, 85%, 90%, 95% or 100% identity with the sequence YYSGS (SEQ ID NO:96); and a CDR-H3 comprising or consisting of an amino acid sequence having at least 70%, 75%, 80%, 85%, 90%, 95% or 100% identity with the sequence APGATYSSGWYYYYYYMDV (SEQ ID NO:169);
(w) a CDR-L1 comprising or consisting of an amino acid sequence having at least 70%, 75%, 80%, 85%, 90%, 95% or 100% identity with the sequence RSSQSLLHINGYNYLD (SEQ ID NO:176); a CDR-L2 comprising or consisting of an amino acid sequence having at least 70%, 75%, 80%, 85%, 90%, 95% or 100% identity with the sequence LGSNRAS (SEQ ID NO:171); a CDR-L3 comprising or consisting of an amino acid sequence having at least 70%, 75%, 80%, 85%, 90%, 95% or 100% identity with the sequence MQALQTPWT (SEQ ID NO:177); a CDR-H1 comprising or consisting of an amino acid sequence having at least 70%, 75%, 80%, 85%, 90%, 95% or 100% identity with the sequence GFPFRNY (SEQ ID NO:173); a CDR-H2 comprising or consisting of an amino acid sequence having at least 70%, 75%, 80%, 85%, 90%, 95% or 100% identity with the sequence SSRGDT (SEQ ID NO:174); and a CDR-H3 comprising or consisting of an amino acid sequence having at least 70%, 75%, 80%, 85%, 90%, 95% or 100% identity with the sequence VQSGFSYGYGFDY (SEQ ID NO:175);
(x) a CDR-L1 comprising or consisting of an amino acid sequence having at least 70%, 75%, 80%, 85%, 90%, 95% or 100% identity with the sequence TGTSSDVGSYNLVS (SEQ ID NO:179); a CDR-L2 comprising or consisting of an amino acid sequence having at least 70%, 75%, 80%, 85%, 90%, 95% or 100% identity with the sequence EVSKRPS (SEQ ID NO:93); a CDR-L3 comprising or consisting of an amino acid sequence having at least 70%, 75%, 80%, 85%, 90%, 95% or 100% identity with the sequence CSYAGSSTSYVV (SEQ ID NO:180); a CDR-H1 comprising or consisting of an amino acid sequence having at least 70%, 75%, 80%, 85%, 90%, 95% or 100% identity with the sequence GFTFSSY (SEQ ID NO:89); a CDR-H2 comprising or consisting of an amino acid sequence having at least 70%, 75%, 80%, 85%, 90%, 95% or 100% identity with the sequence SGSGGS (SEQ ID NO:90); and a CDR-H3 comprising or consisting of an amino acid sequence having at least 70%, 75%, 80%, 85%, 90%, 95% or 100% identity with the sequence DGAVATGPGYFYFYMDV (SEQ ID NO:178);
(y) a CDR-L1 comprising or consisting of an amino acid sequence having at least 70%, 75%, 80%, 85%, 90%, 95% or 100% identity with the sequence RASQGISSALA (SEQ ID NO:182); a CDR-L2 comprising or consisting of an amino acid sequence having at least 70%, 75%, 80%, 85%, 90%, 95% or 100% identity with the sequence DASSLES (SEQ ID NO:107); a CDR-L3 comprising or consisting of an amino acid sequence having at least 70%, 75%, 80%, 85%, 90%, 95% or 100% identity with the sequence QQFNNYPLT (SEQ ID NO:108); a CDR-H1 comprising or consisting of an amino acid sequence having at least 70%, 75%, 80%, 85%, 90%, 95% or 100% identity with the sequence GFTFSSY (SEQ ID NO:89); a CDR-H2 comprising or consisting of an amino acid sequence having at least 70%, 75%, 80%, 85%, 90%, 95% or 100% identity with the sequence SYDGSN (SEQ ID NO:104); and a CDR-H3 comprising or consisting of an amino acid sequence having at least 70%, 75%, 80%, 85%, 90%, 95% or 100% identity with the sequence DLEYYGSGSYSLFDY (SEQ ID NO:181);
(z) a CDR-L1 comprising or consisting of an amino acid sequence having at least 70%, 75%, 80%, 85%, 90%, 95% or 100% identity with the sequence KSSQSVLYSSNNKNYLA (SEQ ID NO:98); a CDR-L2 comprising or consisting of an amino acid sequence having at least 70%, 75%, 80%, 85%, 90%, 95% or 100% identity with the sequence WASTRES (SEQ ID NO:99); a CDR-L3 comprising or consisting of an amino acid sequence having at least 70%, 75%, 80%, 85%, 90%, 95% or 100% identity with the sequence QQYYSTPLT (SEQ ID NO:231); a CDR-H1 comprising or consisting of an amino acid sequence having at least 70%, 75%, 80%, 85%, 90%, 95% or 100% identity with the sequence GFTFSSY (SEQ ID NO:89); a CDR-H2 comprising or consisting of an amino acid sequence having at least 70%, 75%, 80%, 85%, 90%, 95% or 100% identity with the sequence SYDGSN (SEQ ID NO:104); and a CDR-H3 comprising or consisting of an amino acid sequence having at least 70%, 75%, 80%, 85%, 90%, 95% or 100% identity with the sequence GGGGYNTFFDY (SEQ ID NO:183);
(aa) a CDR-L1 comprising or consisting of an amino acid sequence having at least 70%, 75%, 80%, 85%, 90%, 95% or 100% identity with the sequence QASQDISNFLN (SEQ ID NO:186); a CDR-L2 comprising or consisting of an amino acid sequence having at least 70%, 75%, 80%, 85%, 90%, 95% or 100% identity with the sequence DASNLET (SEQ ID NO:118); a CDR-L3 comprising or consisting of an amino acid sequence having at least 70%, 75%, 80%, 85%, 90%, 95% or 100% identity with the sequence QHYTT (SEQ ID NO:187); a CDR-H1 comprising or consisting of an amino acid sequence having at least 70%, 75%, 80%, 85%, 90%, 95% or 100% identity with the sequence GGSIRSYSH (SEQ ID NO:184); a CDR-H2 comprising or consisting of an amino acid sequence having at least 70%, 75%, 80%, 85%, 90%, 95% or 100% identity with the sequence YYSGS (SEQ ID NO:96); and a CDR-H3 comprising or consisting of an amino acid sequence having at least 70%, 75%, 80%, 85%, 90%, 95% or 100% identity with the sequence TIPTYDDILTGYQFDY (SEQ ID NO:185);
(bb) a CDR-L1 comprising or consisting of an amino acid sequence having at least 70%, 75%, 80%, 85%, 90%, 95% or 100% identity with the sequence SGDKLGDKYVC (SEQ ID NO:190); a CDR-L2 comprising or consisting of an amino acid sequence having at least 70%, 75%, 80%, 85%, 90%, 95% or 100% identity with the sequence QDTKRPS (SEQ ID NO:191); a CDR-L3 comprising or consisting of an amino acid sequence having at least 70%, 75%, 80%, 85%, 90%, 95% or 100% identity with the sequence QAWDSSTGV (SEQ ID NO:192); a CDR-H1 comprising or consisting of an amino acid sequence having at least 70%, 75%, 80%, 85%, 90%, 95% or 100% identity with the sequence GFTFSSY (SEQ ID NO:89); a CDR-H2 comprising or consisting of an amino acid sequence having at least 70%, 75%, 80%, 85%, 90%, 95% or 100% identity with the sequence KQDGSE (SEQ ID NO:188); and a CDR-H3 comprising or consisting of an amino acid sequence having at least 70%, 75%, 80%, 85%, 90%, 95% or 100% identity with the sequence VGLSSWYFEY (SEQ ID NO:189);
(cc) a CDR-L1 comprising or consisting of an amino acid sequence having at least 70%, 75%, 80%, 85%, 90%, 95% or 100% identity with the sequence TGSSSDVGGYDFVS (SEQ ID NO:196); a CDR-L2 comprising or consisting of an amino acid sequence having at least 70%, 75%, 80%, 85%, 90%, 95% or 100% identity with the sequence DVTNRPS (SEQ ID NO:197); a CDR-L3 comprising or consisting of an amino acid sequence having at least 70%, 75%, 80%, 85%, 90%, 95% or 100% identity with the sequence SSYTSSSTRV (SEQ ID NO:198); a CDR-H1 comprising or consisting of an amino acid sequence having at least 70%, 75%, 80%, 85%, 90%, 95% or 100% identity with the sequence GYIFTDY (SEQ ID NO:193); a CDR-H2 comprising or consisting of an amino acid sequence having at least 70%, 75%, 80%, 85%, 90%, 95% or 100% identity with the sequence NPNSGG (SEQ ID NO:194); and a CDR-H3 comprising or consisting of an amino acid sequence having at least 70%, 75%, 80%, 85%, 90%, 95% or 100% identity with the sequence EASLNRSRYYSSGGTVYYYYYYMDV (SEQ ID NO:195);
(dd) a CDR-L1 comprising or consisting of an amino acid sequence having at least 70%, 75%, 80%, 85%, 90%, 95% or 100% identity with the sequence RASQGIRNDLG (SEQ ID NO:232); a CDR-L2 comprising or consisting of an amino acid sequence having at least 70%, 75%, 80%, 85%, 90%, 95% or 100% identity with the sequence AASSLQS (SEQ ID NO:207); a CDR-L3 comprising or consisting of an amino acid sequence having at least 70%, 75%, 80%, 85%, 90%, 95% or 100% identity with the sequence LQYNSYPRT (SEQ ID NO:233); a CDR-H1 comprising or consisting of an amino acid sequence having at least 70%, 75%, 80%, 85%, 90%, 95% or 100% identity with the sequence GYSISSGY (SEQ ID NO:199); a CDR-H2 comprising or consisting of an amino acid sequence having at least 70%, 75%, 80%, 85%, 90%, 95% or 100% identity with the sequence YHSGS (SEQ ID NO:158); and a CDR-H3 comprising or consisting of an amino acid sequence having at least 70%, 75%, 80%, 85%, 90%, 95% or 100% identity with the sequence DHGSYDFWSGYSRDAFDI (SEQ ID NO:200);
(ee) a CDR-L1 comprising or consisting of an amino acid sequence having at least 70%, 75%, 80%, 85%, 90%, 95% or 100% identity with the sequence RASQSISSWLA (SEQ ID NO:234); a CDR-L2 comprising or consisting of an amino acid sequence having at least 70%, 75%, 80%, 85%, 90%, 95% or 100% identity with the sequence KASSLES (SEQ ID NO:235); a CDR-L3 comprising or consisting of an amino acid sequence having at least 70%, 75%, 80%, 85%, 90%, 95% or 100% identity with the sequence QQYNTYSFT (SEQ ID NO:236); a CDR-H1 comprising or consisting of an amino acid sequence having at least 70%, 75%, 80%, 85%, 90%, 95% or 100% identity with the sequence GFSVSSN (SEQ ID NO:201); a CDR-H2 comprising or consisting of an amino acid sequence having at least 70%, 75%, 80%, 85%, 90%, 95% or 100% identity with the sequence YSGGS (SEQ ID NO:202); and a CDR-H3 comprising or consisting of an amino acid sequence having at least 70%, 75%, 80%, 85%, 90%, 95% or 100% identity with the sequence GYGDSQR (SEQ ID NO:203); or
(ff) a CDR-L1 comprising or consisting of an amino acid sequence having at least 70%, 75%, 80%, 85%, 90%, 95% or 100% identity with the sequence RASQSINNYLN (SEQ ID NO:206); a CDR-L2 comprising or consisting of an amino acid sequence having at least 70%, 75%, 80%, 85%, 90%, 95% or 100% identity with the sequence AASSLQS (SEQ ID NO:207); a CDR-L3 comprising or consisting of an amino acid sequence having at least 70%, 75%, 80%, 85%, 90%, 95% or 100% identity with the sequence QQSYSPYT (SEQ ID NO:208); a CDR-H1 comprising or consisting of an amino acid sequence having at least 70%, 75%, 80%, 85%, 90%, 95% or 100% identity with the sequence GGSVSSDNY (SEQ ID NO:204); a CDR-H2 comprising or consisting of an amino acid sequence having at least 70%, 75%, 80%, 85%, 90%, 95% or 100% identity with the sequence YYSGS (SEQ ID NO:96); and a CDR-H3 comprising or consisting of an amino acid sequence having at least 70%, 75%, 80%, 85%, 90%, 95% or 100% identity with the sequence GFVATYYYYMDV (SEQ ID NO:205).

The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules (e.g., single-chain FV, scFV), single domain antibodies (e.g., from camelids), shark NAR single domain antibodies, and multispecific antibodies formed from antibody fragments. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies.

The term “monoclonal antibody” as used herein refers to an antibody from a population of substantially homogeneous antibodies, i.e., the individual antibodies comprising the population are substantially similar and bind the same epitope(s), except for possible variants that may arise during production of the monoclonal antibody, such variants generally being present in minor amounts. Such monoclonal antibody typically includes an antibody comprising a variable region that binds a target, wherein the antibody was obtained by a process that includes the selection of the antibody from a plurality of antibodies. For example, the selection process can be the selection of a unique clone from a plurality of clones, such as a pool of hybridoma clones, phage clones or recombinant DNA clones. It should be understood that the selected antibody can be further altered, for example, to improve affinity for the target, to humanize the antibody, to improve its production in cell culture, to reduce its immunogenicity in vivo, to create a multispecific antibody, etc., and that an antibody comprising the altered variable region sequence is also a monoclonal antibody of this disclosure. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N. Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5): 1073-1093 (2004); Fellouse, Proc. Nat. Acad. Sci. USA 101(34): 12467-12472 (2004); and Lee et al. J. Immunol. Methods 284(1-2):119-132 (2004) and technologies for producing human or human-like antibodies from animals that have parts or all of the human immunoglobulin loci or genes encoding human immunoglobulin sequences (see, e.g., WO98/24893, WO96/34096, WO96/33735, and WO91/10741, Jakobovits et al., Proc. Natl. Acad. Sci. USA, 90:2551 (1993); Jakobovits et al., Nature, 362:255-258 (1993); Bruggemann et al., Year in Immune, 7:33 (1993); U.S. Pat. Nos. 5,545,806, 5,569,825, 5,591,669 (all of GenPharm); 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

The monoclonal antibodies herein specifically include “chimeric” or “recombinant” antibodies in which a portion of the light and/or heavy chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “humanized” antibodies. In an embodiment, the antibody is a monoclonal antibody, preferably a human antibody.

The antibody of the present disclosure may be of any class or isotype, e.g., IgG, IgM, IgA, IgD or IgE. In an embodiment, the antibody of the present disclosure is an IgA. In an embodiment, the antibody of the present disclosure is an IgG. The IgG may be of any subclass, e.g., IgG1, IgG2, IgG3, or IgG4. In an embodiment, the antibody is an IgG1.

In an embodiment, the antibody of the present disclosure is a multispecific antibody or antigen-binding fragment thereof, such as a bispecific antibody or antigen-binding fragment thereof. In such multispecific (e.g., bispecific) antibodies or antigen-binding fragments thereof, at least one of the antigen-binding domains comprise one of the combinations of CDRs or variable regions described herein. In an embodiment, the multispecific (e.g., bispecific) antibody or antigen-binding fragment thereof comprises two of the combinations of CDRs or variable regions described herein. For example, the multispecific (e.g., bispecific) antibody or antigen-binding fragment thereof may comprise a first portion comprising the combinations of CDRs or variable regions from antibody Mab #1 and a second portion comprising the combinations of CDRs or variable regions from antibody Mab #25. The multispecific (e.g., bispecific) antibody or antigen-binding fragment thereof may also comprise a binding domain that binds to another antigen (i.e., other than the Spike protein from a betacoronavirus), for example a binding domain that binds to ACE2. Examples of bispecific antibody or antigen-binding fragment formats include bispecific monoclonal antibodies (mab)₂, “knob into hole” IgG, crossMab, ortho-Fab IgG, DVD-Ig, two in one IgG, IgG-scFv, scFv₂-Fc, bispecific F(mab′)₂, quadroma, bispecific diabodies (BsDb), single-chain bispecific diabodies (scBsDb), single-chain bispecific tandem variable domain (scBsTaFv), dock-and-lock trivalent Fab (DNL-(Fab)₃), bispecific single-domain antibodies (BssdAb), tandem Ab, tandem diabodies (TandAb), and tandem ScFv (see, e.g., Brinkmann and Kontermann, MAbs. 2017 February-March; 9(2): 182-212).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions (HVRs) both in the light-chain and heavy-chain variable domains. The more highly conserved portions of variable domains are called the framework region (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular cytotoxicity (ADCC). From N-terminal to C-terminal, both light and heavy chain variable regions comprise alternating FRs and CDRs: FR1, CDR1, FR2, CDR2, FR3, CDR3 and FR4. The assignment of amino acids to each region may be made in accordance with the definitions of Kabat, Chothia (Al-Lazikani et al., J Mol Biol. 1997; 273(4):927-48), or IMGT (Lefranc, M.-P., Immunology Today, 18, 509 (1997)), for example. “Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the V_{H}-V_{L} dimer. Collectively, the six CDRs are involved in conferring the antigen-binding specificity to the antibody. However, even a single variable domain (or half of an Fv comprising only three CDRs specific for an antigen) has the ability to recognize and bind antigen, although at a lower affinity than the entire binding site.

“Hypervariable region” or “HVR” refers to the amino acid residues of an antibody that are responsible for antigen-binding. The hypervariable region generally comprises amino acid residues from a “complementarity determining region” or “CDR” (Kabat et al., Sequences of Proteins of Immunological Interest, 5^{th} Ed. Public Health Service, National Institutes of Health, Bethesda, Md. (1991)) and/or those residues from a “hypervariable loop” (AI-Lazikani et al., supra).

The term “complementarity determining regions” or “CDRs” when used herein refers to parts of immunological receptors that make contact with a specific ligand and determine its specificity. The CDRs of immunological receptors are the most variable part of the receptor protein, giving receptors their diversity, and are carried on six loops at the distal end of the receptor's variable domains, three loops coming from each of the two variable domains of the receptor.

As used herein, the term “framework region” refers to those portions of immunoglobulin light and heavy chain variable regions that are relatively conserved (i.e., other than the CDRs) among different immunoglobulins in a single species, as defined by Kabat et al. (supra) or Chothia (AI-Lazikani et al., supra). As used herein, a “human framework region” is a framework region that is substantially identical to the framework region of a naturally occurring human antibody.

The sequences of the CDR and FR as defined herein are defined according to the Chothia numbering scheme. However, the skilled person would understand that the amino acids forming the CDRs and FRs regions in the sequences of the antibodies defined herein may vary depending on the numbering scheme used. Other numbering schemes include the AbM, Kabat, Contact and IMGT schemes.

In an embodiment, one or two residues in the above-noted CDRs sequences are substituted. In a further embodiment, one residue in the above-noted CDRs sequences are substituted. In another embodiment, the antibody or antigen-binding fragment thereof comprises the above-noted CDRs sequence.

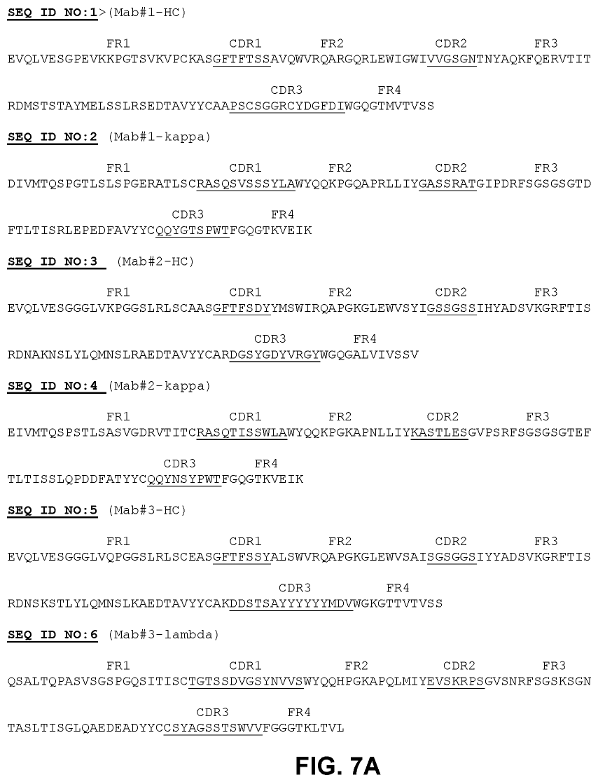

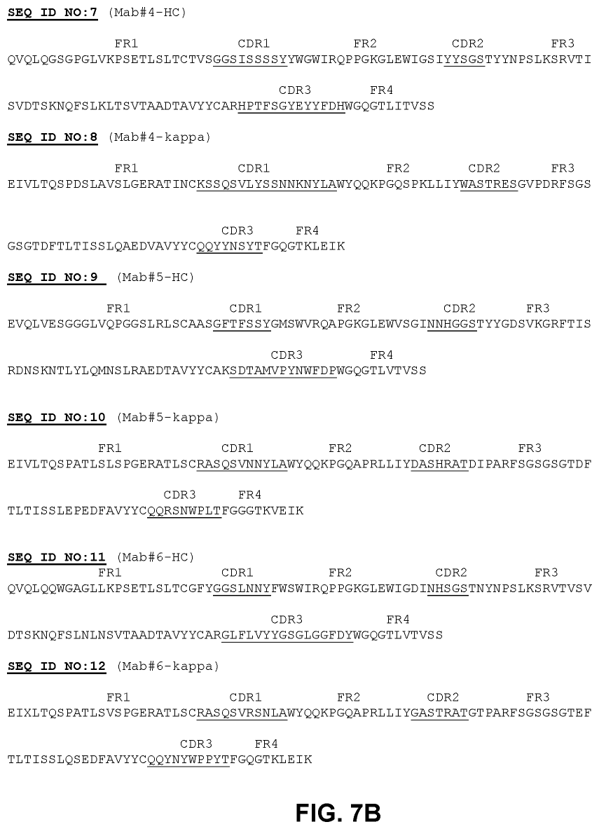

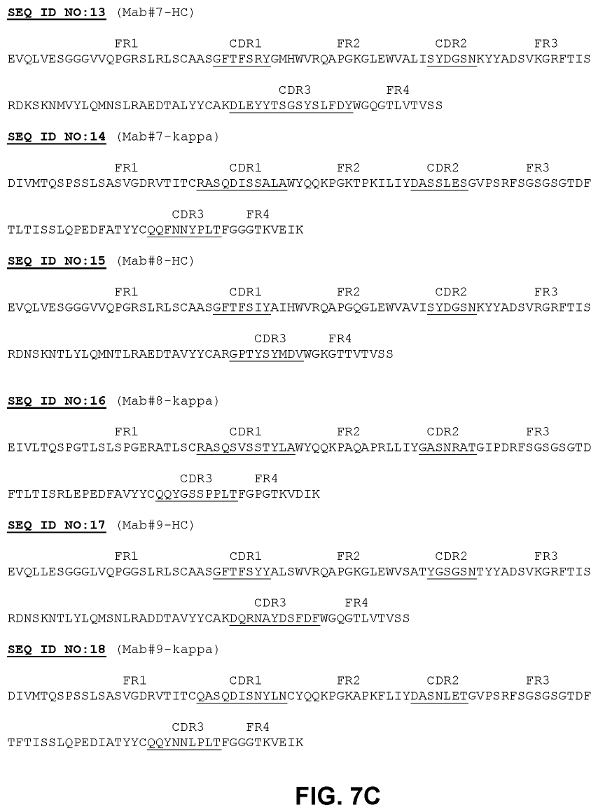

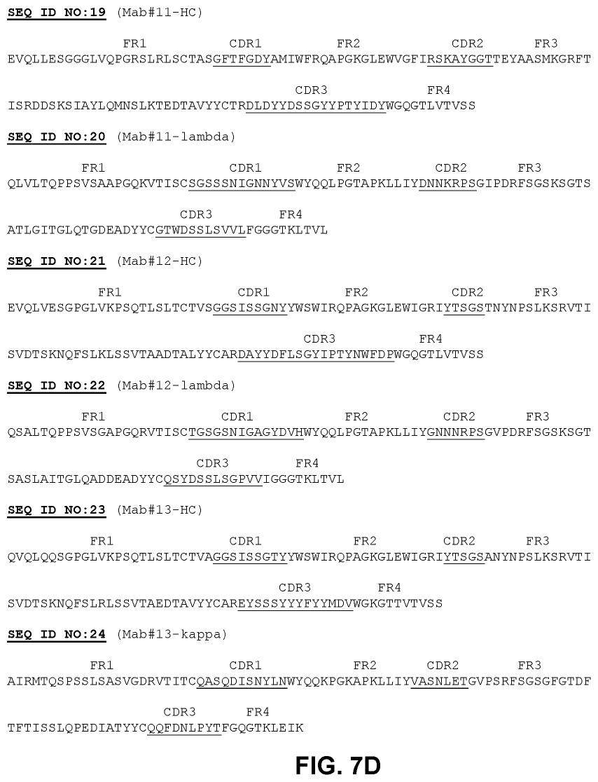

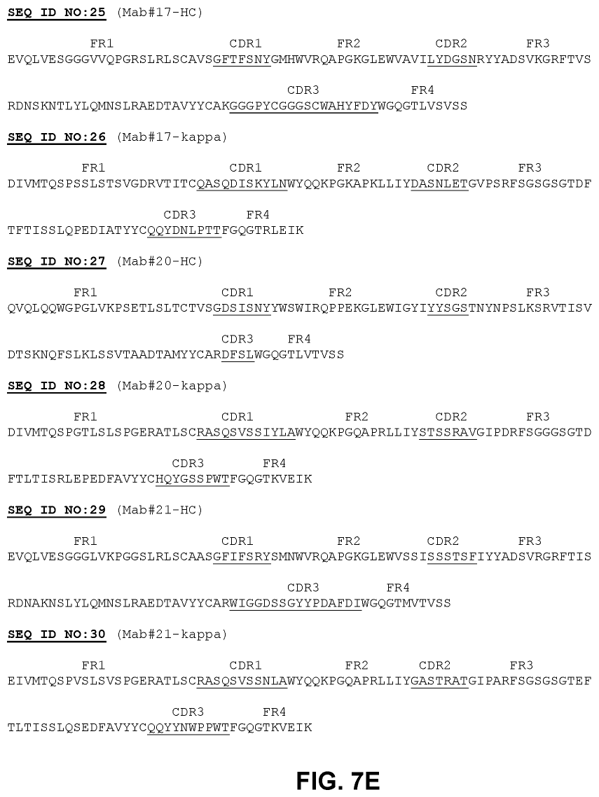

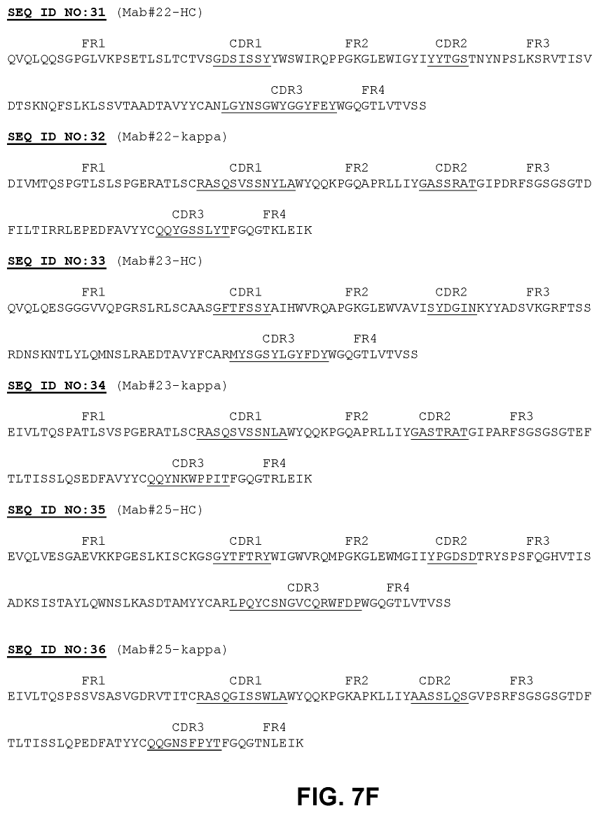

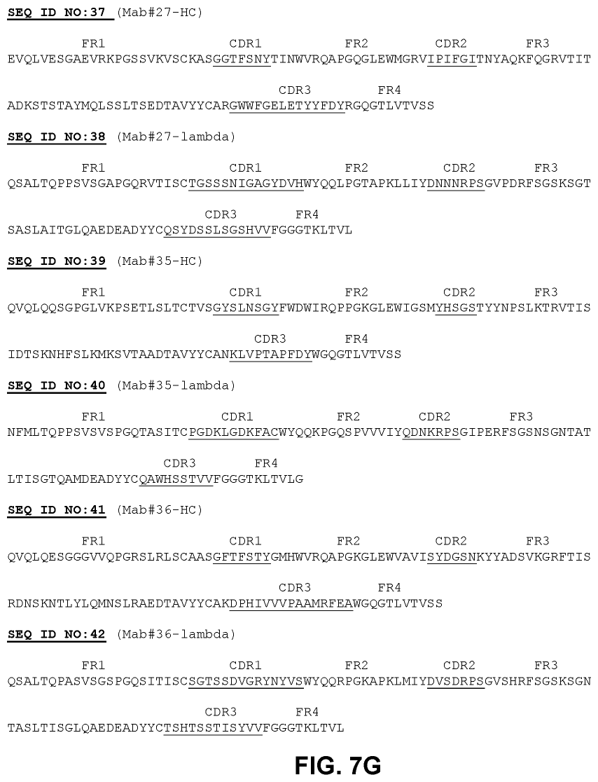

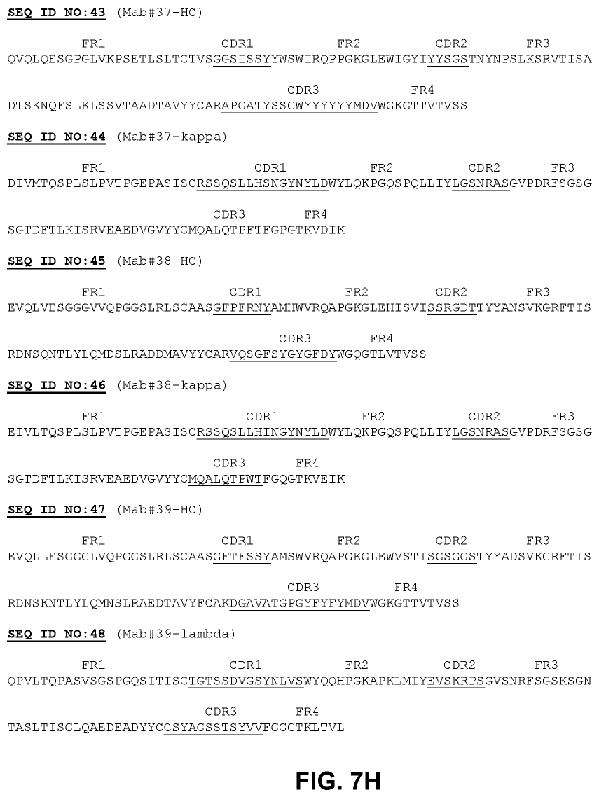

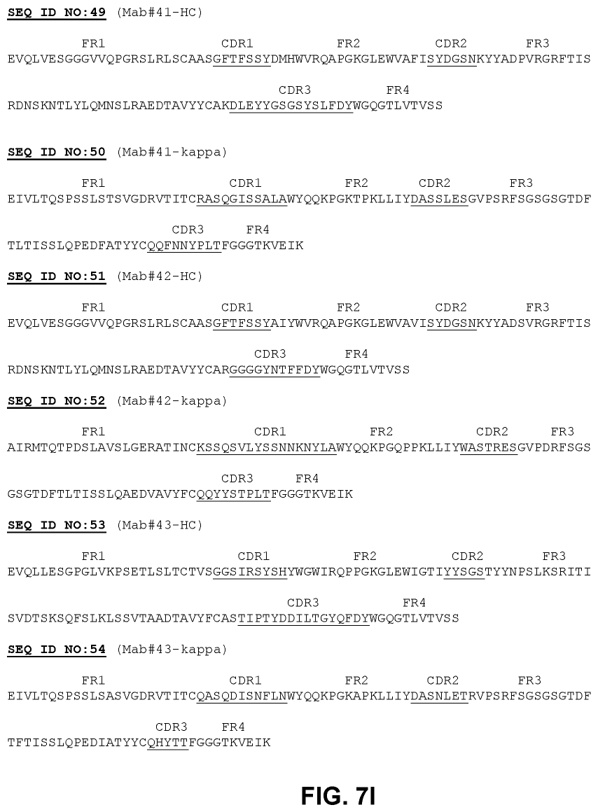

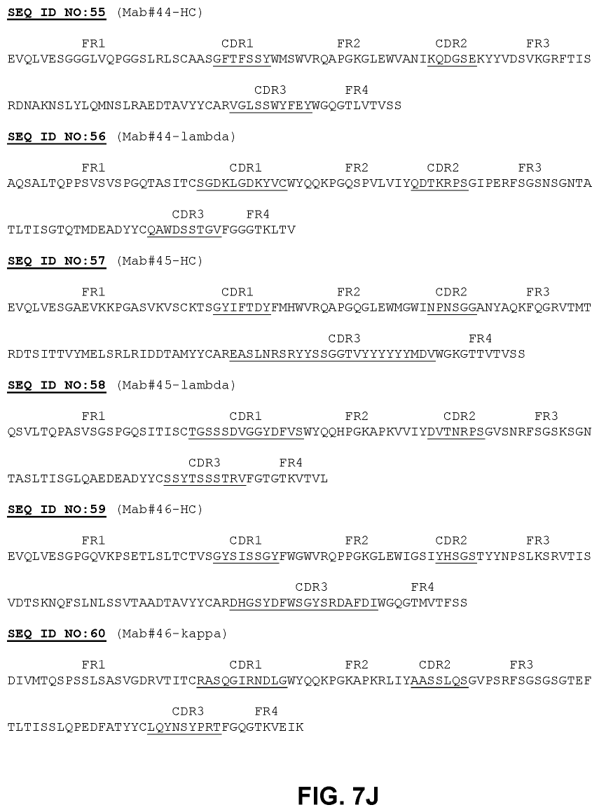

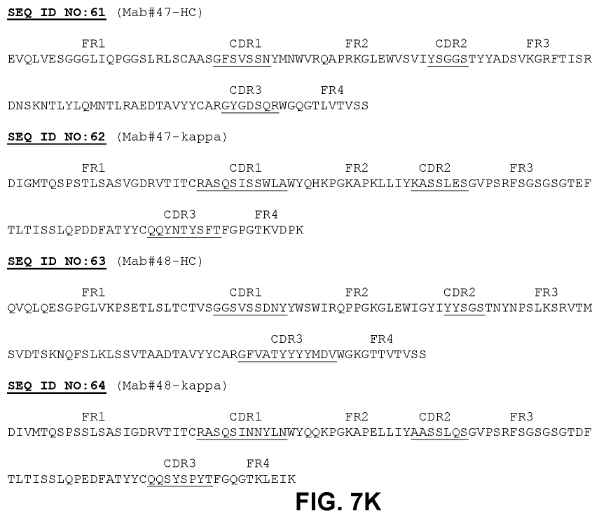

In an embodiment, the antibody or antigen-binding fragment thereof comprises a light chain FR1 comprising or consisting of an amino acid sequence having at least 50%, 60%, 70%, 75%, 80%, 85%, 90%, 95% or 100% identity with the sequences of one of the light chain FR1 depicted in FIGS. 7A-7K.

In an embodiment, the antibody or antigen-binding fragment thereof comprises a light chain FR2 comprising or consisting of an amino acid sequence having at least 50%, 60%, 70%, 75%, 80%, 85%, 90%, 95% or 100% identity with the sequences of one of the light chain FR2 depicted in FIGS. 7A-7K.

In an embodiment, the antibody or antigen-binding fragment thereof comprises a light chain FR3 comprising or consisting of an amino acid sequence having at least 50%, 60%, 70%, 75%, 80%, 85%, 90%, 95% or 100% identity with the sequences of one of the light chain FR3 depicted in FIGS. 7A-7K.

In an embodiment, the antibody or antigen-binding fragment thereof comprises a light chain FR4 comprising or consisting of an amino acid sequence having at least 50%, 60%, 70%, 75%, 80%, 85%, 90%, 95% or 100% identity with the sequences of one of the light chain FR4 depicted in FIGS. 7A-7K.

In an embodiment, the antibody or antigen-binding fragment thereof comprises a heavy chain FR1 comprising or consisting of an amino acid sequence having at least 50%, 60%, 70%, 75%, 80%, 85%, 90%, 95% or 100% identity with the sequences of one of the heavy chain FR1 depicted in FIGS. 7A-7K.

In an embodiment, the antibody or antigen-binding fragment thereof comprises a heavy chain FR2 comprising or consisting of an amino acid sequence having at least 50%, 60%, 70%, 75%, 80%, 85%, 90%, 95% or 100% identity with the sequences of one of the heavy chain FR2 depicted in FIGS. 7A-7K.

In an embodiment, the antibody or antigen-binding fragment thereof comprises a heavy chain FR3 comprising or consisting of an amino acid sequence having at least 50%, 60%, 70%, 75%, 80%, 85%, 90%, 95% or 100% identity with the sequences of one of the heavy chain FR3 depicted in FIGS. 7A-7K.

In an embodiment, the antibody or antigen-binding fragment thereof comprises a heavy chain FR4 comprising or consisting of an amino acid sequence having at least 50%, 60%, 70%, 75%, 80%, 85%, 90%, 95% or 100% identity with the sequences of one of the light heavy FR4 depicted in FIGS. 7A-7K.

In some embodiments, the antibody or antigen-binding fragment thereof comprises a heavy chain comprising a sequence having at least 70%, 75%, 80%, 85%, 90% or 95% identity with one of the following amino acid sequences: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61 and 63 (FIGS. 7A-7K), and a light chain comprising a sequence having at least 70%, 75%, 80%, 85%, 90% or 95% identity with one of the following amino acid sequences: SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62 and 64 (FIGS. 7A-7K). In an embodiment, the differences relative to the reference variable light or heavy chain sequence are within one or more of the FRs depicted in FIGS. 7A-7K. In a further embodiment, the antibody or antigen-binding fragment thereof comprises a variable light (VL) chain comprising or consisting of one of the sequences defined above. In a further embodiment, the antibody or antigen-binding fragment thereof comprises a variable heavy chain (VH) comprising or consisting of one of the sequences defined above. In an embodiment, the antibody or antigen-binding fragment thereof comprises one of the pairs of light and heavy chain variable regions depicted in FIGS. 7A-7K.

In some embodiments, the antibody or antigen-binding fragment thereof comprises a heavy chain comprising the amino acid sequence of SEQ ID NO: 1 and a light chain comprising the amino acid sequence of SEQ ID NO: 2. In some embodiments, the antibody or antigen-binding fragment thereof comprises a heavy chain comprising the amino acid sequence of SEQ ID NO: 13 and a light chain comprising the amino acid sequence of SEQ ID NO: 14. In some embodiments, the antibody or antigen-binding fragment thereof comprises a heavy chain comprising the amino acid sequence of SEQ ID NO: 25 and a light chain comprising the amino acid sequence of SEQ ID NO: 26. In some embodiments, the antibody or antigen-binding fragment thereof comprises a heavy chain comprising the amino acid sequence of SEQ ID NO: 35 and a light chain comprising the amino acid sequence of SEQ ID NO: 36. In some embodiments, the antibody or antigen-binding fragment thereof comprises a heavy chain comprising the amino acid sequence of SEQ ID NO: 53 and a light chain comprising the amino acid sequence of SEQ ID NO: 54. In some embodiments, the monoclonal antibody comprises a heavy chain comprising the amino acid sequence of SEQ ID NO: 57 and a light chain comprising the amino acid sequence of SEQ ID NO: 58.

Variations in the antibodies or antigen-binding fragments thereof described herein, can be made, for example, using any of the techniques and guidelines for conservative and non-conservative mutations set forth, for instance, in U.S. Pat. No. 5,364,934. Variations may be a substitution, deletion or insertion of one or more codons encoding the antibody that results in a change in the amino acid sequence as compared with the native sequence antibody. Optionally the variation is by substitution of at least one amino acid with any other amino acid in one or more of the domains of the antibody or antigen-binding fragment thereof. Guidance in determining which amino acid residue may be inserted, substituted or deleted without adversely affecting the desired activity may be found by comparing the sequence of the antibody or antigen-binding fragment thereof with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence. In embodiment, the variant exhibits at least 50%, 55% or 60%, preferably at least 65, 70, 75, 80, 90, 95, 96, 97, 98 or 99% sequence identity with the sequence of the antibody or antigen-binding fragment thereof described herein, and maintains the ability to specifically bind to SARS-CoV-2 Spike protein and/or to neutralize SARS-CoV-2 infection.

“Identity” refers to sequence identity between two polypeptides. Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Covalent modifications of antibodies or antigen-binding fragments thereof are included within the scope of this disclosure. Covalent modifications include reacting targeted amino acid residues of the antibody or antigen-binding fragment thereof with an organic derivatizing agent that is capable of reacting with selected side chains or the N- or C-terminal residues of the antibody or antigen-binding fragment thereof. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Other types of covalent modification of the antibody or antigen-binding fragment thereof included within the scope of this disclosure include altering the native glycosylation pattern of the antibody or antigen-binding fragment thereof (Beck et al., Curr. Pharm. Biotechnol. 9: 482-501, 2008; Walsh, Drug Discov. Today 15: 773-780, 2010), and linking the antibody or antigen-binding fragment thereof to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The antibody or antigen-binding fragment thereof may further comprise one or more modifications that confer additional biological properties to the antigenic peptide such as protease resistance, plasma protein binding, increased plasma half-life, intracellular penetration, etc. Such modifications include, for example, covalent attachment of molecules/moiety to the antibody or antigen-binding fragment thereof such as fatty acids (e.g., C₆-C₁₈), attachment of proteins such as albumin (see, e.g., U.S. Pat. No. 7,268,113); sugars/polysaccharides (glycosylation), biotinylation or PEGylation (see, e.g., U.S. Pat. Nos. 7,256,258 and 6,528,485). The above description of modification of the antigenic peptide does not limit the scope of the approaches nor the possible modifications that can be engineered. Thus, in another aspect, the present disclosure provides a conjugate comprising the antibody or antigen-binding fragment thereof described herein and one or more additional molecules or agents (hereinafter secondary molecules or agents). The antigenic peptide may be conjugated to any type of synthetic or natural secondary molecules or agents, such as peptides. proteins, saccharides/polysaccharides, lipids, naturally-occurring or synthetic polymers/co-polymers, etc. to modify one or more properties of the antibody or antigen-binding fragment thereof.

In an embodiment, the conjugate comprises a covalent link or bond between the antigenic peptide and the molecule conjugated thereto. The molecule may be conjugated directly to the antigenic peptide, or indirectly via a linker. The linker may be a polypeptide linker comprising one or more amino acids or another type of chemical linker (e.g., a carbohydrate linker, a lipid linker, a fatty acid linker, a polyether linker, PEG, etc.

In another embodiment, the molecule may be conjugated/attached to the side chain of one the amino acids of the antibody or antigen-binding fragment thereof. Methods for conjugating moieties to side chains of amino acids are well known in the art. For example, chemical groups that react with primary amines (—NH₂) present in the side-chain of lysine residues such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters may be used to conjugate the molecule to the antigenic peptide. Most of these groups conjugate to amines by either acylation or alkylation. Cysteine residues present in the antibody or antigen-binding fragment thereof may also be used to attach the molecule.

In an embodiment, the antibody or antigen-binding fragment thereof is labelled or conjugated with one or more moieties. The antibody or antigen-binding fragment thereof may be labeled with one or more labels such as a biotin label, a fluorescent label, an enzyme label, a coenzyme label, a chemiluminescent label, or a radioactive isotope label. In an embodiment, the antibody or antigen-binding fragment thereof is labelled with a detectable label, for example a fluorescent moiety (fluorophore). Useful detectable labels include fluorescent compounds (e.g., fluorescein isothiocyanate, Texas red, rhodamine, fluorescein, Alexa Fluor® dyes, and the like), radiolabels, enzymes (e.g., horseradish peroxidase, alkaline phosphatase and others commonly used in a protein detection assays), streptavidin/biotin, and colorimetric labels such as colloidal gold, colored glass or plastic beads (e.g., polystyrene, polypropylene, latex, etc.). Chemiluminescent compounds may also be used. Such labelled antibodies or antigen-binding fragments thereof may be useful, for example, for the detection of SARS-CoV-2 and/or SARS-CoV-2-infected cells in vivo or in vitro, e.g., by flow cytometry, immunohistochemistry, etc. The antibody or antigen-binding fragment thereof can also be conjugated to detectable or affinity tags that facilitate detection and/or purification of the antibody or antigen-binding fragment thereof. Such tags are well known in the art. Examples of detectable or affinity tags include polyhistidine tags (His-tags), polyarginine tags, polyaspartate tags, polycysteine tags, polyphenylalanine tags, glutathione S-transferase (GST) tags, Maltose binding protein (MBP) tags, calmodulin binding peptide (CBP) tags, Streptavidin/Biotin-based tags, HaloTag®, Profinity eXact® tags, epitope tags (such as FLAG, hemagglutinin (HA), HSV, S/S1, c-myc, KT3, T7, V5, E2, and Glu-Glu epitope tags), reporter tags such as β-galactosidase (β-gal), alkaline phosphatase (AP), chloramphenicol acetyl transferase (CAT), and horseradish peroxidase (HRP) tags (see, e.g., Kimple et al., Curr Protoc Protein Sci. 2013; 73: Unit-9.9).

In some embodiments, the antibody or antigen-binding fragment thereof described herein binds to an epitope in the viral envelope spike protein (S) of a betacoronavirus, such as a sarbecovirus, e.g., SARS-CoV-2. In some embodiments, the antibody or antigen-binding fragment thereof binds to an epitope in the receptor binding domain (RBD) of a betacoronavirus, such as a sarbecovirus, e.g., SARS-CoV-2 S protein. In some embodiments, the antibody or antigen-binding fragment thereof binds to an epitope that is outside the RBD of a betacoronavirus, such as a sarbecovirus, e.g., SARS-CoV-2 S protein. In some embodiments, the antibody or antigen-binding fragment thereof neutralizes a betacoronavirus, such as a sarbecovirus, e.g., SARS-CoV-2. In some embodiments, the antibody or antigen-binding fragment thereof inhibits viral and cell membrane fusion. In some embodiments, the antibody or antigen-binding fragment thereof binds to the S1 subunit of a betacoronavirus, such as a sarbecovirus, e.g., SARS-CoV-2 S protein. In some embodiments, the antibody or antigen-binding fragment thereof binds to the S2 subunit of a betacoronavirus, such as a sarbecovirus, e.g., SARS-CoV-2 S protein. In some embodiments, the antibody or antigen-binding fragment thereof also binds to the S protein of one at least two betacoronaviruses, such as SARS-CoV-1 and SARS-CoV-2.

The amino acid sequence of the full-length Spike protein from SARS-CoV-2 (Wuhan strain, NCBI Reference Sequence YP_009724390.1, SEQ ID NO: 237) is depicted below:

| | |
|---|---|
| 1 | MFVFLVLLPL VSSQCVNLTT RTQLPPAYTN SFTRGVYYPD KVFRSSVLHS TQDLFLPFFS |
| 61 | NVTWFHAIHV SGTNGTKRFD NPVLPFNDGV YFASTEKSNI IRGWIFGTTL DSKTQSLLIV |
| 121 | NNATNVVIKV CEFQFCNDPF LGVYYHKNNK SWMESEFRVY SSANNCTFEY VSQPFLMDLE |
| 181 | GKQGNFKNLR EFVFKNIDGY FKIYSKHTPI NLVRDLPQGF SALEPLVDLP IGINITRFQT |
| 241 | LLALHRSYLT PGDSSSGWTA GAAAYYVGYL QPRTFLLKYN ENGTITDAVD CALDPLSETK |
| 301 | CTLKSFTVEK GIYQTSNFRV QPTESIVRFP NITNLCPFGE VFNATRFASV YAWNRKRISN |
| 361 | CVADYSVLYN SASFSTFKCY GVSPTKLNDL CFTNVYADSF VIRGDEVRQI APGQTGKIAD |
| 421 | YNYKLPDDFT GCVIAWNSNN LDSKVGGNYN YLYRLFRKSN LKPFERDIST EIYQAGSTPC |
| 481 | NGVEGFNCYF PLQSYGFQPT NGVGYQPYRV VVLSFELLHA PATVCGPKKS TNLVKNKCVN |
| 541 | FNFNGLTGTG VLTESNKKFL PFQQFGRDIA DTTDAVRDPQ TLEILDITPC SFGGVSVITP |
| 601 | GTNTSNQVAV LYQDVNCTEV PVAIHADQLT PTWRVYSTGS NVFQTRAGCL IGAEHVNNSY |
| 661 | ECDIPIGAGI CASYQTQTNS PRRARSVASQ SIIAYTMSLG AENSVAYSNN SIAIPTNFTI |
| 721 | SVTTEILPVS MTKTSVDCTM YICGDSTECS NLLLQYGSFC TQLNRALTGI AVEQDKNTQE |
| 781 | VFAQVKQIYK TPPIKDFGGF NFSQILPDPS KPSKRSFIED LLFNKVTLAD AGFIKQYGDC |
| 841 | LGDIAARDLI CAQKFNGLTV LPPLLTDEMI AQYTSALLAG TITSGWTFGA GAALQIPFAM |
| 901 | QMAYRFNGIG VTQNVLYENQ KLIANQFNSA IGKIQDSLSS TASALGKLQD VVNQNAQALN |
| 961 | TLVKQLSSNF GAISSVLNDI LSRLDKVEAE VQIDRLITGR LQSLQTYVTQ QLIRAAEIRA |
| 1021 | SANLAATKMS ECVLGQSKRV DFCGKGYHLM SFPQSAPHGV VFLHVTYVPA QEKNFTTAPA |
| 1081 | ICHDGKAHFP REGVFVSNGT HWFVTQRNFY EPQIITTDNT FVSGNCDVVI GIVNNTVYDP |
| 1141 | LQPELDSFKE ELDKYFKNHT SPDVDLGDIS GINASVVNIQ KEIDRLNEVA KNLNESLIDL |
| 1201 | QELGKYEQYI KWPWYIWLGF IAGLIAIVMV TIMLCCMTSC CSCLKGCCSC GSCCKFDEDD |
| 1261 | SEPVLKGVKL HYT |

Residues 1-12 correspond to the signal peptide, residues 13-685 correspond to the Spike protein subunit S1 and residues 686 correspond to the Spike protein subunit S2. The receptor-binding domain (RBD) is defined by residues 319-541 (receptor-binding motif=residues 437-508). Residues 816-837 define the fusion peptide 1, residues 835-855 define the fusion peptide 2, residues 920-970 define the heptad repeat 1 and residues 1163-1202 define the heptad repeat 2.

SARS-CoV2 variants comprise mutations in the Spike protein including L5F, S131, L18F, T19R, T20N, P26S, A67V, del69-70, G75V, T761, D80Y, D80A, T951, S98F, R102I, D138Y, G142D, del142-144, del144, W152C, E154K, EFR156-158G, F157L, R190S, ins214EPE, D215G, A222V, del246-252, D253G, W258L, N354D, F342L, V367F, K417N, K417T, A435S, W436R, N439K, N440K, G446V, L452R, Y453F, K458R, G476S, S477N, S477G, T478K, V483A, E484K, E484Q, F490S, N501Y, N501S, N501T, A570D, Q613H, D614G, A626S, A653V, H655Y, Q677H, Q677P, P681H, P681R, A701V, T716I, D796H, D796Y, T859N, F888L, D950N, S982A, T1027I, Q1071H, E1092K, H1101Y, D1118H, V1176F, G1219V, and V1122L.

The Delta variant comprises the following Spike protein mutations: T19R, (V70F*), T951, G142D, E156−, F157−, R158G, (A222V*), (W258L*), (K417N*), L452R, T478K, D614G, P681R, D950N.

The Omicron variant comprises the following Spike protein mutations: A67V, del69-70, T951, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F.

In an embodiment, the antibody or antigen-binding fragment thereof described herein binds to the Spike protein from a SARS-CoV2 variant. In an embodiment, the antibody or antigen-binding fragment thereof described herein binds to the Spike protein from the SARS-CoV2 Delta variant. In an embodiment, the antibody or antigen-binding fragment thereof described herein binds to the Spike protein from the SARS-CoV2 Omicron variant.

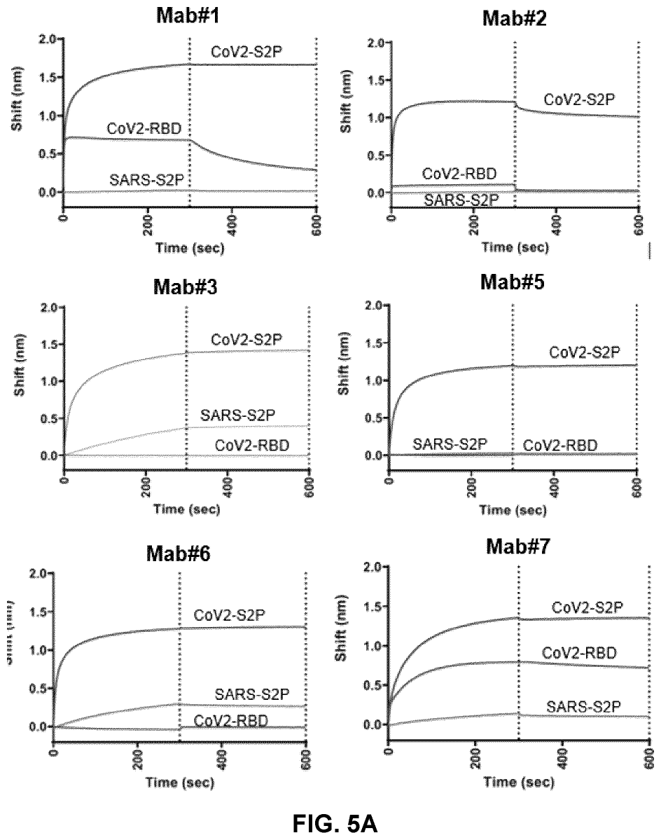

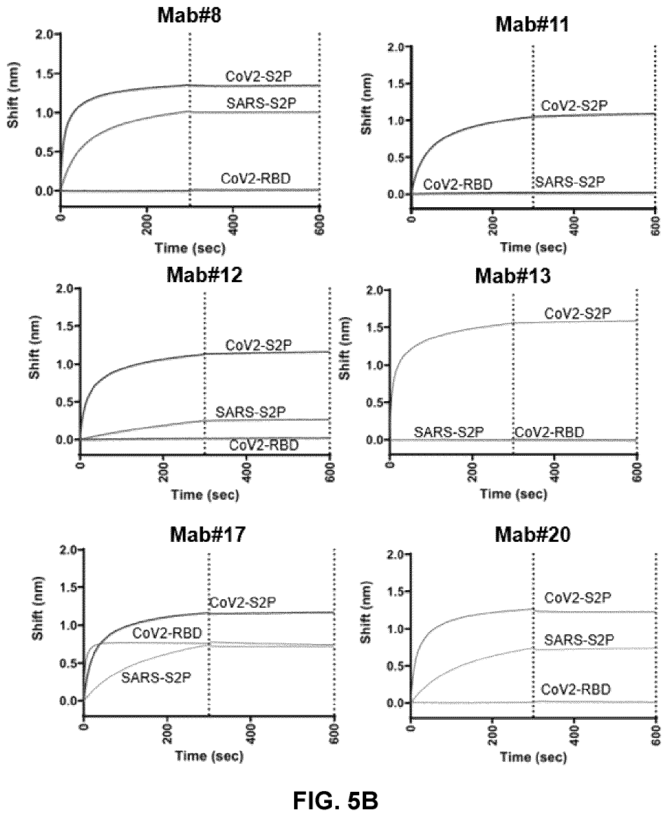

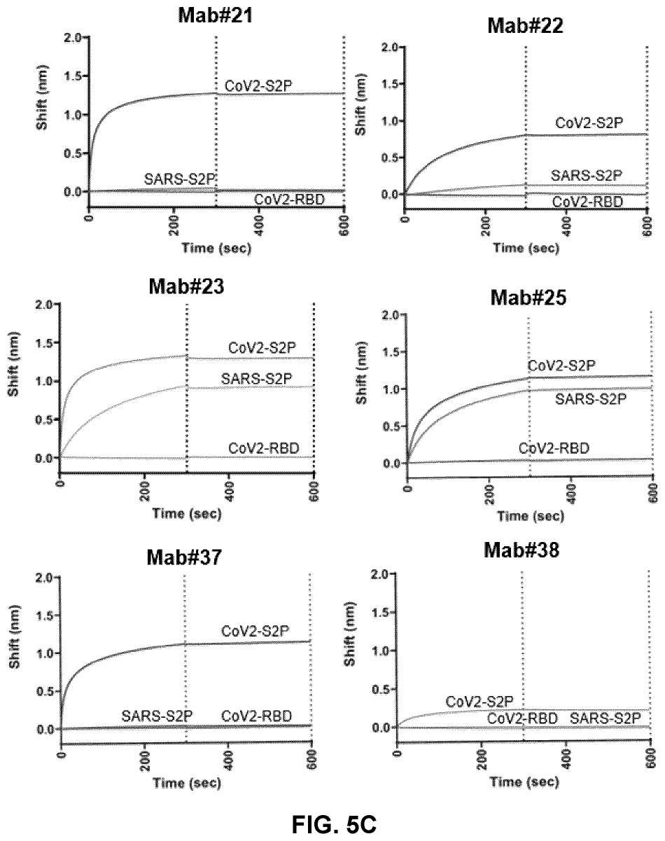

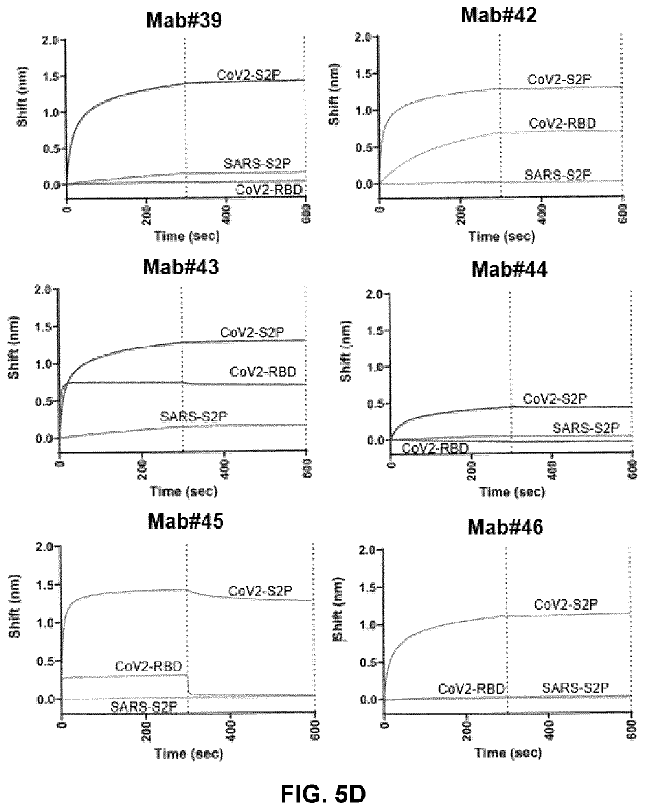

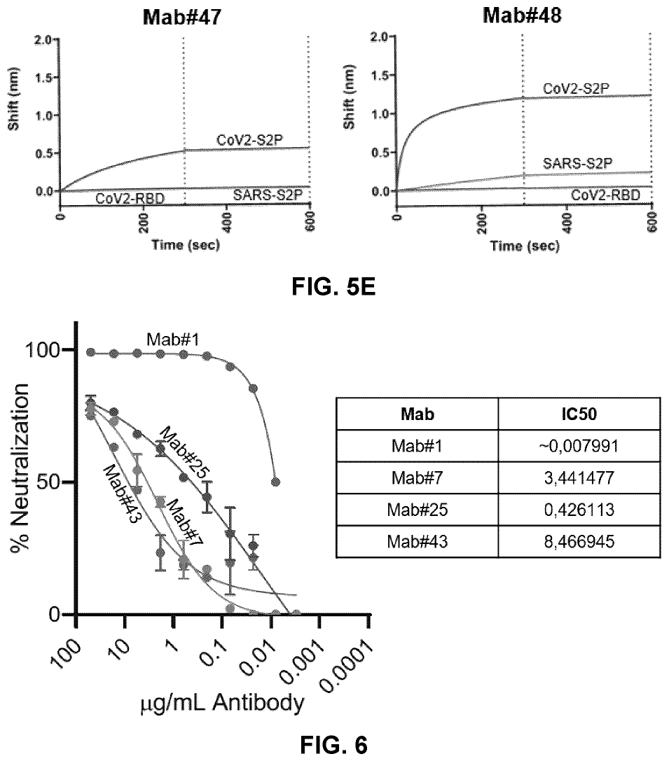

In another aspect, the present disclosure provides a combination of at least two antibodies or antigen-binding fragments thereof described herein. In an embodiment, the combination comprises a first antibody or antigen-binding fragment thereof that binds to the S1 subunit (e.g. in the RBD) of SARS-CoV-2 S protein, and a second antibody or antigen-binding fragment thereof that binds outside the RBD of SARS-CoV-2 S protein, for example that binds to the S2 subunit of SARS-CoV-2 S protein. Examples of antibodies that binds to the RBD of SARS-CoV-2 S protein include Mab #1, Mab #7, Mab #17, and Mab #43 (FIGS. 5A-E), and thus the first antibody or antigen-binding fragment thereof may comprise amino acid sequences having at least 70%, 75%, 80%, 85%, 90%, 95% or 100% identity with the CDRs of these antibodies as depicted in FIGS. 7A-K. In a further embodiment, the antibody or antigen-binding fragment thereof comprises the amino acid sequences of the CDRs of antibody Mab #1 (FIG. 7A). In a further embodiment, the antibody or antigen-binding fragment thereof comprises the amino acid sequences of the heavy and light chain variable regions of antibody Mab #1 (SEQ ID NOs: 1 and 2, FIG. 7A). Examples of antibodies that binds outside the RBD of SARS-CoV-2 S protein include Mab #2, Mab #3, Mab #5, Mab #6, Mab #8, Mab #11, Mab #12, Mab #13, Mab #20, Mab #21, Mab #22, Mab #23, Mab #25, Mab #37, Mab #38, Mab #39, Mab #42, Mab #44, Mab #45, Mab #46, Mab #47, and Mab #48 (FIGS. 5A-E), and thus the first antibody or antigen-binding fragment thereof may comprise amino acid sequences having at least 70%, 75%, 80%, 85%, 90%, 95% or 100% identity with the CDRs of these antibodies as depicted in FIGS. 7A-K. In a further embodiment, the antibody or antigen-binding fragment thereof comprises the amino acid sequences of the CDRs of antibody Mab #25 (FIG. 7F). In a further embodiment, the antibody or antigen-binding fragment thereof comprises the amino acid sequences of the heavy and light chain variable regions of antibody Mab #25 (SEQ ID NOs: 35 and 36, FIG. 7F).

A further aspect of the present disclosure provides nucleic acids encoding the antibody or antigen-binding fragment described herein, e.g., encoding the light and heavy chains of the antibody or antigen-binding fragment. The isolated nucleic acid may be a synthetic DNA, a mRNA (e.g., a non-naturally occurring mRNA), or a cDNA, for example. The nucleic acid may be inserted within a plasmid, vector, or transcription or expression cassette. The nucleic acids encoding the antibody or antigen-binding fragment described herein may be made and the expressed antibodies or antigen-binding fragments described may be tested using conventional techniques well known in the art. In some embodiments, the nucleic acid encoding the antibody or antigen-binding fragment described herein can be maintained in the vector in a host cell. In another aspect, provided herein is a nucleic acid comprising a sequence encoding the amino acid sequence of any one of SEQ ID NOs: 1-64. In some embodiments, the nucleic acid is an expression vector. In some embodiments, the nucleic acid sequence encoding the antibody can be maintained in the vector in a host cell. In embodiment, the nucleic acid(s) (DNA, mRNA) encoding the antibody or antigen-binding fragment described herein is/are comprised within a vesicles such as lipid nanoparticles (e.g., liposomes) or any other suitable vehicle. In an embodiment, the nucleic acid(s) is/are mRNA and is/are encapsulated into nanoparticulate delivery vehicles (see, e.g., Van Hoecke and Roose, Journal of Translational Medicine, volume 17, Article number: 54 (2019); Sanz and Álvarez-Vallina, Antibodies (Basel). 2021 Sep. 26; 10(4):37).

In another aspect, the present disclosure provides a cell, for example a recombinant host cell, expressing the antibody or antigen-binding fragment described herein. Methods of preparing antibodies or antigen-binding fragments comprise expressing the encoding nucleic acid(s) in a host cell under conditions to produce the antibodies or antigen-binding fragments, and recovering the antibodies or antigen-binding fragments. The process of recovering the antibodies or antigen-binding fragments may comprise isolation and/or purification of the antibodies or antigen-binding fragments. The method of production may comprise formulating the antibodies or antigen-binding fragments into a composition including at least one additional component, such as a pharmaceutically acceptable excipient. In another aspect, provided herein is a cell expressing one or more antibodies of the disclosure. In some embodiments, the cell comprises one or more nucleic acid sequences encoding the amino acid sequence of any one of SEQ ID NOs: 1-64.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. To produce the antibody or antigen-binding fragment thereof recombinantly, the nucleic acid or nucleic acids encoding the light and heavy chains of the antibody or antigen-binding fragment thereof are introduced in a cell which is able to produce the recombinant antibody. Examples thereof include CHO-K1 (ATCC CCL-61), DUkXB11 (ATCC CCL-9096), Pro-5 (ATCC CCL-1781), CHO-S(Life Technologies®, Cat #11619), rat myeloma cell YB2/3HL.P2.G11.16Ag.20 (also called YB2/0), mouse myeloma cell NSO, mouse myeloma cell SP2/0-Ag14 (ATCC No. CRL1581), mouse P3-X63-Ag8653 cell (ATCC No. CRL1580), CHO cell in which a dihydrofolate reductase gene is defective, lectin resistance-acquired Lec13, CHO cell in which α1,6-fucosyltransaferse gene is defective, rat YB2/3HL.P2.G11.16Ag.20 cell (ATCC No. CRL1662), CHO-3E7 cells (expressing a truncated but functional form of EBNA1, U.S. Pat. No. 8,637,315) or the like. After introduction of the expression vector, transformants which stably express a recombinant antibody are selected by culturing them in a medium for animal cell culture containing an agent such as G418 sulfate or the like. Examples of the medium for animal cell culture include RPM11640 medium (manufactured by Invitrogen®), GIT medium (manufactured by Nihon Pharmaceutical®), EX-CELL301@ medium (manufactured by JRH®), IMDM medium (manufactured by Invitrogen®), Hybridoma-SFM medium (manufactured by Invitrogen®), media obtained by adding various additives such as FBS to these media, or the like. The recombinant antibody can be produced and accumulated in a culture supernatant by culturing the obtained transformants in a medium. The expression level and antigen binding activity of the recombinant antibody in the culture supernatant can be measured by ELISA or the like. Also, in the transformant, the expression level of the recombinant antibody can be increased by using DHFR amplification system or the like. The recombinant antibody can be purified from the culture supernatant of the transformant by using a protein A column. In addition, the recombinant antibody can be purified by combining the protein purification methods such as gel filtration, ion-exchange chromatography, ultrafiltration or the like. The molecular weight of the H chain or the L chain of the purified recombinant antibody or the antibody molecule as a whole is determined by polyacrylamide gel electrophoresis, Western blotting, or the like.

Suitable vectors comprising nucleic acid(s) encoding the antibody or antigen-binding fragment described herein can be chosen or constructed, containing appropriate regulatory sequences, including promoter sequences, terminator sequences, polyadenylation sequences, enhancer sequences, marker genes and other sequences as appropriate. Vectors may be plasmids, phage, phagemids, adenoviral, AAV, lentiviral, for example. Techniques and protocols for manipulation of nucleic acid, for example in preparation of nucleic acid constructs, mutagenesis, sequencing, introduction of DNA into cells, and gene expression, are well known in the art.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

Introducing such nucleic acids into a host cell can be accomplished using techniques well known in the art. For eukaryotic cells, suitable techniques may include calcium phosphate transfection, DEAE-Dextran, electroporation, liposome-mediated transfection, and transduction using retroviruses or other viruses, for example. For bacterial cells, suitable techniques may include calcium chloride transformation, electroporation, and transfection using bacteriophage. The introduction may be followed by causing or allowing expression from the nucleic acid, e.g. by culturing host cells under conditions for expression of the gene. In one embodiment, the nucleic acid of the invention is integrated into the genome, e.g., chromosome, of the host cell. Integration may be promoted by inclusion of sequences which promote recombination with the genome, in accordance with standard techniques.

In another aspect, the present disclosure provides a composition comprising the antibody or antigen-binding fragment thereof defined herein, or the nucleic acid(s) encoding the antibody or antigen-binding fragment thereof. In an embodiment, the composition further comprises the above-mentioned antibody or an antigen-binding fragment thereof, or the nucleic acid(s) encoding the antibody or antigen-binding fragment thereof, and a carrier or excipient, in a further embodiment a pharmaceutically acceptable carrier or excipient. Such compositions may be prepared in a manner well known in the pharmaceutical art by mixing the antibody or an antigen-binding fragment thereof, or the nucleic acid(s) encoding the antibody or antigen-binding fragment thereof, having a suitable degree of purity with one or more optional pharmaceutically acceptable carriers or excipients (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). The carrier/excipient can be suitable for administration of the antibody or an antigen-binding fragment thereof, or the nucleic acid(s) encoding the antibody or antigen-binding fragment thereof, by any conventional administration route, for example, for oral, intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration. In an embodiment, the carrier/excipient is adapted for administration of the antibody or an antigen-binding fragment thereof by the intravenous or subcutaneous route. In an embodiment, the carriers/excipients are adapted for administration of the antibody or an antigen-binding fragment thereof, or the nucleic acid(s) encoding the antibody or antigen-binding fragment thereof, by the intravenous route. In another embodiment, the carriers/excipients are adapted for administration of the antibody or an antigen-binding fragment thereof, or the nucleic acid(s) encoding the antibody or antigen-binding fragment thereof, by the subcutaneous route. In an embodiment, the carriers/excipients are adapted for administration of the antibody or an antigen-binding fragment thereof, or the nucleic acid(s) encoding the antibody or antigen-binding fragment thereof, by the pulmonary route.

An “excipient” as used herein has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients (the antibody or an antigen-binding fragment thereof, or the nucleic acid(s) encoding the antibody or antigen-binding fragment thereof) and that is not toxic to the subject, i.e., is a type of excipient and/or is for use in an amount which is not toxic to the subject. Excipients are well known in the art, and the present system is not limited in these respects. In certain embodiments, one or more formulations of the dosage form include excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. Examples of commonly used excipient include water, saline, phosphate buffered saline, dextrose, glycerol, ethanol, and the like, as well as combinations thereof. In many cases, it will be preferable to include isotonic agents, for example, sugars, polyalcohols, such as mannitol, sorbitol, or sodium chloride in the composition. Additional examples of pharmaceutically acceptable substances are wetting agents or auxiliary substances, such as emulsifying agents, preservatives, or buffers, which increase the shelf life or effectiveness. In an embodiment, the antibody or antigen-binding fragment thereof defined herein, or the nucleic acid(s) encoding the antibody or antigen-binding fragment thereof, is/are encapsulated in a vesicle or vesicle-like particle, such as a lipid vesicle (e.g., liposome). The term “lipid vesicle” (or “lipid-based vesicle”) as used herein encompasses macromolecular structures which as the main constituent include lipid or lipid derivatives. Suitable examples hereof are liposomes and micelles including detergent micelles/lipid emulsion, liposomes prepared from palmitoyloleoylphosphatidylcholine, hydrogenated soy phosphatdylcholine, and solid lipid nanoparticles prepared from steric acid or tripalmitin. The term liposome is used herein in accordance with its usual meaning, referring to microscopic lipid vesicles composed of a bilayer of phospholipids or any similar amphipathic lipids encapsulating an internal aqueous medium. The liposomes may be unilamellar vesicles such as small unilamellar vesicles (SUVs), which typically have a diameter of less than 0.2 μm (e.g., between 0.02 and 0.2 μm), and large unilamellar vesicles (LUVs), and multilamellar vesicles (MLV), which typically have a diameter greater than 0.45 μm (in some cases greater than 1 μm). No particular limitation is imposed on the liposomal membrane structure in the present disclosure. The term liposomal membrane refers to the bilayer of phospholipids separating the internal aqueous medium from the external aqueous medium.

The composition may also comprise one or more additional active agents for the treatment the targeted disease/condition or for the management of symptom(s) of the targeted disease/condition (e.g., pain killers, anti-nausea agents, anti-inflammatory agents, immunotherapeutic agents, etc.).

In another aspect, the present disclosure provides a method for preventing a betacoronavirus, such as a sarbecovirus, e.g., SARS-CoV-2 infection or a related disease (Coronavirus disease 2019, COVID-19), in a subject in need thereof, the method comprising administering to the subject an effective amount of the antibody or antigen-binding fragment thereof, of one more nucleic acids encoding the antibody antigen-binding fragment thereof, or pharmaceutical composition described herein. The present disclosure also provides the use of the antibody or antigen-binding fragment thereof, of one more nucleic acids encoding the antibody antigen-binding fragment thereof, or pharmaceutical composition described herein, for preventing a betacoronavirus, such as a sarbecovirus, e.g., SARS-CoV-2 infection or a related disease (e.g., COVID-19) in a subject. The present disclosure also provides the use of the antibody or antigen-binding fragment thereof, of one more nucleic acids encoding the antibody antigen-binding fragment thereof, or pharmaceutical composition described herein, for the manufacture of a medicament for preventing a betacoronavirus, such as a sarbecovirus, e.g., SARS-CoV-2 infection or a related disease (e.g., COVID-19) in a subject.

In another aspect, the present disclosure provides a method for reducing the risk of developing a betacoronavirus-related disease, such as a sarbecovirus-related disease (e.g., COVID-19), or the severity of a betacoronavirus-related disease, such as a sarbecovirus-related disease (e.g., COVID-19), in a subject in need thereof, the method comprising administering to the subject an effective amount of the antibody or antigen-binding fragment thereof, of one more nucleic acids encoding the antibody antigen-binding fragment thereof, or pharmaceutical composition described herein. The present disclosure also provides the use of the antibody or antigen-binding fragment thereof, of one more nucleic acids encoding the antibody antigen-binding fragment thereof, or pharmaceutical composition described herein, for reducing the risk of developing a betacoronavirus-related disease, such as a sarbecovirus-related disease (e.g., COVID-19), or the severity of a betacoronavirus-related disease, such as a sarbecovirus-related disease (e.g., COVID-19), in a subject. The present disclosure also provides the antibody or antigen-binding fragment thereof, of one more nucleic acids encoding the antibody antigen-binding fragment thereof, or pharmaceutical composition described herein, for use in reducing the risk of developing a betacoronavirus-related disease, such as a sarbecovirus-related disease (e.g., COVID-19), or the severity of a betacoronavirus-related disease, such as a sarbecovirus-related disease (e.g., COVID-19), in a subject.

In another aspect, the present disclosure provides a method (in vitro or in vivo) for blocking the entry of a betacoronavirus, such as a sarbecovirus, e.g., SARS-CoV-2 in a cell, such as an ACE2-expressing cell, comprising contacting the cell and/or virus with an effective amount of the antibody or antigen-binding fragment thereof, of one more nucleic acids encoding the antibody antigen-binding fragment thereof, or pharmaceutical composition described herein. The present disclosure provides the use of the antibody or antigen-binding fragment thereof, of one more nucleic acids encoding the antibody antigen-binding fragment thereof, or pharmaceutical composition described herein, for blocking the entry of a betacoronavirus, such as a sarbecovirus, e.g., SARS-CoV-2 in a cell, such as an ACE2-expressing cell. The present disclosure provides the use of the antibody or antigen-binding fragment thereof, of one more nucleic acids encoding the antibody antigen-binding fragment thereof, or pharmaceutical composition described herein for the manufacture of a medicament for blocking the entry of a betacoronavirus, such as a sarbecovirus, e.g., SARS-CoV-2 in a cell, such as an ACE2-expressing cell. The present disclosure provides the antibody or antigen-binding fragment thereof, of one more nucleic acids encoding the antibody antigen-binding fragment thereof, or pharmaceutical composition described herein, for use in blocking the entry of a betacoronavirus, such as a sarbecovirus, e.g., SARS-CoV-2 in a cell, such as an ACE2-expressing cell.

In another aspect, the disclosure provides a method of preventing or treating a disease or disorder caused by a severe acute respiratory syndrome coronavirus 2 (SARS-CoV-2) by administering to a person at risk of suffering from the disease or disorder or suffering from a disease or disorder caused by a severe acute respiratory syndrome coronavirus 2 (SARS-CoV-2), a therapeutically effective amount of the antibody or antigen-binding fragment thereof, of one more nucleic acids encoding the antibody antigen-binding fragment thereof, or pharmaceutical composition described herein. In some embodiments, the antibody or antigen-binding fragment thereof, of one more nucleic acids encoding the antibody antigen-binding fragment thereof, or pharmaceutical composition described herein can be administered individually. In some embodiments, the antibody or antigen-binding fragment thereof, of one more nucleic acids encoding the antibody antigen-binding fragment thereof, or pharmaceutical composition described herein can be administered in combination with one or more other antibodies, antigen-binding fragments or nucleic acids of the disclosure, e.g., as a cocktail comprising more than one antibodies, antibody fragments or nucleic acids. In some embodiments, the disease or disorder is designated COVID-19. The antibody or antigen-binding fragment thereof, of one more nucleic acids encoding the antibody antigen-binding fragment thereof, or pharmaceutical composition described herein (e.g., a monoclonal antibody) or a combination thereof can be administered at a dose sufficient to neutralize the SARS-CoV-2. In some embodiments, the method also includes administering an anti-viral drug, a viral entry inhibitor, or a viral attachment inhibitor. In some embodiments, the anti-viral drug is REMDESIVIR. In some embodiments, the antibody can be administered prior to or after exposure to SARS-CoV-2.

In an embodiment, the methods and uses defined herein are for the prevention, treatment and/or management of infections by the Wuhan original SARS-CoV-2 strain. In another embodiment, the methods and uses defined herein are for the prevention, treatment and/or management of infections by variants of the Wuhan original SARS-CoV-2 strain, such as the B.1.1.7 (also known as VOC-202012/01 or alpha (α)), 501Y.V2 (also known as B.1.351 or beta (β)), P.1 (also known as B.1.1.28.1 or gamma (γ)), B.1.617.2 (also known as delta (δ)), or B1.1.529 (Omicron) variant, as well as other variants of concern (VOC) such as B.1.429, B.1.526, B.1.525, and A.23.1 (see, e.g., www.cdc.gov/coronavirus/2019-ncov/cases-updates/variant-surveillance/variant-info.html). In an embodiment, the methods and uses defined herein are for the prevention, treatment and/or management of infections by the SARS-CoV-2 delta (6) variant. In an embodiment, the methods and uses defined herein are for the prevention, treatment and/or management of infections by the SARS-CoV-2 Omicron variant.

In an embodiment, the methods and uses defined herein comprises the administration or use of a mixture, combination or cocktail of antibodies or antigen-binding fragments thereof (or nucleic acids encoding same), such as the combination defined above.

The antibodies or antigen-binding fragments described herein may comprise one or more excipients to make the antibodies or antigen-binding fragments (or nucleic acids encoding same) suitable for nasal or oral administration. The antibodies or antigen-binding fragments thereof (or nucleic acids encoding same) described herein may comprise one or more excipients to make them suitable for oral administration (e.g., nebulization). Such formulations allow delivery of the antibodies or antigen-binding fragments (or nucleic acids encoding same) to specific sites of action along the nasopharyngeal, trachea, and/or lungs.

In certain embodiments, described herein, is a method of delivery of a composition comprising an antibody or antigen-binding fragment (or nucleic acids encoding same), or a mixture thereof, comprising any one or more of the CDRs or VH/VLs regions defined herein (FIGS. 7A-7K) to the respiratory system of an individual infected with SARS-CoV-2, the method comprising administering a nebulized formulation comprising an antibody or antigen-binding fragment (or nucleic acids encoding same), or a mixture thereof, comprising any one or more of the CDRs or VH/VLs regions defined herein (FIGS. 7A-7K) to the individual.

In certain embodiments, described herein, is the use of a nebulized antibody or antigen-binding fragment formulation comprising an antibody or antigen-binding fragment, or a mixture thereof, comprising any one or more of the CDRs or VH/VLs regions defined herein (FIGS. 7A-7K) for delivering the antibody or antigen-binding fragment, or mixture thereof, to the respiratory system of an individual infected with a betacoronavirus, such as a sarbecovirus, e.g., SARS-CoV-2.

In certain embodiments, described herein, is a method of treating a betacoronavirus, such as a sarbecovirus, e.g., SARS-CoV-2 infection in an individual comprising administering to the individual an antibody or antigen-binding fragment (or nucleic acids encoding same), or a mixture thereof, comprising any one or more of the CDRs or VH/VLs regions defined herein (FIGS. 7A-7K) to the respiratory system of the individual by nebulization.

In certain embodiments, described herein, is the use of a nebulized formulation comprising an antibody or antigen-binding fragment (or nucleic acids encoding same), or a mixture thereof, comprising any one or more of the CDRs or VH/VLs regions defined herein (FIGS. 7A-7K) for treating a betacoronavirus, such as a sarbecovirus, e.g., SARS-CoV-2 infection in an individual, wherein the formulation is for delivery of the antibody or antigen-binding fragment (or nucleic acids encoding same) to the respiratory system of the individual.

In certain embodiments, described herein, is a method of treating ARD associated with a betacoronavirus, such as a sarbecovirus, e.g., SARS-CoV-2 infection in an individual comprising administering to the individual an antibody or antigen-binding fragment (or nucleic acids encoding same), or a mixture thereof, comprising any one or more of the CDRs or VH/VLs regions defined herein (FIGS. 7A-7K) to the respiratory system of the individual by nebulization.

In certain embodiments, described herein, is the use of a nebulized formulation comprising an antibody or antigen-binding fragment (or nucleic acids encoding same), or a mixture thereof, comprising any one or more of the CDRs or VH/VLs regions defined herein (FIGS. 7A-7K) for treating ARD associated with a betacoronavirus, such as a sarbecovirus, e.g., SARS-CoV-2 infection in an individual, wherein the formulation is for delivery of the antibody or antigen-binding fragment (or nucleic acids encoding same) to the respiratory system of the individual.

Antibodies and antigen-binding fragments thereof (or nucleic acids encoding same) described herein may be nebulized using any suitable means such as a jet nebulizer (i.e., atomizer), a soft-mist inhaler, an ultrasonic wave nebulizer, or a vibrating mesh nebulizer.

For the prevention, treatment or reduction in the severity of a given disease or condition (viral disease such as COVID-19), the appropriate dosage of the antibody, antigen-binding fragment thereof (or nucleic acids encoding same), or pharmaceutical composition described herein will depend on the type of disease or condition to be treated, the severity and course of the disease or condition, whether the antibody, antigen-binding fragment thereof (or nucleic acids encoding same), or pharmaceutical composition is administered for preventive or therapeutic purposes, previous therapy, the patient's clinical history and response to the antibody, antigen-binding fragment thereof, or pharmaceutical composition, and the discretion of the attending physician. The antibody, antigen-binding fragment thereof (or nucleic acids encoding same), or pharmaceutical composition described herein may be suitably administered to the patient at one time or over a series of treatments. Preferably, it is desirable to determine the dose-response curve in vitro, and then in useful animal models prior to testing in humans. The present disclosure provides dosages for the antibody or antigen-binding fragment thereof (or nucleic acids encoding same), or pharmaceutical composition. For example, depending on the type and severity of the disease, about 1 μg/kg to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

In an embodiment, the dosage of the anti-SARS-CoV-2 antibodies or antigen-binding fragments thereof, or combination thereof, is from 1 mg to 30 mg. In a further embodiment, the dosage of the anti-SARS-CoV-2 antibodies or antigen-binding fragments thereof, or combination thereof, is from 2 mg to 20 mg. In a further embodiment, the dosage of the anti-SARS-CoV-2 antibodies or antigen-binding fragments thereof, or combination thereof, is from 5 mg to 15 mg. In a further embodiment, the dosage of the anti-SARS-CoV-2 antibodies or antigen-binding fragments thereof, or combination thereof, is about 6, 7, 8, 9, 10, 11, 12, 13 or 14 mg. In a further embodiment, the dosage of the anti-SARS-CoV-2 antibodies or antigen-binding fragments thereof, or combination thereof, is about 10 mg.

As used herein the term “treating” or “treatment” in reference to viral infection or disease is meant to refer to administration of the agent after infection that leads to a reduction/improvement in one or more symptoms or pathological features associated with said viral disease (e.g., COVID-19). Non-limiting examples include a decrease in viral load, reduction of cough, fever, fatigue, shortness of breath, reduction/prevention of acute respiratory distress syndrome (ARDS), reduction/prevention of multi-organ failure, septic shock, and blood clots, hospitalization, etc.

As used herein the term “preventing” or “prevention” in reference to viral infection or disease is meant to refer to administration of the agent prior to infection that leads to protection from being infected or from developing the viral disease (e.g., COVID-19), to a delay in the development of the disease, or to a reduction of one or more symptoms or pathological features associated with the viral disease.

In an embodiment, the administration/use of the antibody, antigen-binding fragment thereof (or nucleic acids encoding same), or pharmaceutical composition described herein delays the onset of one or more symptoms of a betacoronavirus or sarbecovirus-caused infection, e.g., SARS-CoV-2-caused infection (e.g., COVID-19).

The antibody, antigen-binding fragment thereof (or nucleic acids encoding same), or pharmaceutical composition described herein may be used alone or in combination with other prophylactic agents such as antivirals, anti-inflammatory agents, vaccines, immunotherapies, etc. The combination of active agents and/or compositions comprising same may be administered or co-administered (e.g., consecutively, simultaneously, at different times) in any conventional dosage form. Co-administration in the context of the present disclosure refers to the administration of more than one therapeutic in the course of a coordinated treatment to achieve an improved clinical outcome. Such co-administration may also be coextensive, that is, occurring during overlapping periods of time. For example, a first agent (e.g., the antibody or antigen-binding fragment thereof, liposomes, pharmaceutical composition or vaccine described herein) may be administered to a patient before, concomitantly, before and after, or after a second active agent (e.g., an antiviral or anti-inflammatory agent) is administered. The agents may in an embodiment be combined/formulated in a single composition and thus administered at the same time. In another embodiment, the antibody or antigen-binding fragment thereof described herein is used in combination with one or more additional anti-SARS-CoV-2 antibodies or antigen-binding fragments thereof (or nucleic acids encoding same). In a further embodiment, the antibody or antigen-binding fragment thereof (or nucleic acids encoding same) described herein and the one or more additional anti-SARS-CoV-2 antibodies are present in the same composition, e.g., in an antibody cocktail.

In an embodiment, the antibody or antigen-binding fragment thereof (or nucleic acids encoding same) is for administration prior to exposure to a betacoronavirus, such as a sarbecovirus, e.g., SARS-CoV-2. In another embodiment, the antibody is for administration after exposure to a betacoronavirus, such as a sarbecovirus, e.g., SARS-CoV-2. In another embodiment, the antibody is for administration prior to and after exposure to a betacoronavirus, such as a sarbecovirus, e.g., SARS-CoV-2.

In an embodiment, the antibody or antigen-binding fragment thereof (or nucleic acids encoding same) is for administration prior to development of the viral disease (e.g., COVID-19). In another embodiment, the antibody or antigen-binding fragment thereof (or nucleic acids encoding same) is for administration after development of the viral disease (e.g., COVID-19). In another embodiment, the antibody or antigen-binding fragment thereof (or nucleic acids encoding same) is for administration prior to and after development of the viral disease (e.g., COVID-19).

In another aspect, provided herein is a method of detecting the presence of a betacoronavirus, such as a sarbecovirus, e.g., SARS-CoV-2, in a sample by contacting the sample with an antibody or antigen-binding fragment thereof of the disclosure, and detecting the presence or absence of an antibody-antigen complex, thereby detecting the presence of the betacoronavirus, such as the sarbecovirus, e.g., SARS-CoV-2, in a sample. Any suitable sample can be used in the methods of the disclosure. In some embodiments, the sample can be obtained from blood, cheek scraping or swab, nasal swab, saliva, biopsy, urine, feces, sputum, nasal aspiration, or semen. In some embodiments, the sample is obtained from blood. In some embodiments, the sample is saliva, blood, plasma, or serum. In some embodiments, the sample can be a sample collected from a surface suspected of being contaminated with a betacoronavirus, such as a sarbecovirus, e.g., SARS-CoV-2. In an embodiment, the antibody or antigen-binding fragment thereof is bound to a detectable label such as a fluorophore, a radioactive label, a colloidal gold particle, a magnetic particle, a quantum dot, etc.

In another aspect, provided herein is a method of delaying the onset of one or more symptoms of a betacoronavirus-, such as a sarbecovirus-, e.g., SARS-CoV-2-caused infection (e.g., Severe Acute Respiratory Syndrome such as COVID-19), comprising administering to a person at risk of suffering from such infection a therapeutically effective amount of an antibody or antigen-binding fragment thereof disclosed herein.

In another aspect, the disclosure provides an antigen-binding composition comprising a monoclonal antibody or antigen-binding fragment thereof specific to an epitope in the viral envelope spike protein (S2P) of a Severe Acute Respiratory Syndrome Coronavirus 2 (SARS-CoV-2), or one or more nucleic acids encoding said monoclonal antibody or antigen-binding fragment thereof, wherein the neutralizing antibody or antigen-binding fragment thereof comprises a heavy chain comprising amino acid sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61 and 63 and a light chain comprising amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62 and 64.

In an embodiment, the subject or patient has a weakened immune system and a reduced ability to fight viral infections such as SARS-CoV-2 infection. In another embodiment, the subject or patient is an immunosuppressed or immunocompromised subject or patient. Immunosuppression may be caused by certain diseases or conditions, such as AIDS, cancer, diabetes, malnutrition, and certain genetic disorders, or certain drugs or treatments such as anticancer drugs, radiation therapy, and stem cell or organ transplant. In an embodiment, the subject or patient is an elderly subject or patient, for example a subject or patient having 60 years old or more, 65 years old or more, 70 years old or more, 75 years old or more, or 80 years old or more, who typically develop a weaker immune response to vaccines and infections.

In another aspect, the disclosure provides kits comprising one or more antibodies or antigen-binding fragments thereof, or nucleic acids encoding said antibodies or antigen-binding fragments thereof, of the disclosure. Kits include one or more containers comprising by way of example, and not limitation, one or more antibodies or antigen-binding fragments thereof, or nucleic acids encoding said antibodies or antigen-binding fragments thereof, specific to severe acute respiratory syndrome coronavirus 2 (SARS-CoV-2) or combinations thereof and instructions for use in accordance with any of the methods of the disclosure. In some embodiments of the kits, the antibodies or antigen-binding fragments thereof are bound to a detectable label. Any suitable detectable label can be used, such as a fluorophore, a radioactive label, a colloidal gold particle, a magnetic particle, a quantum dot, etc.

Generally, instructions comprise a description of administration or instructions for performance of an assay. The containers can be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The kits are provided in suitable packaging. Suitable packaging includes, but is not limited to, vials, bottles, jars, flexible packaging (e.g., sealed Mylar or plastic bags), and the like. A kit can have a sterile access port (e.g., the container can be an intravenous solution bag or a vial having a stopper pierceable by a hypodermic injection needle). The container can also have a sterile access port (e.g., the container can be an intravenous solution bag or a vial having a stopper pierceable by a hypodermic injection needle). Kits can optionally provide additional components such as buffers and interpretive information. Normally, the kit comprises a container and a label or package insert(s) on or associated with the container. In an embodiment, the kit includes a device for intrapulmonary administration of the the antibody or antigen-binding fragment thereof (or nucleic acids encoding same), such as a nebulizer, metered-dose inhaler, or dry powder inhaler.

The following examples are provided to further illustrate but not limit the disclosure.

### EXAMPLES

### Example 1: Characterization of the Antibody Responses to SARS-CoV-2 Infection

The present inventors performed an in-depth characterization of the antibody responses to SARS-CoV-2 infection, including the clonality of the B cell responses. This work reveals whether and how the development of neutralizing antibody responses is linked with the severity of clinical symptoms and with the control of infection.

Human subject characteristics and available biospecimens. B-cells and plasma were obtained from SARS-CoV-2-infected subjects from Montreal, QC, Canada after positive diagnosis. Biospecimens were collected as follows: blood is collected in ACD tubes, processed and plasma and viable peripheral blood mononuclear cells (PBMCs) are isolated and cryopreserved for future analysis. This study is IRB approved and all participants signed informed consent for use of biospecimens in biomedical research.

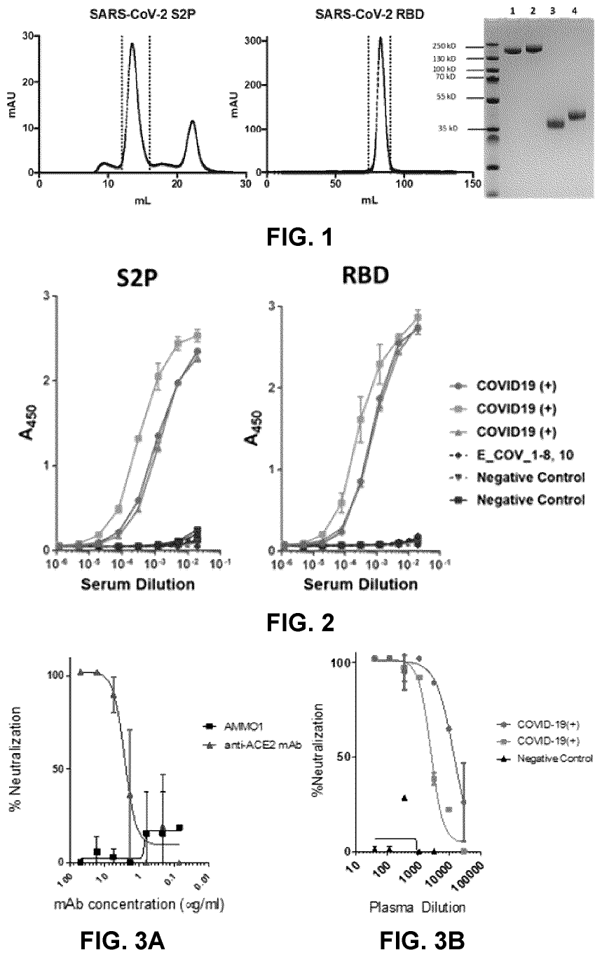

Expression and Purification of SARS-CoV-2 envelope glycoproteins. Coronavirus surface envelope glycoproteins are targets of neutralizing antibodies (5-8). The SARS-CoV-2 envelope glycoprotein is a surface-exposed class-I fusion protein that, similarly to other coronaviruses, comprises an ectodomain, a transmembrane region and a short intracellular domain. The ectodomain consists of two non-covalently associated subunits: a receptor binding domain subunit (S1) and a membrane-fusion subunit (S2). High resolution structures of SARS-CoV-2 envelope glycoproteins have been recently published (9, 10). Vectors and expression platforms were used to produce large quantities of two different versions of the viral glycoprotein in 293 cells. The inventors possess plasmids to express two forms of the SARS-CoV-2 envelope: S2P and receptor binding domain (RBD). The S2P represents the entire ectodomain and encodes residues 1-1208 (GenBank:MN908947) (9, 11). The RBD form encodes only residues 319-591 of SARS-CoV-2 S and is cloned upstream of a monomeric Fc separated by HRV3C protease cleavage site (9). The vectors were obtained from Dr. McLellan who published the first atomic level structure of a stabilized prefusion SARS-CoV-2 Spike glycoproteins (9). The two plasmids were transfected in 293 cells and the expressed proteins were purified using affinity purification followed by size exclusion chromatography (SEC) (FIG. 1). 2 mgs of S2P and 12 mgs of RBD were purified and employed for serological evaluation and B cell isolation (see below).

Serological analysis. The two purified SARS-CoV-2 S proteins (S2P and RBD) were used in an ELISA assay with three SARS-CoV-2 seropositive serum samples (COVID19 (+)), two seronegative control sera (Negative Control) and sera from nine subjects infected with endemic CoV viruses in the Seattle metropolitan area (E_COV_1-8, 10) (FIG. 2). The sera from the three SARS-CoV-2 infected patients showed reactivity to both the S2P and the RBD. Neither the endemic CoV sera nor the negative control sera displayed reactivity to either SARS-CoV-2 proteins. These results indicate that the inventors have reagents that specifically identify the presence of serum antibodies against the Env of SARS-CoV-2.

Neutralization assays. A pseudoviral neutralization assay was employed to assess the neutralizing activities of polyclonal sera and of mAbs isolated from S2P- and RBD-specific B cells. Pseudoviral particles in which the full-length S protein of SARS-CoV-2 pseudotyped on an HIV backbone delivers a luciferase reporter gene to target cells (293 cells expressing ACE2) upon entry were produced. Consistent with a previous report, pseudoviral infection was inhibited by anti-ACE2 antibodies (12), but not a control anti-EBV control mAb (FIG. 3A) (13). Plasma from donors with confirmed COVID-19 infection also neutralized SARS CoV-2 pseudovirus whereas control plasma from an uninfected donor did not (FIG. 3B).

### Example 2: Isolation of Potent Neutralizing Monoclonal Antibodies from COVID-19-Infected Patients

To identify the epitopes targeted by SARS-CoV-2 neutralizing antibodies, S2P- and RBD-specific B cells from the PBMCs of patients (once serum neutralizing activities were confirmed) were isolated. Using methodologies previously described (13, 16, 17), the inventors sequenced the VH/VL genes from individual B cells and expressed them as monoclonal antibodies (mAbs). Their binding (S2P, RBD- or dual-specific) affinities and neutralizing potencies are then determined.

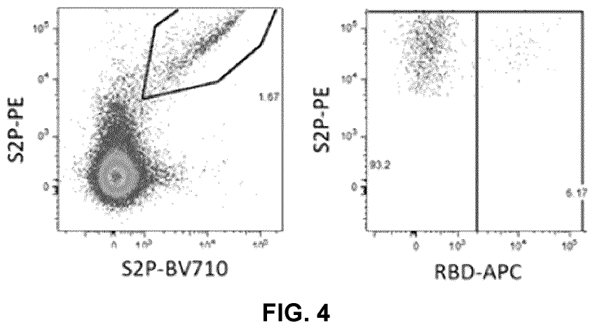

Isolation of antigen-specific B cells. Memory B cells from one SARS-CoV-2 patient (approximately 3 weeks post infection) were stained with S2P and RBD and single-cell sorted using standard B cell isolation protocols (FIG. 4). IgG+ B cells were specifically sorted, first by staining the cells with S2P separately labeled with two different fluorophores and selecting the double positive cells (see gate, left panel). Within this population, the cells that were specific for RBD, which was labeled with a different fluorophore (right panel), were identified. About 6% of the IgG+ B cells bound both RBD and S2P. In total, 768 envelope-specific B cells were isolated, and the sequencing of their VH/VL genes was completed. Incomplete VH/VL sequences were removed and VH/VL pairs were identified. Each of them was expressed as IgG and tested for binding and neutralization.

MAb-binding. The binding of several mAbs isolated from COVID-19-infected patients to SARS-CoV2 S2P, SARS-CoV2 RBD and SARS-CoV1 S2P was assessed using the strategy in FIG. 4. COVID-19-derived mAbs were loaded onto an anti-human Fc probe and dipped in the SARS-CoV2 recombinant envelope proteins to measure binding using biolayer interferometry (BLI): RBD (receptor binding domain) and S2P (extracellular portion of the trimeric envelope spike). Of the mAbs tested, several bound to S2P. The binding results are shown in FIGS. 5A-E. Several mAbs (identified as Mab #1-Mab #9, Mab #11-Mab #13, Mab #17, Mab #20-Mab #23, Mab #25, Mab #27, Mab #35-Mab #39, and Mab #41-Mab #48) have been tested.

Neutralizing activity of exemplary anti-CoV2 monoclonal antibodies. HIV-1 derived lentiviral particles pseudotyped with the SARS CoV-2 Spike (S) protein capable of delivering a luciferase reporter gene were mixed with the indicated monoclonal antibodies (Mab #1, Mab #7, Mab #25 and Mab #43), incubated for 1 hour and then added to cells stably expressing ACE2; the SARS CoV-2 receptor. The ACE2 ectodomain fused to an IgG1 Fc (ACE2-Fc), which acts as a competitive inhibitor of spike-binding to cell-surface ACE2, and the anti-EBV gH/gL antibody AMMO1 were included as positive and negative controls, respectively. 48 hours later the cells were lysed, and luciferase activity was measured. Neutralizing activity is reported as the reduction in infectivity in the presence of the mAb relative to the infectivity in the absence of mAb. The viral particles and the cell line were generated as described in Crawford et al. (Viruses 2020, 12(5), 513). The results are shown in FIG. 6.

### Example 3: Further Characterization of Neutralizing Antibodies Mab #1 and Mab #25

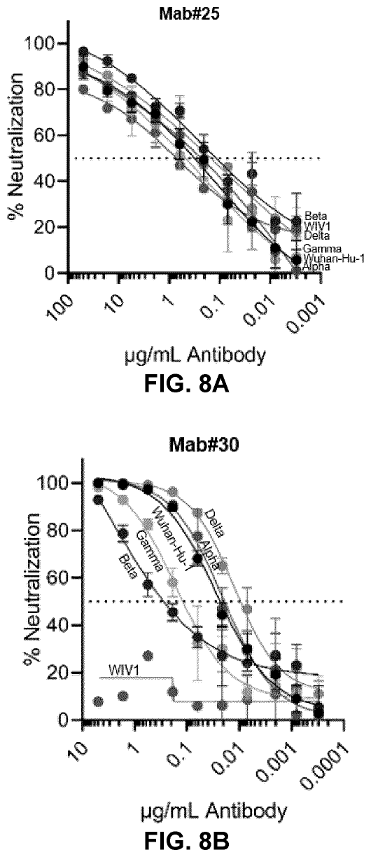

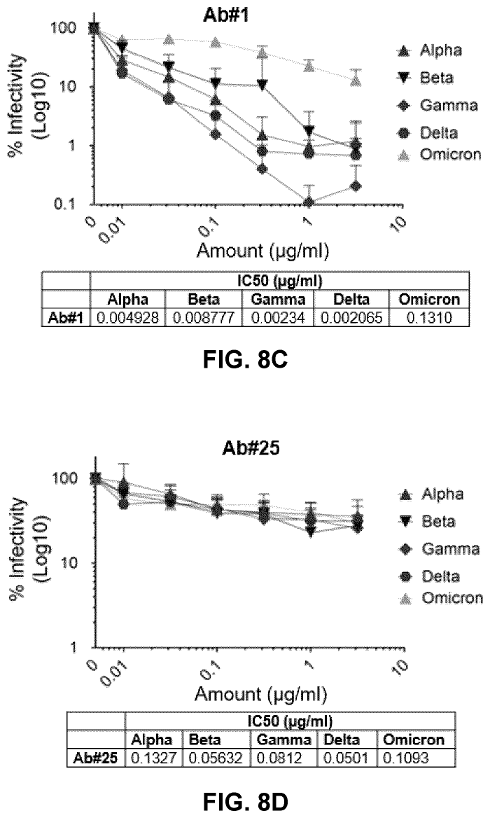

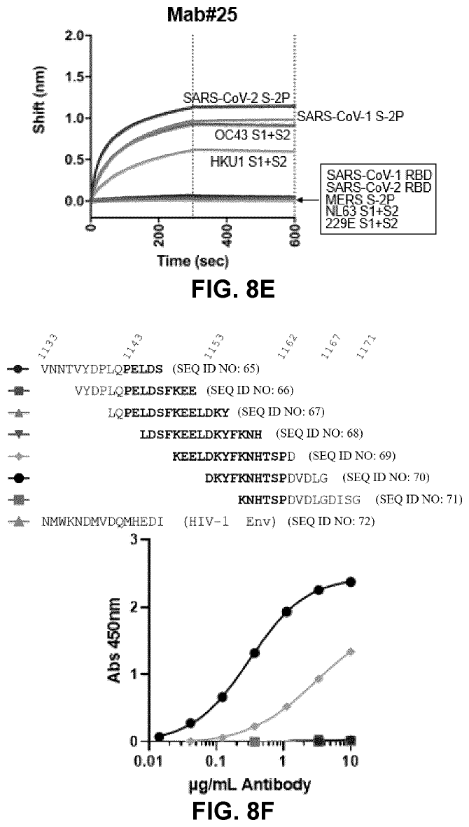

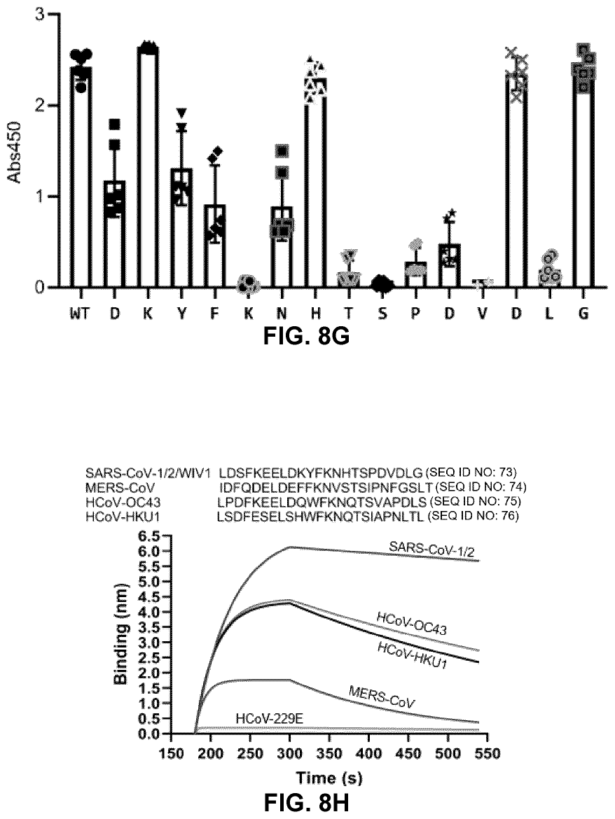

The ability of Mab #25 to neutralize additional SARS-CoV-2 variants of concern (VOCs) Alpha (B.1.1.7), Delta (B.1.617.2) and Gamma (P.1) and a more distantly related SARS-like bat coronavirus, WIV1, which uses ACE2 as an entry receptor and can infect human cell lines (thus representing a bat CoV with pandemic potential), was assessed. Mab #25 neutralized all variants and WIV1 with comparable potency (FIG. 8A). In contrast, an RBD-directed mAb (Mab #30) showed reduced potency against the Beta and Gamma VOCs, both of which harbor mutations in the RBD at position 417 (FIG. 8B), and no neutralizing activity against WIV1. The anti-EBV gH/gL antibody AMMO1 (negative control) had no neutralizing activity. Cell staining experiments revealed that Mab #1 and Mab #25 have the ability to bind to the Spike protein from several SARS-CoV-2 VOCs including D614G mutant, B.1.1.7, B.1.1.7 E484K, B.1.351, P.1, B.1.429, B.1.526, B.1.617.2 and B.1.617.2, and infectivity assays using pseudoviruses showed that these two antibodies are able to neutralize the wild-type Wuhan-Hu-1 strain as well as the D614G, B.1.1.7, B.1.351, P.1, B.1.526 and B.1.617.2 variants, with IC₅₀ between ˜0.004 and ˜0.014 μg/ml for Mab #1 and between ˜0.05 to ˜0.2 μg/ml for Mab #25. Additional neutralization experiments were performed using Mab #1 and Mab #25 on pseudoviruses expressing Spike proteins from various VOCs including the Omicron variant that comprises several mutations. As shown in FIGS. 8C and 8D, Mab #1 maintain the ability to neutralize the various variants, although with reduced potency for some of the variants, notably Omicron, whereas Mab #25 was able to neutralize the different variants (including Omicron) with similar potency.

The results depicted in FIG. 8E show that Mab #25 is able to also bind to the S2 domain of the S protein from SARS-CoV-1 and from the two endemic human beta coronaviruses that causes the common cold, OC43 and HKU1. An ELISA using overlapping 15mer linear peptides spanning the stem helix region (1143-1162) from SARS-CoV-2 revealed that Mab #25 is able to bind to two peptides encompassing amino acids 1149-1163 and 1153-1167, with stronger binding to the latter, but not to any of the other SARS-CoV-2 peptides or to a control peptide from HIV-1 Env (FIG. 8F). Alanine scanning of a stem helix peptide was conducted to assess the relative contributions of the various residues to the binding to Mab #25. As shown in FIG. 8G, mutating Lys₁₁₅₇, T₁₁₆₀, S₁₁₆₁, P₁₁₆₂, D₁₁₆₃, V₁₁₆₄, or Leu₁₁₆₆ led to a significant reduction of Mab #25 binding. Since several of the Mab #25 contact residues are conserved in beta coronaviruses, the ability of Mab #25 to bind peptides derived from additional beta coronaviruses spanning the stem helix region was tested by ELISA. Mab #25 was shown to bind equally well to peptides derived from SARS-CoV-1/2/WIV1, MERS-CoV, and HCoV-OC43, and to a slightly weaker extent to a peptide derived from HCoV-HKU1 (FIG. 8H). However, despite binding to the stem helix peptide from MERS-CoV, HCoV-OC43 and HCoV-HKU1, Mab #25 did not recognize cell-surface expressed spikes from MERS-CoV, HCoV-OC43 and HCoV-HKU, and failed to neutralize a MERS-CoV pseudovirus or authentic HCoV-OC43, suggesting that the epitope is present but not equally accessible in the native conformation of the spike protein among the various coronaviruses. These results indicate that Mab #25 binds to a linear epitope on S2 that is conserved among beta coronaviruses and is able to neutralize sarbecoviruses (SARS-CoV-1, SARS-CoV-2, WIV1) and the binding is unaffected by mutations found in several SARS-CoV-2 variants.

Mutagenesis experiments showed that Mab #1 bind to the tip region (485-GFN-487 loop) within the receptor-binding motif (RBM) of S1 as mutations in any of residues G₄₈₅, F₄₈₆ or N₄₈₇ affected Mab #1 binding. Since mutations within the 485-GFN-487 loop also impair ACE2 binding, escape mutations at these positions would likely result in a high fitness cost for the virus.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Wu Y, Ho W, Huang Y, Jin D Y, Li S, Liu S L, Liu X, Qiu J, Sang Y, Wang Q, Yuen K Y, Zheng Z M. SARS-CoV-2 is an appropriate name for the new coronavirus. Lancet. 2020. Epub 2020/03/11. doi: 10.1016/S0140-6736(20)30557-2. PubMed PMID: 32151324.
2. Mahase E. Covid-19: WHO declares pandemic because of “alarming levels” of spread, severity, and inaction. BMJ. 2020; 368:m1036. Epub 2020/03/14. doi: 10.1136/bmj.m1036. PubMed PMID: 32165426.
3. Dong E, Du H, Gardner L. An interactive web-based dashboard to track COVID-19 in real time. Lancet Infect Dis. 2020. Epub 2020/02/23. doi: 10.1016/S1473-3099(20)30120-1. PubMed PMID: 32087114.
4. Zhou P, Yang X L, Wang X G, Hu B, Zhang L, Zhang W, Si H R, Zhu Y, Li B, Huang C L, Chen H D, Chen J, Luo Y, Guo H, Jiang R D, Liu M Q, Chen Y, Shen X R, Wang X, Zheng X S, Zhao K, Chen Q J, Deng F, Liu L L, Yan B, Zhan F X, Wang Y Y, Xiao G F, Shi Z L. A pneumonia outbreak associated with a new coronavirus of probable bat origin. Nature. 2020; 579(7798):270-3. Epub 2020/02/06. doi: 10.1038/s41586-020-2012-7. PubMed PMID: 32015507.
5. Zhang H, Wang G, Li J, Nie Y, Shi X, Lian G, Wang W, Yin X, Zhao Y, Qu X, Ding M, Deng H. Identification of an antigenic determinant on the S2 domain of the severe acute respiratory syndrome coronavirus spike glycoprotein capable of inducing neutralizing antibodies. J Virol. 2004; 78(13):6938-45. Epub 2004/06/15. doi: 10.1128/JVI.78.13.6938-6945.2004.
PubMed PMID: 15194770; PMCID: PMC421668.
6. Rockx B, Corti D, Donaldson E, Sheahan T, Stadler K, Lanzavecchia A, Baric R. Structural basis for potent cross-neutralizing human monoclonal antibody protection against lethal human and zoonotic severe acute respiratory syndrome coronavirus challenge. J Virol. 2008; 82(7):3220-35. Epub 2008/01/18. doi: 10.1128/JVI.02377-07. PubMed PMID: 18199635; PMCID: PMC2268459.
7. Wang L, Shi W, Chappell J D, Joyce M G, Zhang Y, Kanekiyo M, Becker M M, van Doremalen N, Fischer R, Wang N, Corbett K S, Choe M, Mason R D, Van Galen J G, Zhou T, Saunders K O, Tatti K M, Haynes L M, Kwong P D, Modjarrad K, Kong W P, McLellan J S, Denison M R, Munster V J, Mascola J R, Graham B S. Importance of Neutralizing Monoclonal Antibodies Targeting Multiple Antigenic Sites on the Middle East Respiratory Syndrome Coronavirus Spike Glycoprotein To Avoid Neutralization Escape. J Virol. 2018; 92(10). Epub 2018/03/09. doi: 10.1128/JVI.02002-17. PubMed PMID: 29514901; PMCID: PMC5923077.
8. Walls A C, Xiong X, Park Y J, Tortorici M A, Snijder J, Quispe J, Cameroni E, Gopal R, Dai M, Lanzavecchia A, Zambon M, Rey F A, Corti D, Veesler D. Unexpected Receptor Functional Mimicry Elucidates Activation of Coronavirus Fusion. Cell. 2019; 176(5):1026-39 e15. Epub 2019/02/05. doi: 10.1016/j.cell.2018.12.028. PubMed PMID: 30712865; PMCID: PMC6751136.
9. Wrapp D, Wang N, Corbett K S, Goldsmith J A, Hsieh C L, Abiona O, Graham B S, McLellan J S. Cryo-E M structure of the 2019-nCoV spike in the prefusion conformation. Science. 2020; 367(6483):1260-3. Epub 2020/02/23. doi: 10.1126/science.abb2507. PubMed PMID: 32075877.
10. Walls A C, Park Y J, Tortorici M A, Wall A, McGuire A T, Veesler D. Structure, Function, and Antigenicity of the SARS-CoV-2 Spike Glycoprotein. Cell. 2020. Epub 2020/03/11. doi: 10.1016/j.cell.2020.02.058. PubMed PMID: 32155444.
11. Pallesen J, Wang N, Corbett K S, Wrapp D, Kirchdoerfer R N, Turner H L, Cottrell C A, Becker M M, Wang L, Shi W, Kong W P, Andres E L, Kettenbach A N, Denison M R, Chappell J D, Graham B S, Ward A B, McLellan J S. Immunogenicity and structures of a rationally designed prefusion MERS-CoV spike antigen. Proc Natl Acad Sci USA. 2017; 114(35):E7348-E57. Epub 2017/08/16. doi: 10.1073/pnas.1707304114. PubMed PMID: 28807998; PMCID: PMC5584442.
12. Hoffmann M, Kleine-Weber H, Schroeder S, Kruger N, Herrier T, Erichsen S, Schiergens T S, Herrier G, Wu N H, Nitsche A, Muller M A, Drosten C, Pohlmann S. SARS-CoV-2 Cell Entry Depends on ACE2 and TMPRSS2 and Is Blocked by a Clinically Proven Protease Inhibitor. Cell. 2020. Epub 2020/03/07. doi: 10.1016/j.cell.2020.02.052. PubMed PMID: 32142651.
13. Snijder J, Ortego M S, Weidle C, Stuart A B, Gray M D, McElrath M J, Pancera M, Veesler D, McGuire A T. An Antibody Targeting the Fusion Machinery Neutralizes Dual-Tropic Infection and Defines a Site of Vulnerability on Epstein-Barr Virus. Immunity. 2018; 48(4):799-811 e9. Epub 2018/04/19. doi: 10.1016/j.immuni.2018.03.026. PubMed PMID: 29669253; PMCID: PMC5909843.
14. Fatima Amanat T N, Veronika Chromikova, Shirin Strohmeier, Daniel Stadlbauer, Andres Javier, Kaijun Jiang, Guha Asthagiri-Arunkumar, Jose Polanco, Maria Bermudez-Gonzalez, Daniel Caplivski, Allen Cheng, Katherine Kedzierska, Olli Vapalahti, Jussi Hepojoki, Viviana Simon, Florian Krammer. A serological assay to detect SARS-CoV-2 seroconversion in humans. medRXiv. 2020. doi: https://doi.org/10.1101/2020.03.17.20037713.
15. Roque-Barreira M C, Campos-Neto A. Jacalin: an IgA-binding lectin. J Immunol. 1985; 134(3):1740-3. Epub 1985/03/01. PubMed PMID: 3871459.
16. Parks K R, MacCamy A J, Trichka J, Gray M, Weidle C, Borst A J, Khechaduri A, Takushi B, Agrawal P, Guenaga J, Wyatt R T, Coler R, Seaman M, LaBranche C, Montefiori D C, Veesler D, Pancera M, McGuire A, Stamatatos L. Overcoming Steric Restrictions of VRC01 HIV-1 Neutralizing Antibodies through Immunization. Cell reports. 2019; 29(10):3060-72 e7. Epub 2019/12/05. doi: 10.1016/j.celrep.2019.10.071. PubMed PMID: 31801073; PMCID: PMC6936959.
17. Dosenovic P, Pettersson A K, Wall A, Thientosapol E S, Feng J, Weidle C, Bhullar K, Kara E E, Hartweger H, Pai J A, Gray M D, Parks K R, Taylor J J, Pancera M, Stamatatos L, Nussenzweig M C, McGuire A T. Anti-idiotypic antibodies elicit anti-HIV-1-specific B cell responses. J Exp Med. 2019; 216(10):2316-30. Epub 2019/07/28. doi: 10.1084/jem.20190446. PubMed PMID: 31345931; PMCID: PMC6780999.
18. Zhou T, Zhu J, Wu X, Moquin S, Zhang B, Acharya P, Georgiev I S, Altae-Tran H R, Chuang G Y, Joyce M G, Do Kwon Y, Longo N S, Louder M K, Luongo T, McKee K, Schramm C A, Skinner J, Yang Y, Yang Z, Zhang Z, Zheng A, Bonsignori M, Haynes B F, Scheid J F, Nussenzweig M C, Simek M, Burton D R, Koff W C, Program N C S, Mullikin J C, Connors M, Shapiro L, Nabel G J, Mascola J R, Kwong P D. Multidonor analysis reveals structural elements, genetic determinants, and maturation pathway for HIV-1 neutralization by VRC01-class antibodies. Immunity. 2013; 39(2):245-58. doi: 10.1016/j.immuni.2013.04.012. PubMed PMID: 23911655; PMCID: 3985390.
19. Prabakaran P, Gan J, Feng Y, Zhu Z, Choudhry V, Xiao X, Ji X, Dimitrov D S. Structure of severe acute respiratory syndrome coronavirus receptor-binding domain complexed with neutralizing antibody. J Biol Chem. 2006; 281(23):15829-36. Epub 2006/04/07. doi: M600697200 [pii]10.1074/jbc.M600697200. PubMed PMID: 16597622.
20. Yuan M, Wu, N. C., Zhu, X., Lee, C-C. D., So, R. T. Y, Lv, H., Mok, C. K. P., Wilson, I. A. A highly conserved cryptic epitope in the receptor-binding domains of SARS-CoV-2 and SARS-CoV. BioRxiv. 2020. doi: https://doi.org/10.1101/2020.03.13.991570. T.
21. Pancera M, Zhou T, Druz A, Georgiev I S, Soto C, Gorman J, Huang J, Acharya P, Chuang G Y, Ofek G, Stewart-Jones G B, Stuckey J, Bailer R T, Joyce M G, Louder M K, Tumba N, Yang Y, Zhang B, Cohen M S, Haynes B F, Mascola J R, Morris L, Munro J B, Blanchard S C, Mothes W, Connors M, Kwong P D. Structure and immune recognition of trimeric pre-fusion HIV-1 Env. Nature. 2014; 514(7523):455-61. Epub 2014/10/09. doi: 10.1038/nature13808. PubMed PMID: 25296255; PMCID: 4348022.
22. Kisalu N K, Idris A H, Weidle C, Flores-Garcia Y, Flynn B J, Sack B K, Murphy S, Schon A, Freire E, Francica J R, Miller A B, Gregory J, March S, Liao H X, Haynes B F, Wiehe K, Trama A M, Saunders K O, Gladden M A, Monroe A, Bonsignori M, Kanekiyo M, Wheatley A K, McDermott A B, Farney S K, Chuang G Y, Zhang B, Kc N, Chakravarty S, Kwong P D, Sinnis P, Bhatia S N, Kappe S H I, Sim B K L, Hoffman S L, Zavala F, Pancera M, Seder R A. A human monoclonal antibody prevents malaria infection by targeting a new site of vulnerability on the parasite. Nat Med. 2018; 24(4):408-16. Epub 2018/03/20. doi: 10.1038/nm.4512. PubMed PMID: 29554083; PMCID: PMC5893371.
23. Dubrovskaya V, Tran K, Ozorowski G, Guenaga J, Wilson R, Bale S, Cottrell C A, Turner H L, Seabright G, O'Dell S, Torres J L, Yang L, Feng Y, Leaman D P, Vazquez Bernat N, Liban T, Louder M, McKee K, Bailer R T, Movsesyan A, Doria-Rose N A, Pancera M, Karlsson Hedestam G B, Zwick M B, Crispin M, Mascola J R, Ward A B, Wyatt R T. Vaccination with Glycan-Modified HIV NFL Envelope Trimer-Liposomes Elicits Broadly Neutralizing Antibodies to Multiple Sites of Vulnerability. Immunity. 2019; 51(5):915-29 e7. Epub 2019/11/17. doi: 10.1016/j.immuni.2019.10.008. PubMed PMID: 31732167; PMCID: PMC6891888.
24. Bianchi M, Turner H L, Nogal B, Cottrell C A, Oyen D, Pauthner M, Bastidas R, Nedellec R, McCoy L E, Wilson I A, Burton D R, Ward A B, Hangartner L. Electron-Microscopy-Based Epitope Mapping Defines Specificities of Polyclonal Antibodies Elicited during HIV-1 BG505 Envelope Trimer Immunization. Immunity. 2018; 49(2):288-300 e8. Epub 2018/08/12. doi: 10.1016/j.immuni.2018.07.009. PubMed PMID: 30097292; PMCID: PMC6104742.
25. Chen L, Kwon Y D, Zhou T, Wu X, O'Dell S, Cavacini L, Hessell A J, Pancera M, Tang M, Xu L, Yang Z Y, Zhang M Y, Arthos J, Burton D R, Dimitrov D S, Nabel G J, Posner M R, Sodroski J, Wyatt R, Mascola J R, Kwong P D. Structural basis of immune evasion at the site of CD4 attachment on HIV-1 gp120. Science. 2009; 326(5956):1123-7. Epub 2009/12/08. doi: 326/5956/1123 [pii] 10.1126/science.1175868. PubMed PMID: 19965434.
26. Li F, Li W, Farzan M, Harrison S C. Structure of SARS coronavirus spike receptor-binding domain complexed with receptor. Science. 2005; 309(5742):1864-8. Epub 2005/09/17. doi: 309/5742/1864 [pii] 10.1126/science.1116480. PubMed PMID: 16166518.
27. Song W, Gui M, Wang X, Xiang Y. Cryo-E M structure of the SARS coronavirus spike glycoprotein in complex with its host cell receptor ACE2. PLoS Pathog. 2018; 14(8):e1007236. Epub 2018/08/14. doi: 10.1371/journal.ppat.1007236. PubMed PMID: 30102747; PMCID: PMC6107290.
28. Yan R, Zhang Y, Li Y, Xia L, Guo Y, Zhou Q. Structural basis for the recognition of SARS-CoV-2 by full-length human ACE2. Science. 2020; 367(6485):1444-8. Epub 2020/03/07. doi: 10.1126/science.abb2762. PubMed PMID: 32132184.
29. Wang Q, Zhang Y, Wu L, Niu S, Song C, Zhang Z, Lu G, Qiao C, Hu Y, Yuen K Y, Wang Q, Zhou H, Yan J, Qi J. Structural and Functional Basis of SARS-CoV-2 Entry by Using Human ACE2. Cell. 2020. Epub 2020/04/11. doi: 10.1016/j.cell.2020.03.045. PubMed PMID: 32275855; PMCID: PMC7144619.
30. McLellan J S, Chen M, Joyce M G, Sastry M, Stewart-Jones G B, Yang Y, Zhang B, Chen L, Srivatsan S, Zheng A, Zhou T, Graepel K W, Kumar A, Moin S, Boyington J C, Chuang G Y, Soto C, Baxa U, Bakker A Q, Spits H, Beaumont T, Zheng Z, Xia N, Ko S Y, Todd J P, Rao S, Graham B S, Kwong P D. Structure-based design of a fusion glycoprotein vaccine for respiratory syncytial virus. Science. 2013; 342(6158):592-8. doi: 10.1126/science.1243283. PubMed PMID: 24179220; PMCID: 4461862.
31. McLellan J S, Chen M, Leung S, Graepel K W, Du X, Yang Y, Zhou T, Baxa U, Yasuda E, Beaumont T, Kumar A, Modjarrad K, Zheng Z, Zhao M, Xia N, Kwong P D, Graham B S. Structure of RSV fusion glycoprotein trimer bound to a prefusion-specific neutralizing antibody. Science. 2013; 340(6136):1113-7. doi: 10.1126/science.1234914. PubMed PMID: 23618766; PMCID: 4459498.
32. Lee J E, Fusco M L, Hessell A J, Oswald W B, Burton D R, Saphire E O. Structure of the Ebola virus glycoprotein bound to an antibody from a human survivor. Nature. 2008; 454(7201): 177-82. Epub 2008/07/11. doi: nature07082 [pii] 10.1038/nature07082. PubMed PMID: 18615077.
33. Ekiert D C, Bhabha G, Elsliger M A, Friesen R H, Jongeneelen M, Throsby M, Goudsmit J, Wilson I A. Antibody recognition of a highly conserved influenza virus epitope. Science. 2009; 324(5924):246-51. doi: 10.1126/science.1171491. PubMed PMID: 19251591; PMCID: 2758658.
34. Wei C J, Crank M C, Shiver J, Graham B S, Mascola J R, Nabel G J. Next-generation influenza vaccines: opportunities and challenges. Nat Rev Drug Discov. 2020. Epub 2020/02/16. doi: 10.1038/s41573-019-0056-x. PubMed PMID: 32060419.
35. McLellan J S, Pancera M, Carrico C, Gorman J, Julien J P, Khayat R, Louder R, Pejchal R, Sastry M, Dai K, O'Dell S, Patel N, Shahzad-ul-Hussan S, Yang Y, Zhang B, Zhou T, Zhu J, Boyington J C, Chuang G Y, Diwanji D, Georgiev I, Kwon Y D, Lee D, Louder M K, Moquin S, Schmidt S D, Yang Z Y, Bonsignori M, Crump J A, Kapiga S H, Sam N E, Haynes B F, Burton D R, Koff W C, Walker L M, Phogat S, Wyatt R, Orwenyo J, Wang L X, Arthos J, Bewley C A, Mascola J R, Nabel G J, Schief W R, Ward A B, Wilson I A, Kwong P D. Structure of HIV-1 gp120 V1/V2 domain with broadly neutralizing antibody PG9. Nature. 2011; 480(7377):336-43. Epub 2011/11/25. doi: 10.1038/nature10696. PubMed PMID: 22113616; PMCID: 3406929.
36. Pancera M, Majeed S, Ban Y E, Chen L, Huang C C, Kong L, Kwon Y D, Stuckey J, Zhou T, Robinson J E, Schief W R, Sodroski J, Wyatt R, Kwong P D. Structure of HIV-1 gp120 with gp41-interactive region reveals layered envelope architecture and basis of conformational mobility. Proc Natl Acad Sci USA. 2010; 107(3):1166-71. Epub 2010/01/19. doi: 0911004107 [pii]10.1073/pnas.0911004107. PubMed PMID: 20080564; PMCID: 2824281.

1. An antibody or an antigen binding fragment thereof comprising the following combination of complementarity determining regions (CDRs):
a CDR-L1 comprising the sequence RASQGISSWLA (SEQ ID NO:212); a CDR-L2 comprising the sequence AASSLQS (SEQ ID NO:207); a CDR-L3 comprising the sequence QQGNSFPYT (SEQ ID NO:213); a CDR-H1 comprising the sequence GYTFTRY (SEQ ID NO:209); a CDR-H2 comprising the sequence YPGDSD (SEQ ID NO:210); and a CDR-H3 comprising the sequence LPQYCSNGVCQRWFDP (SEQ ID NO:211).

2-3. (canceled)

4. The antibody or an antigen binding fragment thereof according to claim 1, which comprises the following combination of heavy chain and light chain variable regions:
a heavy chain variable region comprising an amino acid sequence having at least 70% identity with the sequence of SEQ ID NO: 35; and a light chain variable region comprising an amino acid sequence having at least 70% identity with the sequence of SEQ ID NO: 36.

5. The antibody or antigen binding fragment thereof according to claim 4, which comprises the following combination of heavy chain and light chain variable regions: a heavy chain variable region comprising the amino acid sequence of SEQ ID NO: 35; and a light chain variable region comprising the amino acid sequence of SEQ ID NO: 36.

6. (canceled)

7. The antibody or antigen binding fragment thereof according to claim 1, which is a fully human antibody.

8. The antibody or antigen binding fragment thereof according to claim 1, which is an IgG antibody.

9. The antibody or antigen binding fragment thereof according to claim 1, which is a recombinant antibody or antigen binding fragment thereof.

10. A conjugate comprising the antibody or antigen binding fragment thereof of claim 1.

11. A nucleic acid comprising a sequence encoding the light and heavy chain of the antibody or antigen binding fragment thereof of claim 1; or a first nucleic acid comprising a sequence encoding the light chain of the antibody or antigen binding fragment thereof of claim 1 and a second nucleic acid comprising a sequence encoding the heavy chain of the antibody or antigen binding fragment thereof of claim 1.

12. (canceled)

13. A mixture comprising the antibody or antigen binding fragment thereof of claim 1, a second antibody or antigen binding fragment thereof comprising one of the following combinations of complementarity determining regions (CDRs):
(a) a light chain CDR1 (CDR-L1) comprising an amino acid sequence having at least 70% identity with the sequence RASQSVSSSYLA (SEQ ID NO:80); a light chain CDR2 (CDR-L2) comprising the sequence GASSRAT (SEQ ID NO:81); a light chain CDR3 (CDR-L3) comprising the sequence QQYGTSPWT (SEQ ID NO:82); a heavy chain CDR1 (CDR-H1) comprising the sequence GFTFTSS (SEQ ID NO:77); a heavy chain CDR2 (CDR-H2) comprising the sequence VVGSGN (SEQ ID NO:78); and a heavy chain CDR3 (CDR-H3) comprising the sequence PSCSGGRCYDGFDI (SEQ ID NO:79);
(b) a CDR-L1 comprising the sequence RASQTISSWLA (SEQ ID NO:86); a CDR-L2 comprising the sequence KASTLES (SEQ ID NO:87); a CDR-L3 comprising the sequence QQYNSYPWT (SEQ ID NO:88); a CDR-H1 comprising the sequence GFTFSDY (SEQ ID NO:83); a CDR-H2 comprising the sequence GSSGSS (SEQ ID NO:84); and a CDR-H3 comprising the sequence DGSYGDYVRGY (SEQ ID NO:85);
(c) a CDR-L1 comprising the sequence TGTSSDVGSYNVVS (SEQ ID NO:92); a CDR-L2 comprising the sequence EVSKRPS (SEQ ID NO:93); a CDR-L3 comprising the sequence CSYAGSSTSWVV (SEQ ID NO:94); a CDR-H1 comprising the sequence GFTFSSY (SEQ ID NO:89); a CDR-H2 comprising the sequence SGSGGS (SEQ ID NO:90); and a CDR-H3 comprising the sequence DDSTSAYYYYYYMDV (SEQ ID NO:91);
(d) a CDR-L1 comprising the sequence KSSQSVLYSSNNKNYLA (SEQ ID NO:98); a CDR-L2 comprising the sequence WASTRES (SEQ ID NO:99); a CDR-L3 comprising the sequence QQYYNSYT (SEQ ID NO:100); a CDR-H1 comprising the sequence GGSISSSSY (SEQ ID NO:95); a CDR-H2 comprising the sequence YYSGS (SEQ ID NO:96); and a CDR-H3 comprising the sequence HPTFSGYEYYFDH (SEQ ID NO:97);
(e) a CDR-L1 comprising the sequence RASQSVNNYLA (SEQ ID NO:214); a CDR-L2 comprising the sequence DASHRAT (SEQ ID NO:215); a CDR-L3 comprising the sequence QQRSNWPLT (SEQ ID NO:216); a CDR-H1 comprising the sequence GFTFSSY (SEQ ID NO:89); a CDR-H2 comprising the sequence NNHGGS (SEQ ID NO:101); and a CDR-H3 comprising the sequence SDTAMVPYNWFDP (SEQ ID NO:102);
(f) a CDR-L1 comprising the sequence RASQSVRSNLA (SEQ ID NO:217); a CDR-L2 comprising the sequence GASTRAT (SEQ ID NO:143); a CDR-L3 comprising the sequence QQYNYWPPYT (SEQ ID NO:218); a CDR-H1 comprising the sequence GGSLNNY (SEQ ID NO:219); a CDR-H2 comprising the sequence NHSGS (SEQ ID NO:220); and a CDR-H3 comprising the sequence GLFLVYYGSGLGGFDY (SEQ ID NO:221);
(g) a CDR-L1 comprising the sequence RASQDISSALA (SEQ ID NO:106); a CDR-L2 comprising the sequence DASSLES (SEQ ID NO:107); a CDR-L3 comprising the sequence QQFNNYPLT (SEQ ID NO:108); a CDR-H1 comprising the sequence GFTFSRY (SEQ ID NO:103); a CDR-H2 comprising the sequence SYDGSN (SEQ ID NO:104); and a CDR-H3 comprising the sequence DLEYYTSGSYSLFDY (SEQ ID NO:105);
(h) a CDR-L1 comprising the sequence RASQSVSSTYLA (SEQ ID NO:111); a CDR-L2 comprising the sequence GASNRAT (SEQ ID NO:112); a CDR-L3 comprising the sequence QQYGSSPPLT (SEQ ID NO:113); a CDR-H1 comprising the sequence GFTFSIY (SEQ ID NO:109); a CDR-H2 comprising the sequence SYDGSN (SEQ ID NO:104); and a CDR-H3 comprising the sequence GPTYSYMDV (SEQ ID NO:110);
(i) a CDR-L1 comprising the sequence QASQDISNYLN (SEQ ID NO:117); a CDR-L2 comprising the sequence DASNLET (SEQ ID NO:118); a CDR-L3 comprising the sequence QQYNNLPLT (SEQ ID NO:119); a CDR-H1 comprising the sequence GFTFSYY (SEQ ID NO:114); a CDR-H2 comprising the sequence YGSGSN (SEQ ID NO:115); and a CDR-H3 comprising the sequence DQRNAYDSFDF (SEQ ID NO:116);
(j) a CDR-L1 comprising the sequence SGSSSNIGNNYVS (SEQ ID NO:123); a CDR-L2 comprising the sequence DNNKRPS (SEQ ID NO:124); a CDR-L3 comprising the sequence GTWDSSLSVVL (SEQ ID NO:125); a CDR-H1 comprising the sequence GFTFGDY (SEQ ID NO:120); a CDR-H2 comprising the sequence RSKAYGGT (SEQ ID NO:121); and a CDR-H3 comprising the sequence DLDYYDSSGYYPTYIDY (SEQ ID NO:122);
(k) a CDR-L1 comprising the sequence TGSGSNIGAGYDVH (SEQ ID NO:222); a CDR-L2 comprising the sequence GNNNRPS (SEQ ID NO:223); a CDR-L3 comprising the sequence QSYDSSLSGPVV (SEQ ID NO:224); a CDR-H1 comprising the sequence GGSISSGNY (SEQ ID NO:126); a CDR-H2 comprising the sequence YTSGS (SEQ ID NO: 127); and a CDR-H3 comprising the sequence DAYYDFLSGYIPTYNWFDP (SEQ ID NO:225);
(l) a CDR-L1 comprising the sequence QASQDISNYLN (SEQ ID NO:117); a CDR-L2 comprising the sequence VASNLET (SEQ ID NO:226); a CDR-L3 comprising the sequence QQFDNLPYT (SEQ ID NO:227); a CDR-H1 comprising the sequence GGSISSGTY (SEQ ID NO:228); a CDR-H2 comprising the sequence YTSGS (SEQ ID NO:127); and a CDR-H3 comprising the sequence EYSSSYYYFYYMDV (SEQ ID NO:128);
(m) a CDR-L1 comprising the sequence QASQDISKYLN (SEQ ID NO:132); a CDR-L2 comprising the sequence DASNLET (SEQ ID NO:118); a CDR-L3 comprising the sequence QQYDNLPTT (SEQ ID NO:133); a CDR-H1 comprising the sequence GFTFSNY (SEQ ID NO:129); a CDR-H2 comprising the sequence LYDGSN (SEQ ID NO:130); and a CDR-H3 comprising the sequence GGGPYCGGGSCWAHYFDY (SEQ ID NO:131);
(n) a CDR-L1 comprising the sequence RASQSVSSIYLA (SEQ ID NO:136); a CDR-L2 comprising the sequence STSSRAV (SEQ ID NO:137); a CDR-L3 comprising the sequence HQYGSSPWT (SEQ ID NO:138); a CDR-H1 comprising the sequence GDSISNY (SEQ ID NO:134); a CDR-H2 comprising the sequence YYSGS (SEQ ID NO:96); and a CDR-H3 comprising the sequence DFSL (SEQ ID NO:135);
(o) a CDR-L1 comprising the sequence RASQSVSSNLA (SEQ ID NO:142); a CDR-L2 comprising the sequence GASTRAT (SEQ ID NO:143); a CDR-L3 comprising the sequence QQYYNWPPWT (SEQ ID NO:144); a CDR-H1 comprising the sequence GFIFSRY (SEQ ID NO:139); a CDR-H2 comprising the sequence SSSTSF (SEQ ID NO:140); and a CDR-H3 comprising the sequence WIGGDSSGYYPDAFDI (SEQ ID NO:141);
(p) a CDR-L1 comprising the sequence RASQSVSSNYLA (SEQ ID NO:229); a CDR-L2 comprising the sequence GASSRAT (SEQ ID NO:81); a CDR-L3 comprising the sequence QQYGSSLYT (SEQ ID NO:230); a CDR-H1 comprising the sequence GDSISSY (SEQ ID NO:145); a CDR-H2 comprising the sequence YYTGS (SEQ ID NO:146); and a CDR-H3 comprising the sequence LGYNSGWYGGYFEY (SEQ ID NO:147);
(q) a CDR-L1 comprising the sequence RASQSVSSNLA (SEQ ID NO:142); a CDR-L2 comprising the sequence GASTRAT (SEQ ID NO:143); a CDR-L3 comprising the sequence QQYNKWPPIT (SEQ ID NO:150); a CDR-H1 comprising the sequence GFTFSSY (SEQ ID NO:89); a CDR-H2 comprising the sequence SYDGIN (SEQ ID NO:148); and a CDR-H3 comprising the sequence MYSGSYLGYFDY (SEQ ID NO:149);
(r) a CDR-L1 comprising the sequence TGSSSNIGAGYDVH (SEQ ID NO:154); a CDR-L2 comprising the sequence DNNNRPS (SEQ ID NO:155); a CDR-L3 comprising the sequence QSYDSSLSGSHVV (SEQ ID NO:156); a CDR-H1 comprising the sequence GGTFSNY (SEQ ID NO:151); a CDR-H2 comprising the sequence IPIFGI (SEQ ID NO:152); and a CDR-H3 comprising the sequence GWWFGELETYYFDY (SEQ ID NO:153);
(s) a CDR-L1 comprising the sequence PGDKLGDKFAC (SEQ ID NO:160); a CDR-L2 comprising the sequence QDNKRPS (SEQ ID NO:161); a CDR-L3 comprising the sequence QAWHSSTVV (SEQ ID NO:162); a CDR-H1 comprising the sequence GYSLNSGY (SEQ ID NO:157); a CDR-H2 comprising the sequence YHSGS (SEQ ID NO:158); and a CDR-H3 comprising the sequence KLVPTAPFDY (SEQ ID NO:159);
(t) a CDR-L1 comprising the sequence SGTSSDVGRYNYVS (SEQ ID NO:165); a CDR-L2 comprising the sequence DVSDRPS (SEQ ID NO:166); a CDR-L3 comprising the sequence TSHTSSTISYVV (SEQ ID NO:167); a CDR-H1 comprising the sequence GFTFSTY (SEQ ID NO:163); a CDR-H2 comprising the sequence SYDGSN (SEQ ID NO:104); and a CDR-H3 comprising the sequence DPHIVVVPAAMRFEA (SEQ ID NO: 164);
(u) a CDR-L1 comprising the sequence RSSQSLLHSNGYNYLD (SEQ ID NO:170); a CDR-L2 comprising the sequence LGSNRAS (SEQ ID NO:171); a CDR-L3 comprising the sequence MQALQTPFT (SEQ ID NO:171); a CDR-H1 comprising the sequence GGSISSY (SEQ ID NO:168); a CDR-H2 comprising the sequence YYSGS (SEQ ID NO:96); and a CDR-H3 comprising the sequence APGATYSSGWYYYYYYMDV (SEQ ID NO:169);
(v) a CDR-L1 comprising the sequence RSSQSLLHINGYNYLD (SEQ ID NO:176); a CDR-L2 comprising the sequence LGSNRAS (SEQ ID NO:171); a CDR-L3 comprising the sequence MQALQTPWT (SEQ ID NO:177); a CDR-H1 comprising the sequence GFPFRNY (SEQ ID NO:173); a CDR-H2 comprising the sequence SSRGDT (SEQ ID NO:174); and a CDR-H3 comprising the sequence VQSGFSYGYGFDY (SEQ ID NO:175);
(w) a CDR-L1 comprising the sequence TGTSSDVGSYNLVS (SEQ ID NO:179); a CDR-L2 comprising the sequence EVSKRPS (SEQ ID NO:93); a CDR-L3 comprising the sequence CSYAGSSTSYVV (SEQ ID NO:180); a CDR-H1 comprising the sequence GFTFSSY (SEQ ID NO:89); a CDR-H2 comprising the sequence SGSGGS (SEQ ID NO:90); and a CDR-H3 comprising the sequence DGAVATGPGYFYFYMDV (SEQ ID NO:178);
(x) a CDR-L1 comprising the sequence RASQGISSALA (SEQ ID NO:182); a CDR-L2 comprising the sequence DASSLES (SEQ ID NO:107); a CDR-L3 comprising the sequence QQFNNYPLT (SEQ ID NO:108); a CDR-H1 comprising the sequence GFTFSSY (SEQ ID NO:89); a CDR-H2 comprising the sequence SYDGSN (SEQ ID NO:104); and a CDR-H3 comprising the sequence DLEYYGSGSYSLFDY (SEQ ID NO:181);
(y) a CDR-L1 comprising the sequence KSSQSVLYSSNNKNYLA (SEQ ID NO:98); a CDR-L2 comprising the sequence WASTRES (SEQ ID NO:99); a CDR-L3 comprising the sequence QQYYSTPLT (SEQ ID NO:231); a CDR-H1 comprising the sequence GFTFSSY (SEQ ID NO:89); a CDR-H2 comprising the sequence SYDGSN (SEQ ID NO:104); and a CDR-H3 comprising the sequence GGGGYNTFFDY (SEQ ID NO:183);
(z) a CDR-L1 comprising the sequence QASQDISNFLN (SEQ ID NO:186); a CDR-L2 comprising the sequence DASNLET (SEQ ID NO:118); a CDR-L3 comprising the sequence QHYTT (SEQ ID NO:187); a CDR-H1 comprising the sequence GGSIRSYSH (SEQ ID NO:184); a CDR-H2 comprising the sequence YYSGS (SEQ ID NO:96); and a CDR-H3 comprising the sequence TIPTYDDILTGYQFDY (SEQ ID NO:185);
(aa) a CDR-L1 comprising the sequence SGDKLGDKYVC (SEQ ID NO:190); a CDR-L2 comprising the sequence QDTKRPS (SEQ ID NO:191); a CDR-L3 comprising the sequence QAWDSSTGV (SEQ ID NO:192); a CDR-H1 comprising the sequence GFTFSSY (SEQ ID NO:89); a CDR-H2 comprising the sequence KQDGSE (SEQ ID NO:188); and a CDR-H3 comprising the sequence VGLSSWYFEY (SEQ ID NO:189);
(bb) a CDR-L1 comprising the sequence TGSSSDVGGYDFVS; a CDR-L2 comprising the sequence DVTNRPS (SEQ ID NO:197); a CDR-L3 comprising the sequence SSYTSSSTRV (SEQ ID NO:198); a CDR-H1 comprising the sequence GYIFTDY (SEQ ID NO:193); a CDR-H2 comprising the sequence NPNSGG (SEQ ID NO: 194); and a CDR-H3 comprising the sequence EASLNRSRYYSSGGTVYYYYYYMDV (SEQ ID NO:195);
(cc) a CDR-L1 comprising the sequence RASQGIRNDLG (SEQ ID NO:232); a CDR-L2 comprising the sequence AASSLQS (SEQ ID NO:207); a CDR-L3 comprising the sequence LQYNSYPRT (SEQ ID NO:233); a CDR-H1 comprising the sequence GYSISSGY (SEQ ID NO:199); a CDR-H2 comprising the sequence YHSGS (SEQ ID NO: 158); and a CDR-H3 comprising the sequence DHGSYDFWSGYSRDAFDI (SEQ ID NO:200);
(dd) a CDR-L1 comprising the sequence RASQSISSWLA (SEQ ID NO:234); a CDR-L2 comprising the sequence KASSLES (SEQ ID NO:235); a CDR-L3 comprising the sequence QQYNTYSFT (SEQ ID NO:236); a CDR-H1 comprising the sequence GFSVSSN (SEQ ID NO:201); a CDR-H2 comprising the sequence YSGGS (SEQ ID NO:202); and a CDR-H3 comprising the sequence GYGDSQR (SEQ ID NO:203); or
(ee) a CDR-L1 comprising the sequence RASQSINNYLN (SEQ ID NO:206); a CDR-L2 comprising the sequence AASSLQS (SEQ ID NO:207); a CDR-L3 comprising the sequence QQSYSPYT (SEQ ID NO:208); a CDR-H1 comprising the sequence GGSVSSDNY (SEQ ID NO:204); a CDR-H2 comprising the sequence YYSGS (SEQ ID NO:96); and a CDR-H3 comprising the sequence GFVATYYYYMDV (SEQ ID NO:205).

14. (canceled)

15. The mixture of claim 13, wherein the second antibody or antigen binding fragment thereof comprises the following combination of heavy chain and light chain variable regions: a heavy chain variable region comprising an amino acid sequence having at least 70% identity with the sequence of SEQ ID NO: 1; and a light chain variable region comprising an amino acid sequence having at least 70% identity with the sequence of SEQ ID NO: 2.

16. (canceled)

17. A pharmaceutical composition comprising the antibody or antigen binding fragment thereof of claim 1, and a pharmaceutically acceptable excipient.

18. The pharmaceutical composition of claim 17, wherein the pharmaceutical composition is in the form of an aerosol or an injectable solution.

19-20. (canceled)

21. A method for preventing or treating a betacoronavirus infection or a related disease, or for reducing the risk of developing a betacoronavirus-related disease or the severity of a betacoronavirus-related disease, in a subject in need thereof, the method comprising administering to the subject an effective amount of the pharmaceutical composition of claim 17.

22-23. (canceled)

24. The method of claim 21, wherein the betacoronavirus is a sarbecovirus.

25. The method of claim 24, wherein the sarbecovirus is SARS-CoV-2.

26. The method of claim 25, wherein the SARS-CoV-2 is a variant of the Wuhan original SARS-CoV-2 strain.

27. The method of claim 21, wherein the antibody, antigen-binding fragment thereof, nucleic acid, mixture, or pharmaceutical composition is administered with (i) at least one additional anti-SARS-CoV-2 antibody or antigen-binding fragment thereof, or nucleic acid(s) encoding said at least one additional anti-SARS-CoV-2 antibody or antigen-binding fragment thereof; and/or (ii) at least one antiviral or anti-inflammatory drug.

28. The method of claim 21, wherein the subject is an immunosuppressed or immunocompromised subject.

29-47. (canceled)
